(19) United States
(12) Patent Application Publication
(10) Pub. No.: US 2024/0294616 A1
(43) Pub. Date: Sep. 5, 2024

(54) CAMELIDAE SINGLE-DOMAIN ANTIBODIES AGAINST YERSINIA PESTIS AND METHODS OF USE

(71) Applicant: Government of the United States as represented by the Secretary of the Air Force, Wright-Patterson AFB, OH (US)

(72) Inventors: Camilla A. Mauzy, Enon, OH (US); Serge Victor Marie Muyldermans, Brussels (BE)

(73) Assignee: Government of the United States as represented by the Secretary of the Air Force, Wright-Patterson AFB, OH (US)

(21) Appl. No.: 18/663,247

(22) Filed: May 14, 2024

Related U.S. Application Data
(63) Continuation of application No. 17/141,554, filed on Jan. 5, 2021, now Pat. No. 12,030,932.
Application No. 17/141,554 is a continuation of application No. 16/023,723, filed on Jun. 29, 2018, now Pat. No. 11,339,208.
Application No. 16/023,723 is a continuation of application No. 13/906,386, filed on May 31, 2013, now abandoned.
(60) Provisional application No. 61/653,488, filed on May 31, 2012.

(51) Int. Cl. C07K 16/12

(52) U.S. Cl. CPC C07K 16/1228

(57) ABSTRACT

Single-domain antibodies (SAbs) against three Yersinia pestis surface proteins (LcrV, YscF, and F1), nucleic acid sequences encoding the SAbs, and polypeptides comprising two or more SAbs capable of recognizing two or more epitopes and/or antigens. The present invention further includes methods for preventing or treating Y. pestis infections in a patient; methods for detecting and/or diagnosing Y. pestis infections; and devices and methods for identifying and/or detecting Y. pestis on a surface and/or in an environment.

### CROSS-REFERENCE TO RELATED APPLICATIONS

This application is a continuation of U.S. application Ser. No. 17/141,554, filed Jan. 5, 2021, which was a continuation of U.S. application Ser. No. 16/023,723, filed Jun. 29, 2018, now U.S. Pat. No. 11,339,208 issued May 24, 2022, which was a continuation of U.S. application Ser. No. 13/906,386, filed May 31, 2013 (abandoned), which claimed the benefit of and priority to U.S. Provisional Application No. 61/653,488, filed on May 31, 2012. The disclosure of each application is incorporated herein by reference, in its entirety.

### RIGHTS OF THE GOVERNMENT

The invention described herein may be manufactured and used by or for the Government of the United States for all governmental purposes without the payment of any royalty.

### REFERENCE TO AN ELECTRONIC SEQUENCE LISTING

The contents of the electronic sequence listing (AFD-1216CON8 Sequence Listing.xml; Size: 307 KM; and Date of Creation: May 13, 2024) is herein incorporated by reference in its entirety.

### FIELD OF THE INVENTION

This invention relates generally to the field of single-domain antibodies. More particularly, it relates to single-domain antibodies and polypeptides against Yersinia pestis, nucleic acid sequences encoding the single-domain antibodies, and methods of using the same.

### BACKGROUND OF THE INVENTION

Increasing threats of bioterrorism have led to the development of new diagnostic and therapeutic tools for pathogens that can potentially be used as biological weapons. Many of these pathogens, such as the causative agents of plague, anthrax, and tularemia, are relatively easy to manipulate via genetic engineering and may be designed to evade detection by sensor devices. Many of these biological weapons candidates also display resistance to current medical treatments. To be useful, a diagnostic tool must be sensitive and specific, as well as able to withstand the extreme conditions often encountered in the field. The value of a therapeutic tool is largely determined by parameters such as toxicity, immunogenicity, and efficacy after administration. In addition, the therapeutic tool may be required to treat large number of people in the event of a bioterrorism attack. All of these requirements highlight the importance of a long shelf life and the production costs of biological weapon-related diagnostics and therapeutics.

Members of the family Camelidae, which includes alpacas, camels, and llamas, produce conventional antibodies, as well as antibodies consisting only of a dimer of heavy-chain polypeptides. The N-terminal domain of these heavy chain-only antibodies, which is referred to as VHH, is variable in sequence, and it is the sole domain that interacts with the cognate antigen. Because of their small size (12-15 kDa, 2.2 nm diameter, and 4 nm height), VHHs are also known as single-domain antibodies (SAbs), which are commercially-available as NANOBODIES (NANOBODY and NANOBODIES are registered trademarks of Ablynx N.V., Belgium).

SAbs make attractive as tools for biological weapon detection due to their high affinity and specificity for their respective targets and their high stability and solubility. Their small size gives SAbs the unique ability to recognize and bind to areas of an antigen that are often not normally accessible to full-size antibodies due to steric hindrance and other size constraints. In addition, SAbs may be economically produced in large quantities, and their sequences are relatively easy to tailor to a specific application. These properties, as well as their low immunogenicity, make SAbs uniquely suited for detection, diagnostics, and immunotherapeutics.

### SUMMARY OF THE INVENTION

The present invention includes a composition comprising at least one single-domain antibody against one or more Yersinia pestis (Y. pestis) surface proteins, in which the one or more Y. pestis surface proteins are selected from the group consisting of YscF, F1, and LcrV, with each single-domain antibody comprising four framing regions (FRs) and three complementarity determining regions (CDRs), in which the at least one single-domain antibody is selected from the group consisting of: (1) at least one single-domain antibody comprising one CDR1 sequence selected from the group consisting of SEQ ID NOs:1-7, one CDR2 sequence selected from the group consisting of SEQ ID NOs:27-33, and one CDR3 sequence selected from the group consisting of SEQ ID NOs:54-60; (2) at least one single-domain antibody comprising one CDR1 sequence selected from the group consisting of SEQ ID NOs:8-19, one CDR2 sequence selected from the group consisting of SEQ ID NOs:34-47, and one CDR3 sequence selected from the group consisting of SEQ ID NOs:61-71, AEY, and PGY; and (3) at least one single-domain antibody comprising one CDR1 sequence selected from the group consisting of SEQ ID NOs:20-26, one CDR2 sequence selected from the group consisting of SEQ ID NOs:48-53, and one CDR3 sequence selected from the group consisting of SEQ ID NOs:72-78 and GNI, with the four framing regions of each single-domain antibody comprising one FR1 sequence selected from the group consisting of SEQ ID NOs:79-102, one FR2 sequence selected from the group consisting of SEQ ID NOs:103-120, one FR3 sequence selected from the group consisting of SEQ ID NOs:121-146, and one FR4 sequence selected from the group consisting of SEQ ID NOs:147-153.

In one embodiment, the at least one single-domain antibody is selected from the group consisting of SEQ ID NOs:154-160, 168-185, and 204-217. In a further embodiment, the at least one single-domain antibody further comprises at least one of a protein tag, a protein domain tag, or a chemical tag.

In one embodiment, the composition comprises a plurality of single-domain antibodies against a single Y. pestis surface protein. In another embodiment, at least a portion of the plurality of single-domain antibodies is against different epitopes on the single Y. pestis surface protein. In another embodiment, the composition comprises a plurality of single-domain antibodies against at least two Y. pestis surface proteins.

In an alternative embodiment, the composition comprises a plurality of single-domain antibodies further comprising a polypeptide. In one embodiment, the plurality of single-domain antibodies comprising the polypeptide are against a single Y. pestis surface protein. In another embodiment, at least a portion of the plurality of single-domain antibodies comprising the polypeptide are against different epitopes on the single Y. pestis surface protein. In another embodiment, the plurality of single-domain antibodies comprising the polypeptide are against at least two Y. pestis surface proteins.

In a further embodiment, the polypeptide comprises a fusion protein. In another embodiment, the polypeptide comprises a multivalent protein complex, with the single-domain antibodies being joined together with at least one linker molecule. In a further embodiment, at least one of the plurality of single-domain antibodies comprising the polypeptide further comprises at least one of a protein tag, a protein domain tag, or a chemical tag.

The present invention further includes at least one isolated nucleotide sequence encoding the at least one single-domain antibody, wherein the at least one isolated nucleotide sequence is selected from the group consisting of SEQ ID NOs:164-170, 189-206, and 221-234.

### BRIEF DESCRIPTION OF THE DRAWINGS

FIG. 1 is a graph of the binding response of IgG isolated from an immune alpaca to Y. pestis YscF (ELISA).

FIG. 2 is a graph of the binding response of IgG isolated from an immune alpaca to Y. pestis F1 (ELISA).

FIG. 3 is a graph of the binding response of IgG isolated from an immune alpaca to Y. pestis LcrV (ELISA).

FIG. 4 is a graph of polyclonal phage ELISA testing after each round of panning to isolate YscF-specific phages.

FIG. 5 is a graph of the ELISA for the presence of YscF-specific SAbs in the periplasmic extract of positive colonies.

FIG. 6 is a graph of polyclonal phage ELISA testing after each round of panning to isolate F1-specific phages.

FIGS. 7A-B are graphs of the ELISA for the presence of F1-specific SAbs in the periplasmic extract of positive colonies.

FIG. 8 is a graph of polyclonal phage ELISA testing after each round of panning to isolate LcrV-specific phages.

FIGS. 9A-B are graphs graph of the ELISA for the presence of LcrV-specific SAbs in the periplasmic extract of positive colonies.

FIG. 10 is a protein sequence alignment of seven exemplary YscF SAbs according to the present invention.

FIGS. 11A-B are protein sequence alignments of eighteen exemplary F1 SAbs according to the present invention.

FIGS. 12A-B are the protein sequence alignment of fourteen exemplary LcrV SAbs according to the present invention.

FIGS. 13A-B are the double-referenced sensorgrams obtained on the BIACORE T200 sensor instrument for selected LcrV SAbs.

FIGS. 14A-B are the double-referenced sensorgrams obtained on the BIACORE T200 sensor instrument for the two LcrV SAbs demonstrating the best binding capabilities.

FIGS. 15A-C are sequence alignments of nucleic acid sequences encoding the exemplary YscF SAbs according to the present invention.

FIGS. 16A-H are sequence alignments of nucleic acid sequences encoding the exemplary F1 SAbs according to the present invention.

FIGS. 17A-F are sequence alignments of nucleic acid sequences encoding the exemplary LcrV SAbs according to the present invention.

### DETAILED DESCRIPTION OF THE INVENTION

The present invention includes single-domain antibodies (SAbs) against three Yersinia pestis (Y. pestis) surface proteins (LcrV, YscF, and F1), the nucleic acids encoding the SAbs, and polypeptides comprising two or more SAbs capable of recognizing one or more Y. pestis surface proteins or epitopes. The present invention further includes methods for preventing or treating Y. pestis infections in a patient; methods for detecting and/or diagnosing Y. pestis infections; and devices and methods for identifying and/or detecting Y. pestis on a surface and/or in an environment.

Y. pestis, the gram-negative Bacillus that causes plague, is considered a Class A biological weapon. Y. pestis infections occur in three different ways: infection of the lymph nodes (bubonic), the lungs (pneumonic), or the blood (septicemic). The most serious, contagious, and often fatal mode of plague is pneumonic plague, which may be caused by inhalation of contaminated respiratory droplets from another infected person or from intentional release of aerosolized plague pathogen. While Y. pestis infections are treatable with antibiotics, diagnosis and treatment are often delayed. In the case of pneumonic plague, the early symptoms such as fever, headache, and nausea may easily be mistaken for more common illnesses, delaying proper diagnosis and treatment during the early stages of the disease and greatly increasing the chances of death. Untreated pneumonic plague has a mortality rate of almost 100%. In the case of battlefield personnel and persons stationed or living in rural areas, access to proper health care may be further limited by distance and availability.

Of particular interest for detection and treatment are three Y. pestis surface proteins, LcrV, YscF, and F1. LcrV is a 37 kDa virulence factor that is secreted and expressed on the Y. pestis cell surface prior to bacterial interaction with host cells, making it an excellent antigenic protein for antibody capture. It has been shown that anti-LcrV antibodies can block the delivery of Yops, a set of virulence proteins exported into the host cell upon contact. Additionally, it has been shown that a single sensitive, specific antibody could be used to capture LcrV from Y. pestis, Y. pseudotuberculosis, and Y. enterocolitica. The functional determination of LcrV provides a possible reason for the success of anti-LcrV Ab immunotherapeutics as it is hypothesized that the anti-LcrV/Ab complex prevents the formation and function of the tip complex, thus interfering with the translocation of virulent Yops critical to infection. YscF has also been implicated as one of the “needle” proteins involved in T3SS injection of the virulent Yops proteins across eukaryotic membranes upon cell contact. Recent work using purified YscF to initiate an active immune response indicates that YscF-vaccinated mice have significant protection to a Y. pestis challenge. As with LcrV, these data indicate that YscF is an excellent antigen target for immunotherapeutic uses. F1 protein, which is a Y. pestis capsule protein, has likewise been identified as a potential therapeutic target and is one of the principal immunogens in currently available plague vaccines. Among other roles, F1 is thought to be involved in preventing Y. pestis uptake by macrophages.

SAbs in general, including the presently disclosed Y. pestis SAbs, comprise four framework regions (FRs) interrupted by three complementarity determining regions (CDRs) to yield the following general structure:

FR1-CDR1-FR2-CDR2-FR3-CDR3-FR4.

Like many SAbs, the CDR3 sequence of the presently disclosed Y. pestis SAbs is generally the most crucial in determining antigen specificity. SAbs directed against a particular antigen generally demonstrate some degree of homology or sequence identity between each FR and CDR. Where two nucleotide or amino acid sequences are the same length when aligned, the term “sequence identity” as used herein relates to the number of positions with identical nucleotides or amino acids divided by the total number of nucleotides or amino acids. The number of identical nucleotides or amino acids is determined by comparing corresponding positions of a designated first sequence (usually a reference sequence) with a second sequence. Where two nucleotide or amino acid sequences are of different length when aligned, the term “sequence identity” as used herein relates to the number of positions with identical nucleotides or amino acids divided by the number of nucleotides or amino acids in the designated or reference sequence. Any addition, deletion, insertion, or substitution of a nucleotide or amino acid is considered a difference when calculating the sequence identity. The degree of sequence identity may also be determined using computer algorithms, such algorithms may include, for example, commercially-available Basic Local Alignment Search Tool, also known as BLAST (U.S. National Library of Medicine, Bethesda, MD).

Y. pestis SAbs according to the present invention may be used as components of in vivo and in vitro assays and may also be used diagnostic testing and imaging. The generally low toxicity and immunogenicity of SAbs further makes the present Y. pestis SAbs promising active and passive immunotherapeutic tools, particularly for self-administered fieldable therapeutics. In the case of an outbreak or a biological weapon attack, a self-administered treatment could provide sufficient temporary immunity and sufficiently slow the onset and progress of the disease to allow a person exposed to Y. pestis to reach a hospital for diagnosis and treatment. The SAbs may be introduced by any suitable method including intravenous and subcutaneous injection, oral ingestion, inhalation, and topical administration. The SAbs may bind to extracellular epitopes and antigens and may also bind to intracellular targets after introduction into the host cell by phagocytosis or other mechanisms. In addition, the Y. pestis SAbs may be useful for decontamination and as field-stable capture elements for real-time biological weapon detection and quantitation.

Many of the presently disclosed Y. pestis SAbs demonstrate full functionality and high affinity for their respective antigen targets, which is likely due to the ability of SAbs to bind to protein clefts that are often inaccessible to larger, conventional antibodies. This ability to access areas located in interior pockets may allow therapeutic and detection tools based on the present Y. pestis SAbs to detect multiple strains of the pathogen, as well as related organisms in the Yersinia genus. SAb-based tools and techniques may also be less susceptible to genetic engineering of pathogen surface proteins and epitopes designed to elude current detectors and to circumvent immunity conferred by conventional vaccination.

The Y. pestis SAbs according to the present invention may be quickly, easily, and inexpensively produced in large quantities in a bacterial expression system such as E. coli with little or no loss of protein activity and little or no need for post-translational modification. In addition, the SAbs are stable within a wide range of temperature, humidity, and pH. This stability may allow for stockpiling and long-term storage of the SAbs and SAb-based detection, diagnostic, and therapeutic tools in preparation for Y. pestis outbreaks and/or a bioterrorism attack, all without the need for costly climate control and/or monitoring. The stability of SAbs in extreme environments may further allow for reusable sensors and detection devices.

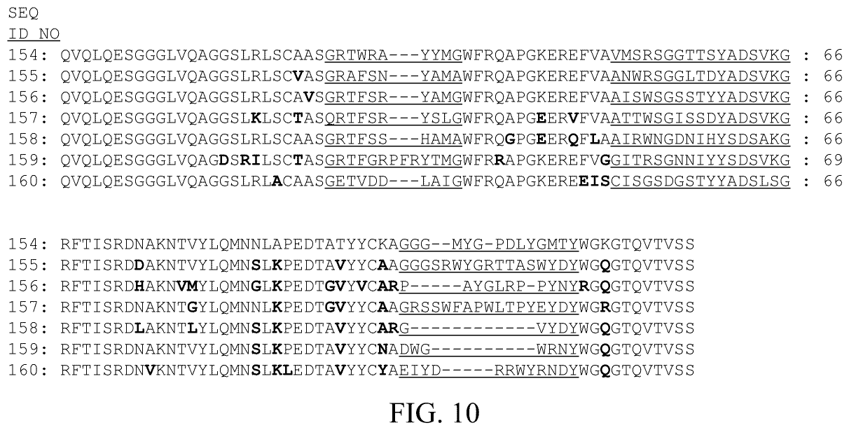

The following examples and methods are presented as illustrative of the present invention or methods of carrying out the invention, and are not restrictive or limiting of the scope of the invention in any manner. Amino acid residues will be according to the standard three-letter or one-letter amino acid code as set out in Table 1. The materials and methods used in Examples 1-4 are described, for example, in Antibody Engineering, Eds. R. Kontermann & S. Dübel, Springer-Verlag, Berlin Heidelberg (2010) Isolation of antigen-specific Nanobodies, Hassanzadeh Ghassabeh Gh., et al., Vol. 2, Chapter 20, pp. 251-266. Exemplary combinations of individual FR and CDR regions are shown in Table 2, and complete SAb protein sequences isolated according to the following Examples are listed in Tables 3, 5, and 7. Unique sequences (individual CDRs and FRs and complete SAb sequences) are each assigned a SEQ ID NO; sequences comprising less than four amino acids are not assigned a SEQ ID NO. As seen in FIGS. 10-12, some SAbs share 100% sequence identity in one or more CDRs and/or FRs because the SAbs are either from clonally-related B-cells or from the same B-cell with diversification due to PCR error during library construction.

### EXAMPLE 1

### Antibody Development and Construction of a VHH Library

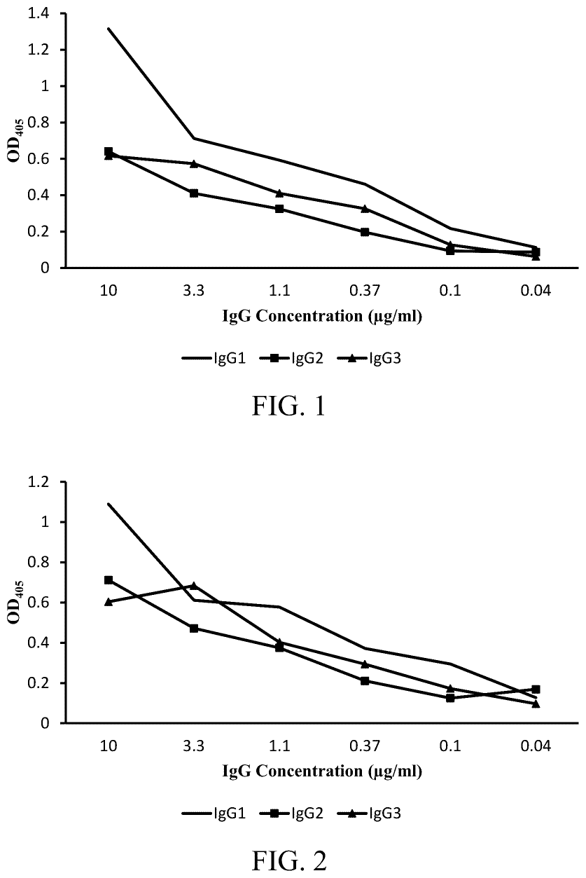

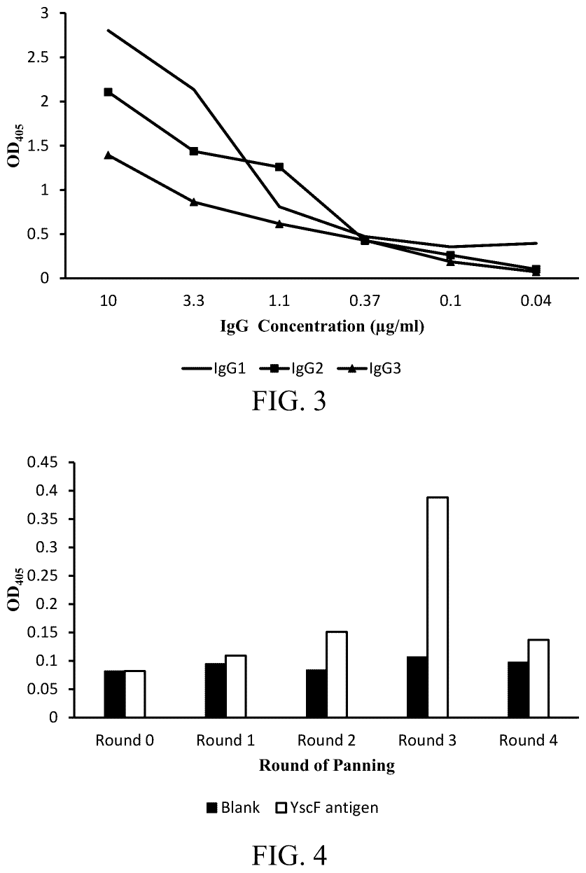

All SAbs were developed using proteins (antigen) expressed from genes isolated from Y. pestis KIM5 (avirulent pgm−), which is similar in sequence to the same protein set in Y. pestis virulent strains (pgm+). An alpaca was injected subcutaneously on days 0, 7, 14, 21, 28 and 35, each time with about 165 μg YscF antigen, about 160 μg F1 antigen, and about 160 μg LcrV antigen. The same animal may be used for all experiments, but multiple animals may also be used. On day 39, anticoagulated blood was collected from the alpaca for the preparation of plasma and peripheral blood lymphocytes. Using plasma from the immune animal, IgG subclasses were obtained by successive affinity chromatography on protein A and protein G columns and were tested by ELISA to assess the immune response to YscF, F1, and LcrV antigens. FIGS. 1-3 are graphs of the immune response to YscF, F1, and LcrV, respectively, in both conventional (IgG1) and heavy chain (IgG2 & IgG3) antibodies. As seen in FIGS. 1-3, the IgG isolated from the immune animal exhibited a strong response toward all three antigens in both types of antibody.

A VHH library was then constructed and screened for the presence of SAbs specific to YscF, F1, and LcrV. Total RNA was extracted from peripheral blood lymphocytes isolated from the immune alpaca and used as a template for first strand cDNA synthesis with oligo(dT) primer. Using this cDNA, the VHH encoding sequences were amplified by PCR and cloned into the phagemid vector pHEN4. pHEN4 vectors containing the amplified VHH sequences were transformed into electrocompetent cells to obtain a VHH library of about 1-2×10⁸ independent transformants. About 75-93% of transformants harbored vectors with the correct insert sizes. Antigen-specific SAbs were then selected from a phage display library.

### EXAMPLE 2

### Isolation of YscF SAbs

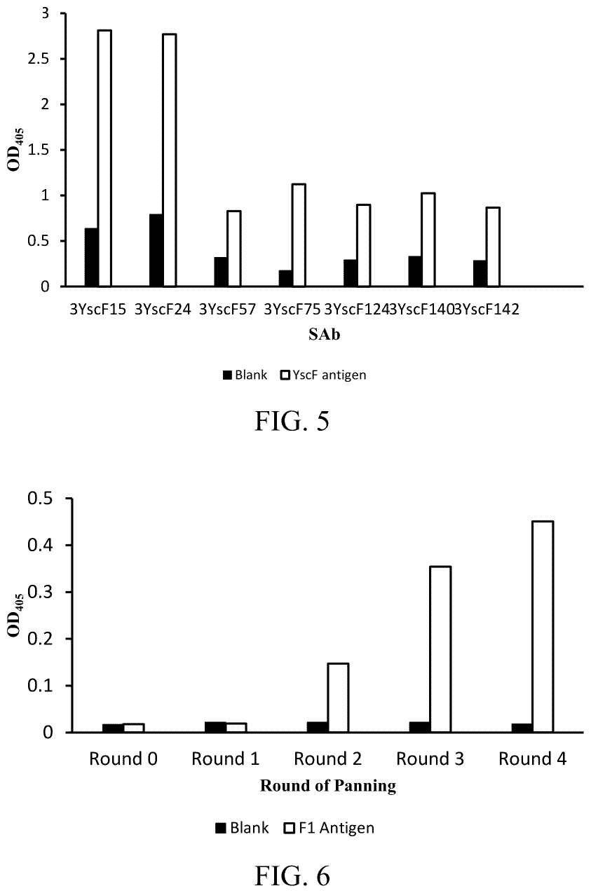

For the YscF antigen, the VHH library was subjected to four consecutive rounds of panning, performed on solid-phase coated antigen (concentration: 700 μg/ml, 30 μg/well, in 25 mM Tris (pH not tested), 150 mM NaCl, 0.05% Tween-20, and 1 mM EDTA). The enrichment for antigen-specific phages after each round of panning was assessed by comparing the number of phages eluted from antigen-coated wells with the number of phages eluted from negative control (only blocked) wells. The enrichment was also evaluated by polyclonal phage ELISA, which is shown in FIG. 4. These experiments suggested that the phage population was enriched for antigen-specific phages only after the third round of panning. In total, 385 individual colonies (95, 143, and 47 from second, third, and fourth rounds, respectively) were randomly selected and analyzed by ELISA for the presence of YscF-specific SAbs in their periplasmic extracts. Out of these 385 colonies, 19 colonies (all from the third round) scored positive. Sequencing of positive colonies identified seven different SAbs, and the ELISA results for these seven SAbs are shown in FIG. 5. The protein sequences of the seven exemplary YscF SAbs according to the present invention are shown in Table 3, and the nucleic acid sequences encoding the seven exemplary YscF SAbs are shown in Table 4.

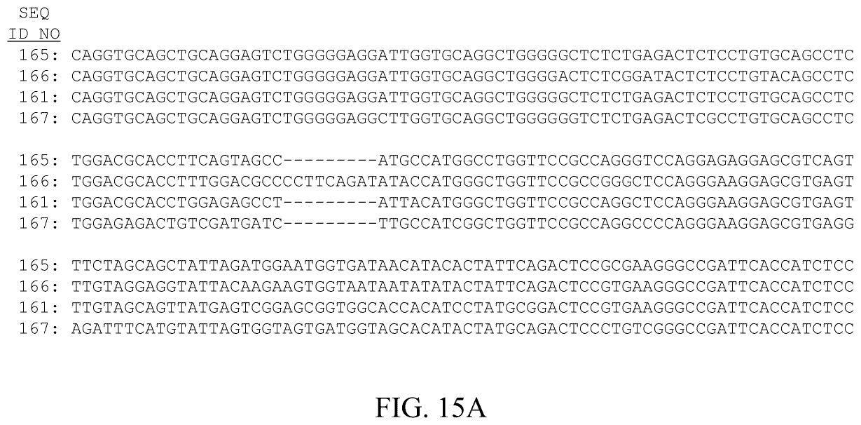

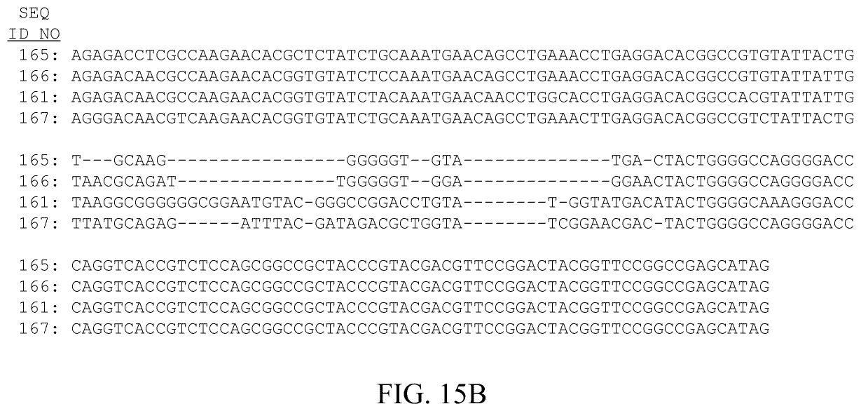

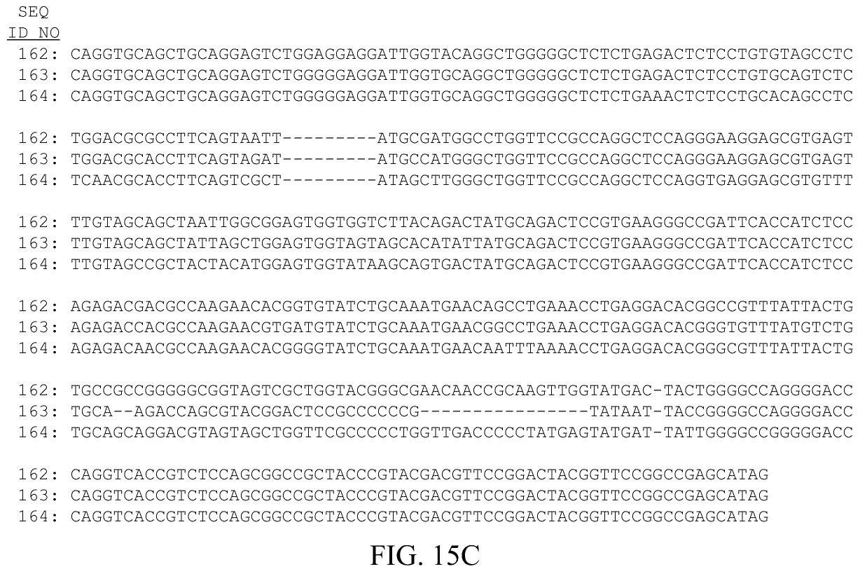

FIG. 10 is a protein sequence alignment of the seven exemplary YscF SAbs listed in Table 3, and FIGS. 15A-C are sequence alignments of the nucleic acid sequences listed in Table 4. Gaps are introduced in the sequences contained in FIGS. 10 and 15A-C as needed in order to align the respective protein and nucleic acid sequences with one another. Referring to FIG. 10, the three CDRs are underlined in each sequence. The CDRs are defined according to the Kabat numbering system [Kabat, E. A., et al., (1991) Sequences of Proteins of Immunological Interest, Fifth Edition, NIH Publication No. 91-3242, US Department of Health and Human Services, Bethesda, MD]. The differences in the four FRs of each SAb (if any), as compared with 3YscF57 (SEQ ID NO:154), are in bold; any differences between the three CDRs of each SAb are not otherwise indicated. The seven exemplary YscF SAb sequences depicted in FIG. 10 and listed in Table 3 represent seven different groups i.e. they originate from seven clonally-unrelated B-cells.

### EXAMPLE 3

### Isolation of F1 SAbs

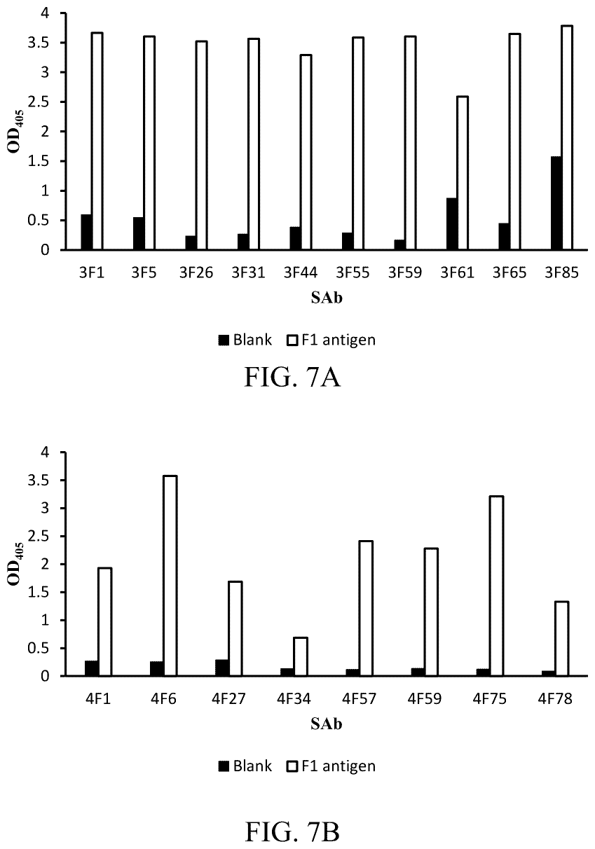

For the F1 antigen, the library was subjected to four consecutive rounds of panning, performed on solid-phase coated antigen (concentration: 200 μg/ml, 20 μg/well, in the presence of 0.005% Tween-20). The enrichment for antigen-specific phages after each round of panning was assessed by comparing the number of phages eluted from antigen-coated wells with the number of phages eluted from negative control (blocked only) wells. The enrichment was also evaluated by polyclonal phage ELISA, which is shown in FIG. 6. These experiments suggested that the phage population was enriched for antigen-specific phages only after the third and fourth rounds of panning. In total, 285 individual colonies from second, third, and fourth rounds of panning (95 from each round) were randomly selected and analyzed by ELISA for the presence of F1-specific SAbs in their periplasmic extracts. Out of these 285 colonies, 55 scored positive (0, 29, and 26 from second, third, and fourth rounds, respectively). Sequencing of these 55 positive colonies identified 18 different SAbs, and the ELISA results for these 19 SAbs are shown in FIGS. 7A-B. The protein sequences of 18 exemplary F1 SAbs according to the present invention are shown in Table 5, and the nucleic acid sequences encoding the 18 F1 SAbs are shown in Table 6.

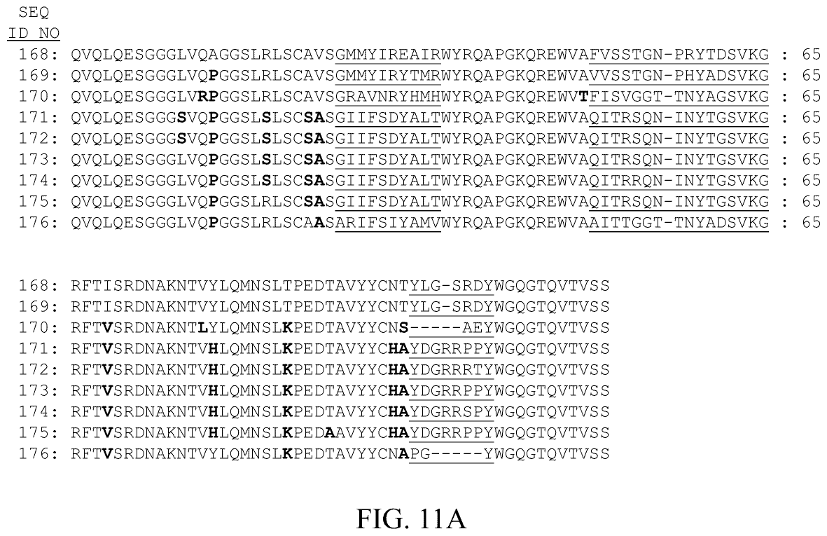

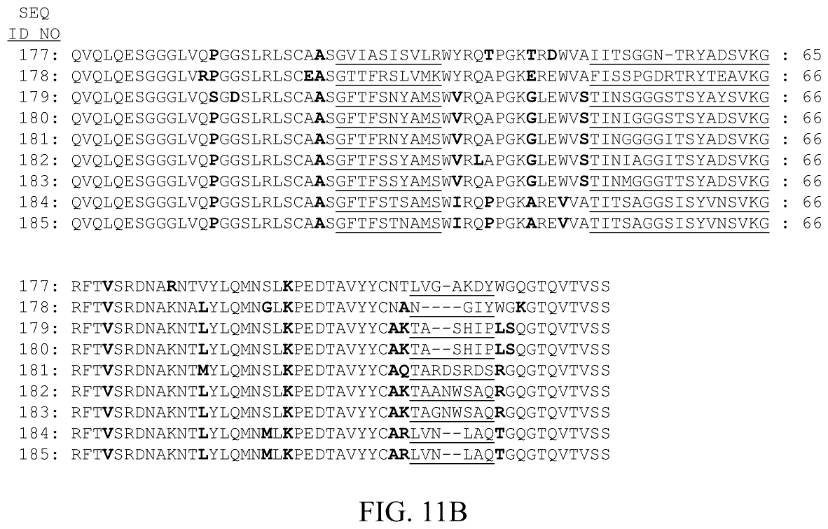

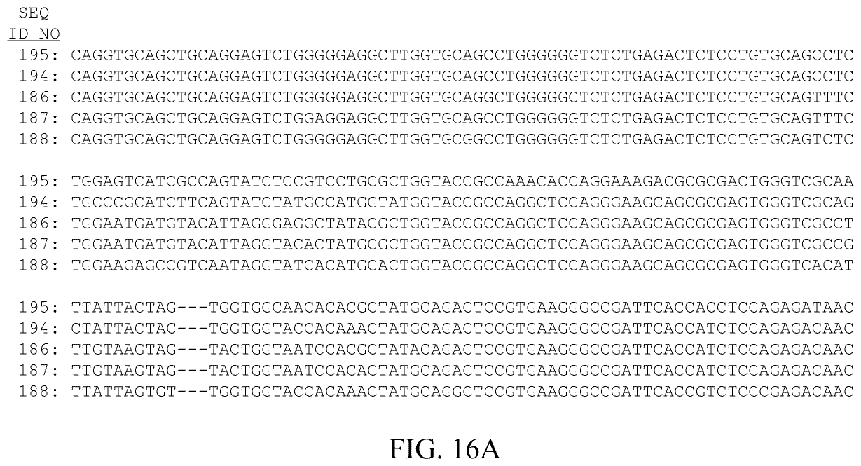

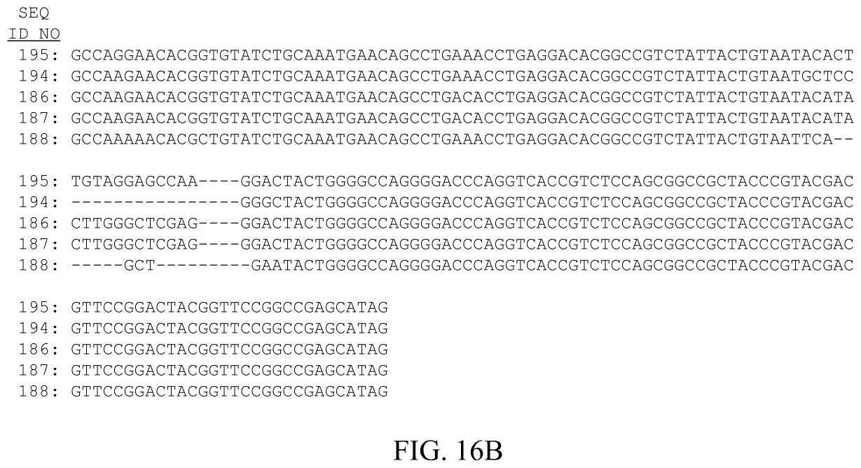

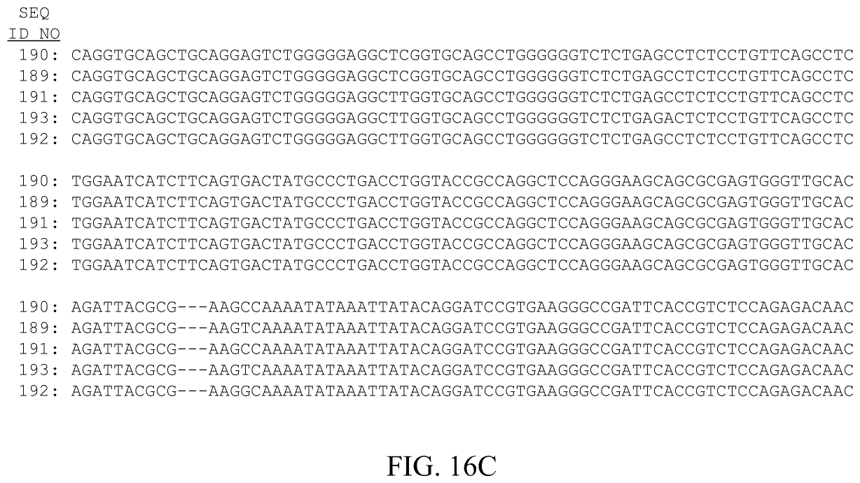

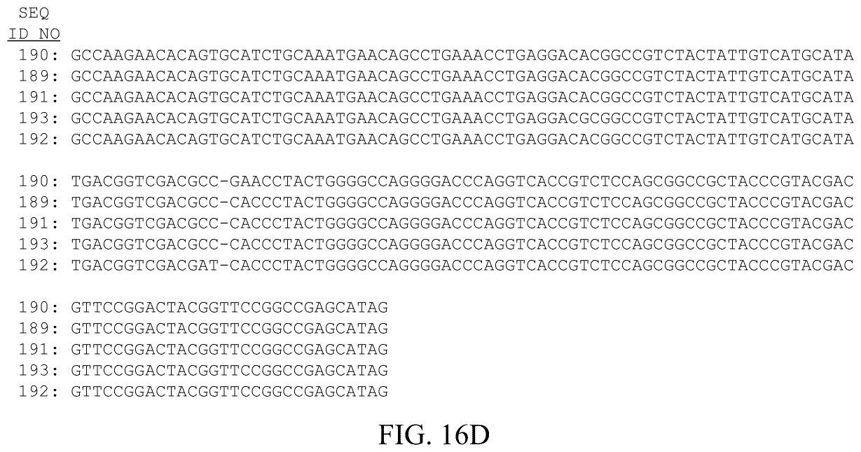

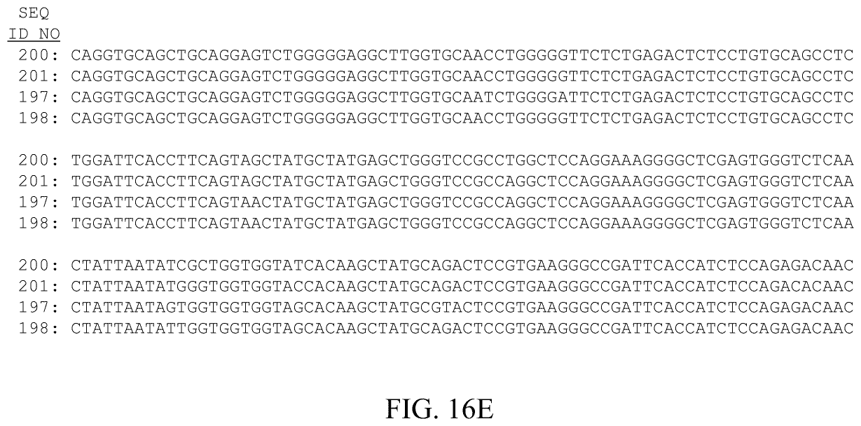

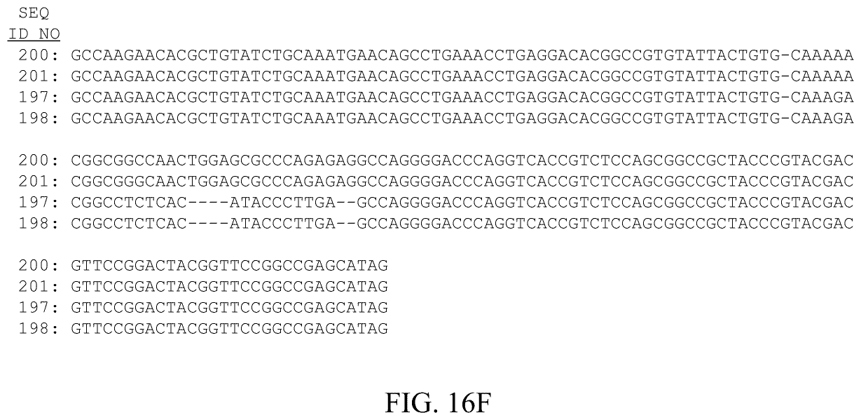

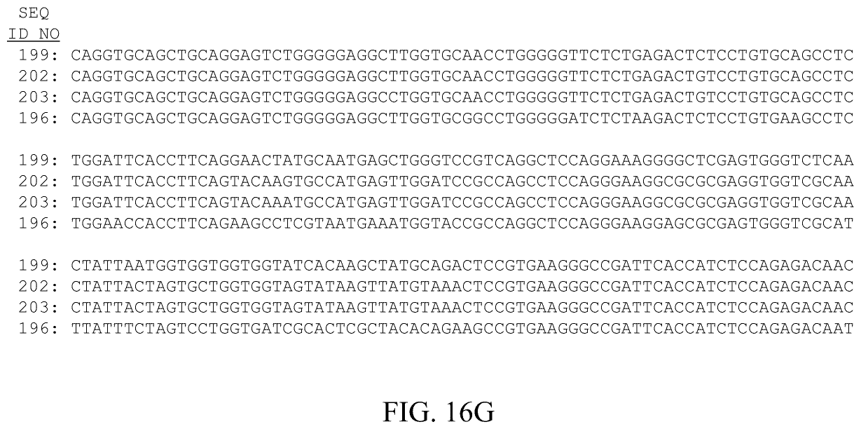

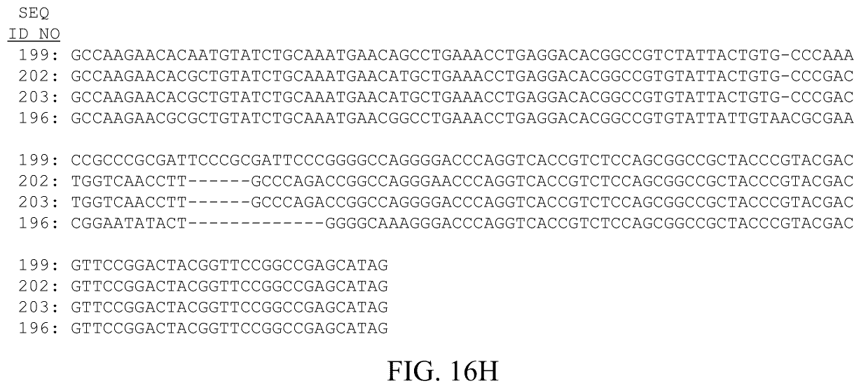

FIGS. 11A-B are protein sequence alignments of the 18 exemplary F1 SAbs listed in Table 5, and FIGS. 16A-H are sequence alignments of the nucleic acid sequences listed in Table 6. Gaps are introduced in the sequences in FIGS. 11A-B and 16A-H as needed in order to align the protein and nucleic acid sequences with one another. Referring to FIGS. 11A-B, the three CDRs are underlined in each sequence. The CDRs are defined according to the Kabat numbering system. The differences in the four FRs of each SAb (if any), as compared with 3F55 (SEQ ID NO:168), are in bold; any differences between the three CDRs of each SAb are not otherwise indicated. The 18 exemplary F1 SAbs shown in FIGS. 11A-B and listed in Table 5 represent 10 different groups, which are listed in Table 9. SAbs belonging to the same group are very similar, especially in the CDR3 region, and their amino acid sequences suggest that they are either from clonally-related B-cells resulting from somatic hypermutation or from the same B-cell with diversification due to PCR error during library construction.

### EXAMPLE 4

### Isolation of LcrV SAbs

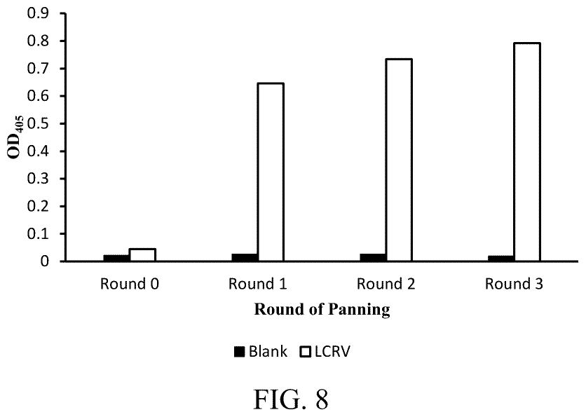

For the LcrV antigen, the library was subjected to three consecutive rounds of panning, performed on solid-phase coated antigen (concentration: 200 μg/ml, 20 μg/well). The enrichment for antigen-specific phages after each round of panning was assessed by comparing the number of phages eluted from antigen-coated wells with the number of phages eluted from negative control (blocked only) wells. The enrichment was also evaluated by polyclonal phage ELISA, which is shown in FIG. 8. These experiments suggested that the phage population was enriched for antigen-specific phages after the first, second, and third rounds of panning. 95 individual colonies from the second round of panning were randomly selected and analyzed by ELISA for the presence of LcrV-specific SAbs in their periplasmic extracts (not shown). Out of these 95 colonies, 85 scored positive. The VHHs from the 85 positive colonies were subjected to restriction fragment length polymorphism (RFLP) analysis using HinfI enzyme (not shown). Based on RFLP analysis, 40 colonies (several from each RFLP group) were selected for sequencing. Sequence analysis identified four different SAbs.

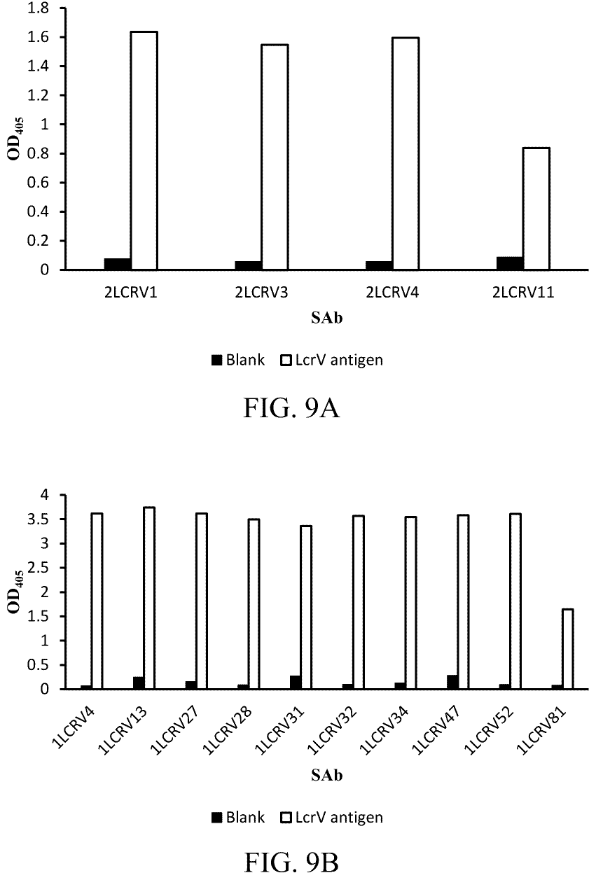

The high redundancy of the LcrV positive colonies identified after the second round of panning, together with the fact that the enrichment for antigen-specific phages was already good after the first round of panning, suggested that additional rounds of panning may have led to a loss of library diversity. To address this possibility and to identify additional unique sequences, 95 colonies from first round of panning were randomly selected and analyzed by ELISA for the presence of LcrV-specific SAbs in their periplasmic extracts, which is shown in FIGS. 9A-B. Out of these 95 colonies from the first round, 35 colonies were positive. These 35 colonies represented the four previously identified SAbs, as well as 10 novel sequences. The protein sequences of 14 exemplary LcrV SAbs according to the present invention are shown in Table 7, and the nucleic acid sequences encoding the 14 LcrV SAbs are shown in Table 8.

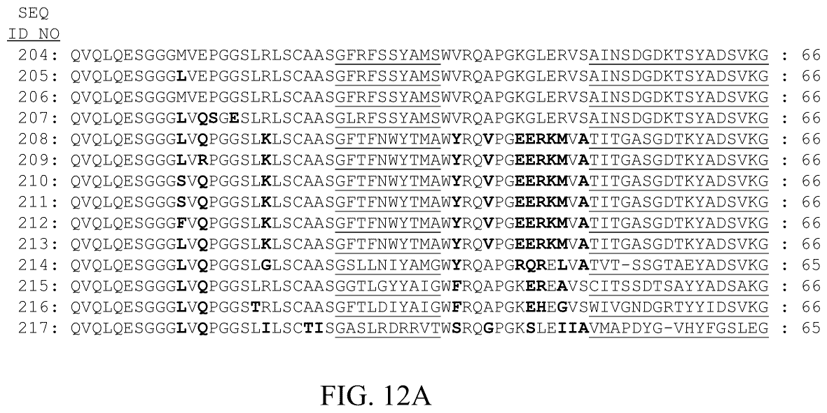

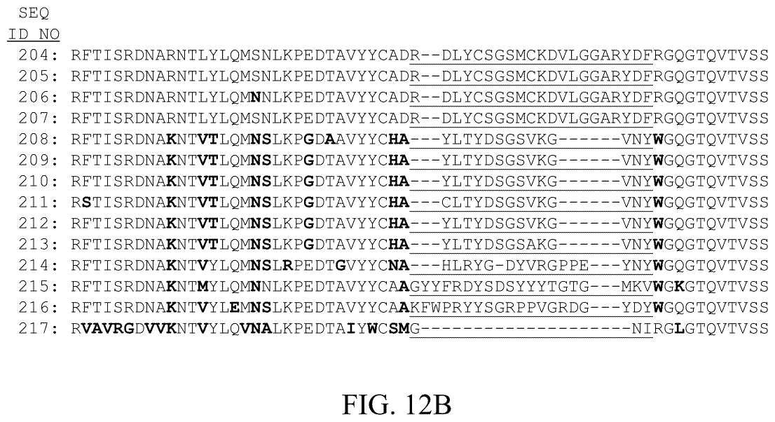

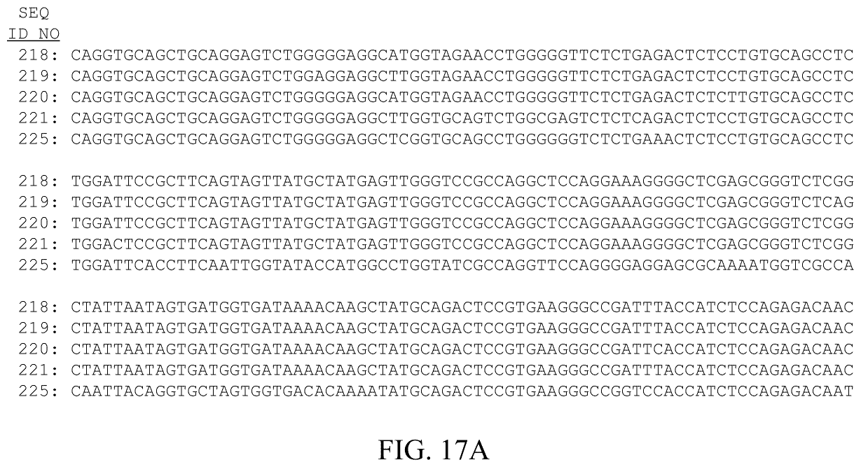

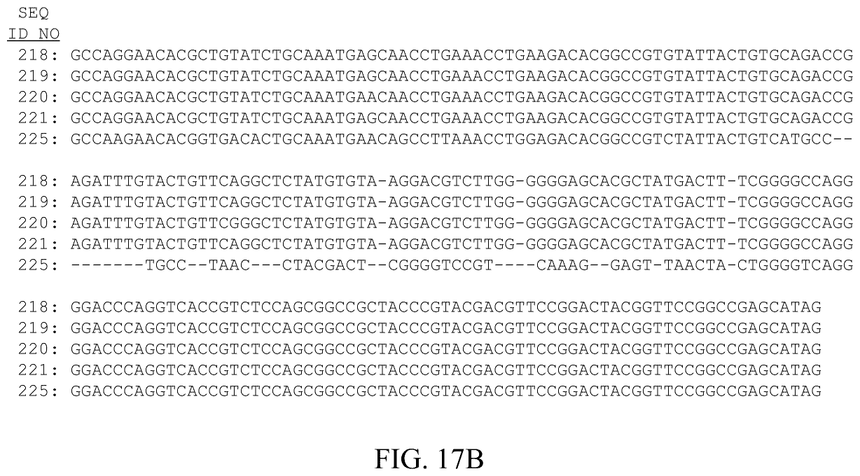

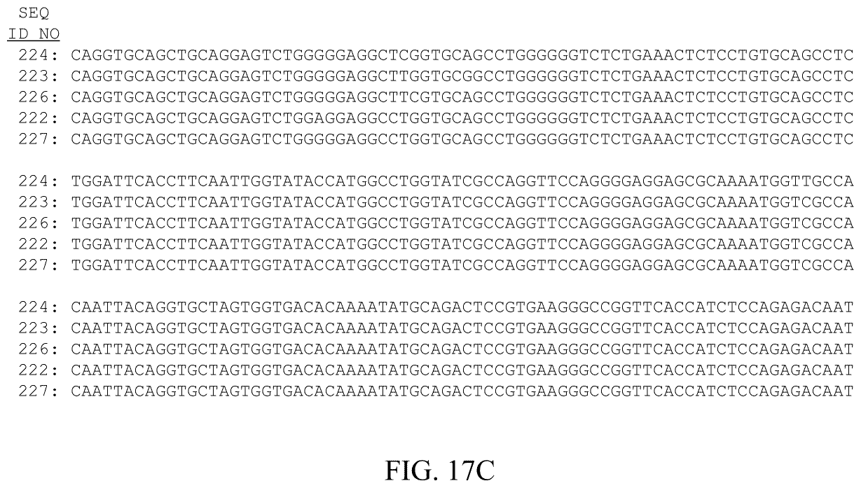

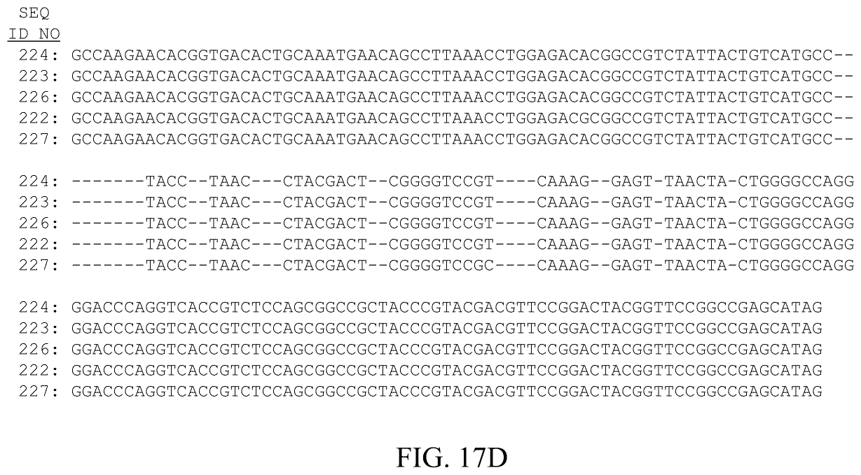

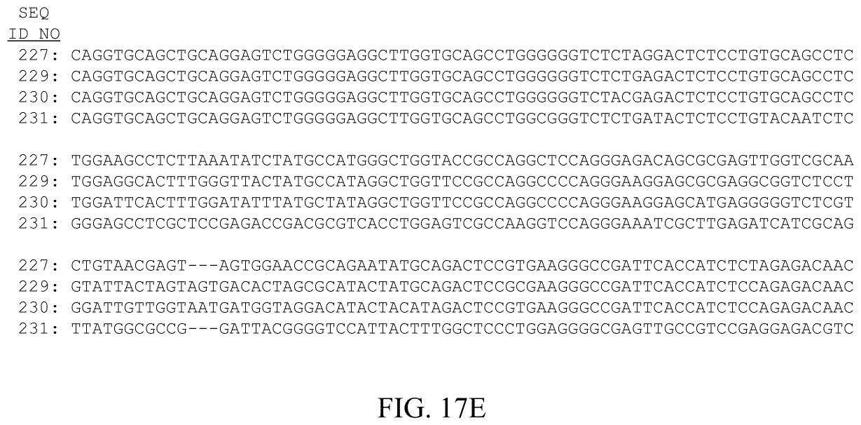

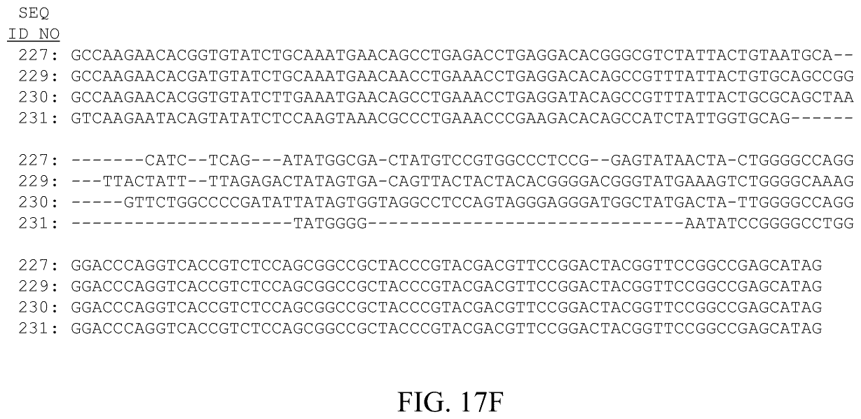

FIGS. 12A-B are the protein sequence alignment of the 14 exemplary LcrV SAbs listed in Table 7, and FIGS. 17A-F are sequence alignments of the nucleic acid sequences listed in Table 8. Gaps are introduced in the sequences in FIGS. 12A-B and 17A-F as needed in order to align the protein and nucleic acid sequences with one another. Referring to FIGS. 12A-B, the three CDRs are underlined in each sequence. The CDRs are defined according to the Kabat numbering system. The differences in the four FRs of each SAb (if any), as compared with 1LCRV32 (SEQ ID NO:204), are shown in bold; any differences between the three CDRs of each SAb are not otherwise indicated. The 14 exemplary LcrV SAbs shown in FIGS. 12A-B and listed in Table 7 represent six different groups, which are listed in Table 10. SAbs belonging to the same group are very similar, and their amino acid sequences suggest that they are from clonally-related B-cells resulting from somatic hypermutation or from the same B-cell with diversification due to PCR error during library construction.

### EXAMPLE 5

### Binding Kinetics of LcrV and F1 SAbs

Binding kinetics studies were conducted on selected LcrV and F1 SAbs. LcrV and F1 protein was immobilized on the surface of a BIACORE CM5 chip (GE Healthcare Biosciences), and each SAb was allowed to associate/dissociate with the appropriate antigen. The results of the binding kinetics study are shown in Table 11. Binding generally ranged from nM to pM, with the best two SAbs (LcrV-reactive SAbs SEQ ID NOs:209, 214) binding to the target in the mid-fM range. The binding constants of the seven LcrV SAbs from Table 11 (SEQ ID NOs:204, 209, 211, 214-217) are shown in Table 12. The K_{D} is calculated as k_{d}/kₐ (“n.b.”=no binding).

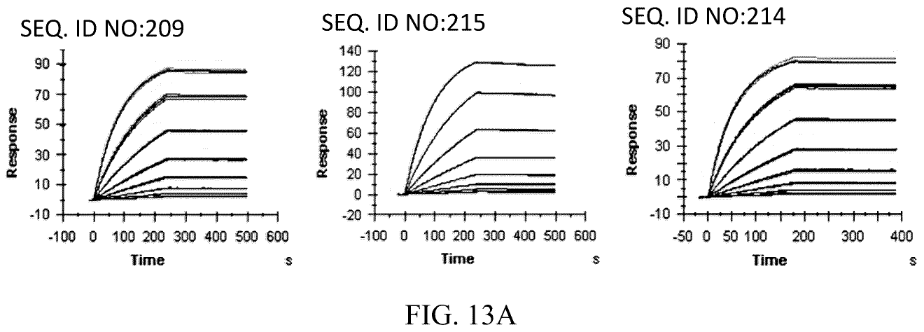

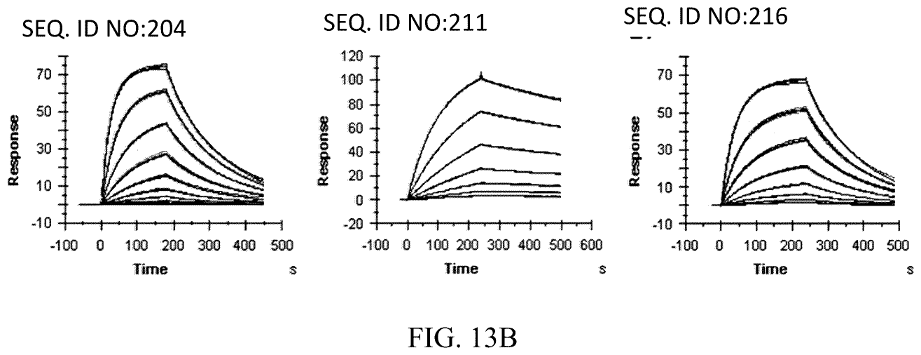

FIGS. 13A-B are the resulting double-referenced sensorgrams (colored by SAb concentration) obtained using a BIACORE T200 sensor instrument (General Electric Healthcare, United Kingdom) for six of the seven LcrV SAbs from Tables 11 and 12 (SEQ ID NOs:204, 209, 211, 214-217). A dissociation phase of 500 seconds was used for all concentrations of SAb. The overlaying curve fits are depicted in black, and the sensorgrams are based on a 1:1 binding model.

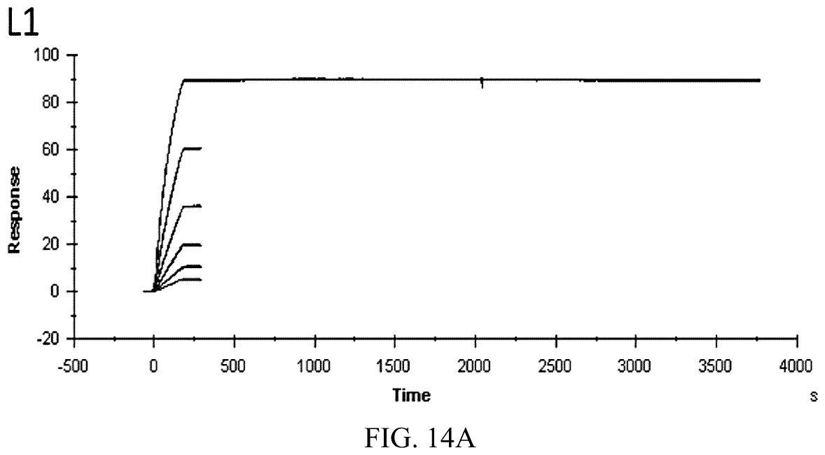

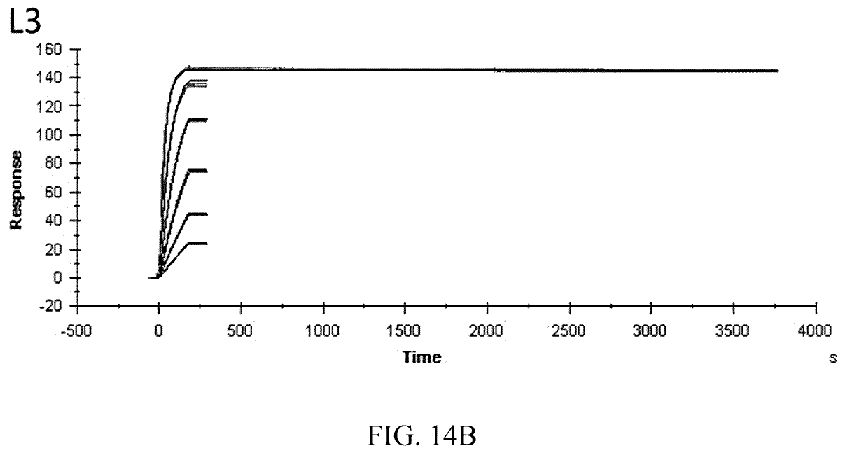

Of the described SAb sets, two SAbs (SEQ ID NOs:209, 214) demonstrate no discernible off rate (k_{d}) within the limits of THE BIACORE instrument analyses (see Tables 11 and 12). In a second test, LcrV was immobilized on the surface of a BIACORE CM5 chip, and LcrV-reactive SAbs SEQ ID NOs:209 and 214 were allowed to associate/dissociate. A dissociation phase of 120 seconds was used for all concentrations of SAb except the highest concentration, for which a 3600 second dissociation was used. FIGS. 14A-B are the double-referenced sensorgrams (colored by SAb concentration) obtained on the BIACORE T200 sensor instrument with overlaying curve fits (black), based on a 1:1 binding model. These data indicate that the SAb sequences of SEQ ID NOs:209 and 214 bind to the Y. pestis LcrV protein extremely and unusually tightly. Due to the nature of these two SAbs, both could bind to the Y. pestis bacteria in a manner that may make infection and/or replication difficult or impossible.

The present invention includes SAbs against at least one Y. pestis surface protein or antigen and the nucleotide sequences that encode the SAbs. The Y. pestis surface protein may include YscF, F1, and/or LcrV. The present invention includes a composition comprising a single SAb or a mixture of two or more different SAbs. For compositions comprising a mixture of two or more different SAbs, all of the SAbs may be against a single Y. pestis surface protein (single-antigen), or the SAbs may be against different epitopes on the same Y. pestis surface protein (single-antigen, multi-epitope). The mixture of two or more different SAbs may further comprise SAbs against two or more Y. pestis surface proteins (multi-antigen).

In one embodiment of the present invention, SAbs against at least one Y. pestis YscF epitope may comprise one each of a CDR1 sequence selected from the group consisting of SEQ ID NOs:1-7; a CDR2 sequence selected from the group consisting of SEQ ID NOs:27-33; and a CDR3 sequence selected from the group consisting of SEQ ID NOs:54-60. In another embodiment, SAbs against at least one Y. pestis YscF epitope may comprise one each of an FR1 sequence selected from the group consisting of SEQ ID NOs:79-102; a CDR1 sequence selected from the group consisting of SEQ ID NOs:1-7; an FR2 sequence selected from the group consisting of SEQ ID NOs:103-120; a CDR2 sequence selected from the group consisting of SEQ ID NOs:27-33; an FR3 sequence selected from the group consisting of SEQ ID NOs:121-146; a CDR3 sequence selected from the group consisting of SEQ ID NOs:54-60); and an FR4 sequence selected from the group consisting of SEQ ID NOs:147-153. In a further embodiment, SAbs against at least one Y. pestis YscF epitope may comprise the specific arrangement of FRs and CDRs embodied in SEQ ID NOs:154-160. The present invention further includes isolated nucleotide sequences selected from the group consisting of SEQ ID NOs:161-167 that encode the SAbs comprising SEQ ID NOs:154-160.

In another embodiment, SAbs against at least one Y. pestis F1 epitope may comprise one each of a CDR1 sequence selected from the group consisting of SEQ ID NOs:8-19; a CDR2 sequence selected from the group consisting of SEQ ID NOs:34-47; and a CDR3 sequence selected from the group consisting of SEQ ID NOs:61-71, AEY, and PGY. In another embodiment, SAbs against at least one Y. pestis F1 epitope may comprise one each of an FR1 sequence selected from the group consisting of SEQ ID NOs:79-102; a CDR1 sequence selected from the group consisting of SEQ ID NOs:8-19; an FR2 sequence selected from the group consisting of SEQ ID NOs:103-120; a CDR2 sequence selected from the group consisting of SEQ ID NOs:34-47; an FR3 sequence selected from the group consisting of SEQ ID NOs:121-146; a CDR3 sequence selected from the group consisting of SEQ ID NOs:61-71, AEY, and PGY; and an FR4 sequence selected from the group consisting of SEQ ID NOs:147-153. In a further embodiment, SAbs against at least one Y. pestis F1 epitope comprise the specific arrangement of FRs and CDRs embodied in SEQ ID NOs:168-185. The present invention further includes isolated nucleotide sequences selected from the group consisting of SEQ ID NOs:186-203 that encode the SAbs comprising SEQ ID NOs:168-185.

In a further embodiment, SAbs against at least one Y. pestis LcrV epitope may comprise one each of a CDR1 sequence selected from the group consisting of SEQ ID NOs:20-26; a CDR2 sequence selected from the group consisting of SEQ ID NOs:48-53; and a CDR3 sequence selected from the group consisting of SEQ ID NOs:72-78 and GNI. In another embodiment, SAbs against at least one Y. pestis LcrV epitope may comprise one each of an FR1 sequence selected from the group consisting of SEQ ID NOs:79-102; a CDR1 sequence selected from the group consisting of SEQ ID NOs:20-26; an FR2 sequence selected from the group consisting of SEQ ID NOs:103-120; a CDR2 sequence selected from the group consisting of SEQ ID NOs:48-53; an FR3 sequence selected from the group consisting of SEQ ID NOs:121-146; a CDR3 sequence selected from the group consisting of SEQ ID NOs:72-78 and GNI; and an FR4 sequence selected from the group consisting of SEQ ID NOs:147-153. In a further embodiment, SAbs against at least one Y. pestis LcrV epitope may comprise the specific arrangement of FRs and CDRs embodied in SEQ ID NOs:204-217. The present invention further includes isolated nucleotide sequences selected from the group consisting of SEQ ID NOs:218-231 that encode the SAbs comprising SEQ ID NOs:204-217.

In an alternative embodiment, the present invention includes one or more SAbs against Y. pestis YscF, with each SAb comprising a CDR1 sequence, a CDR2 sequence, and a CDR3 sequence respectively having at least 15% sequence identity with a CDR1 sequence selected from the group consisting of SEQ ID NOs:1-7; a CDR2 sequence selected from the group consisting of SEQ ID NOs:27-33; and a CDR3 sequence selected from the group consisting of SEQ ID NOs:54-60, in which the SAbs retain sufficient affinity for at least one of a Y. pestis YscF antigen or a Y. pestis YscF epitope. The present invention further includes one or more SAbs against Y. pestis YscF having at least 15% sequence identity with SEQ ID NOs:154-160, in which the SAbs retain sufficient affinity for at least one of a Y. pestis YscF antigen or a Y. pestis YscF epitope.

The present invention further includes one or more SAbs against Y. pestis F1, with each SAb comprising a CDR1 sequence, a CDR2 sequence, and a CDR3 sequence respectively having at least 15% sequence identity with at least one of a CDR1 sequence selected from the group consisting of SEQ ID NOs:8-19, a CDR2 sequence selected from the group consisting of SEQ ID NOs:34-47, and a CDR3 sequence selected from the group consisting of SEQ ID NOs:61-71, AEY, and PGY, in which the SAbs retain sufficient affinity for at least one of a Y. pestis F1 antigen or a Y. pestis F1 epitope. The present invention further includes one or more SAbs against Y. pestis F1 having at least 15% sequence identity with SEQ ID NOs:168-185, in which the SAbs retain sufficient affinity for at least one of a Y. pestis F1 antigen or a Y. pestis F1 epitope

The present invention further includes one or more SAbs against Y. pestis LcrV, with each SAb comprising a CDR1 sequence, a CDR2 sequence, and a CDR3 sequence respectively having at least 15% sequence identity with at least one of a CDR1 sequence selected from the group consisting of SEQ ID NOs:20-26, a CDR2 sequence selected from the group consisting of SEQ ID NOs:48-53, and a CDR3 sequence selected from the group consisting of SEQ ID NOs:72-78 and GNI, in which the SAbs retain sufficient affinity for at least one of a Y. pestis LcrV antigen or a Y. pestis LcrV epitope. The present invention further includes one or more SAbs against Y. pestis LcrV having at least 15% sequence identity with SEQ ID NOs:204-217, in which the SAbs retain sufficient affinity for at least one of a Y. pestis LcrV antigen or a Y. pestis LcrV epitope.

In an another embodiment, the present invention further includes a polypeptide, which is used herein to refer to a structure comprising two or more of any of the above-described SAbs against Y. pestis YscF, F1, and/or LcrV in which the two or more SAbs are joined together. In one embodiment, the polypeptide may comprise a fusion protein that is created by joining together two or more SAbs at the genetic level. Two or more nucleic acid sequences encoding for two or more SAbs may be spliced together, and translation of the spliced nucleic acid sequence creates a longer, multi-antigen and/or multi-epitope fusion protein. The fusion protein may contain up to four SAbs joined end-to-end in a substantially linear fashion, similar to beads on a string.

In one embodiment, the fusion protein comprises SAbs that are all against a single Y. pestis surface protein or antigen i.e. a single-antigen fusion protein against either YscF, F1, or LcrV. In a further embodiment, this single-antigen fusion protein further comprises SAbs that bind to two or more different epitopes (multi-epitope, single-antigen) on the single antigen. In another embodiment, the fusion protein may comprise SAbs against two or more different Y. pestis surface proteins i.e. a multi-antigen fusion protein. The multi-antigen fusion protein may also comprise SAbs that bind to two or more different epitopes (multi-epitope, multi-antigen) on the same antigen(s). In use, each individual fusion protein molecule may bind to one Y. pestis surface protein molecule, or the individual fusion protein molecule may be bound to two or more separate Y. pestis surface protein molecules. Use of a multi-antigen and/or multi-epitope fusion protein may increase avidity in enzyme immunosorbent assays.

In another embodiment, the polypeptide may be created by joining two or more SAbs together with a protein or chemical linker to create a multivalent protein complex. For example, a linker molecule such as the verotoxin 1B-subunit may be used to create high avidity, pentavalent SAb complexes similar to keys on a key ring. In one embodiment, the multivalent protein complex may contain SAbs that are all against a single Y. pestis surface protein or antigen i.e. a single-antigen multivalent protein complex. This single-antigen multivalent protein complex may further comprise SAbs that bind to two or more different epitopes (multi-epitope, single-antigen) on the single antigen. In another embodiment, the multivalent protein complex may comprise SAbs against two or more different Y. pestis surface proteins i.e. a multi-antigen multivalent protein complex. The multi-antigen multivalent protein complex may further comprise SAbs that bind to two or more different epitopes (multi-epitope, multi-antigen) on the same antigen. In use, each multivalent protein complex may bind to one Y. pestis surface protein molecule, or the multivalent protein complex may be bound to two or more separate Y. pestis surface protein molecules. These multi-antigen and/or multi-epitope multivalent protein complexes may generally demonstrate increased affinity for their respective epitope and/or antigen target(s) and may have numerous applications for biomarker assays or proteomics.

In one embodiment of the present invention, polypeptides as described herein comprise at least two SAbs, with the SAbs being selected from the following groups: (1) SAbs comprising one each of a CDR1 sequence selected from the group consisting of SEQ ID NOs:1-7; a CDR2 sequence selected from the group consisting of SEQ ID NOs:27-33; and a CDR3 sequence selected from the group consisting of SEQ ID NOs:54-60; (2) SAbs comprising one each of a CDR1 sequence selected from the group consisting of SEQ ID NOs:8-19; a CDR2 sequence selected from the group consisting of SEQ ID NOs:34-47; and a CDR3 sequence selected from the group consisting of SEQ ID NOs:61-71, AEY, and PGY; and (3) SAbs comprising one each of a CDR1 sequence selected from the group consisting of SEQ ID NOs:20-26; a CDR2 sequence selected from the group consisting of SEQ ID NOs:48-53; and a CDR3 sequence selected from the group consisting of SEQ ID NOs:72-78 and GNI.

In a further embodiment, the polypeptides comprise at least two SAbs selected from the group consisting of: (1) SAbs comprising one each of a CDR1 sequence selected from the group consisting of sequences having at least 15% sequence identity with SEQ ID NOs:1-7; a CDR2 sequence selected from the group consisting of sequences having at least 15% sequence identity with SEQ ID NOs:27-33; and a CDR3 sequence selected from the group consisting of sequences having at least 15% sequence identity with SEQ ID NOs:54-60; (2) SAbs comprising one each of a CDR1 sequence selected from the group consisting of sequences having at least 15% sequence identity with SEQ ID NOs:8-19; a CDR2 sequence selected from the group consisting of sequences having at least 15% sequence identity with SEQ ID NOs:34-47; and a CDR3 sequence selected from the group consisting of sequences having at least 15% sequence identity with SEQ ID NOs:61-71; and (3) SAbs comprising one each of a CDR1 sequence selected from the group consisting of sequences having at least 15% sequence identity with SEQ ID NOs:20-26; a CDR2 sequence selected from the group consisting of sequences having at least 15% sequence identity with SEQ ID NOs:48-53; and a CDR3 sequence selected from the group consisting of sequences having at least 15% sequence identity with SEQ ID NOs:72-78.

In another embodiment, the polypeptides may comprise at least two SAbs, with the SAbs being selected from the following groups: (1) SAbs comprising one set of CDR1, CDR2, and CDR3 sequences (as described above with respect to polypeptides according to the present invention) and one each of an FR1 sequence selected from the group consisting of SEQ ID NOs:79-102, an FR2 sequence selected from the group consisting of SEQ ID NOs:103-120, an FR3 sequence selected from the group consisting of SEQ ID NOs:121-146, and an FR4 sequence selected from the group consisting of SEQ ID NOs:147-153; and (2) SAbs selected from the group consisting of SEQ ID NOs:154-160, 168-185, and 204-217 and sequences having at least 15% sequence identity with SEQ ID NOs:154-160, 168-185, and 204-217.

In another embodiment, any of the SAbs or polypeptides according to the present invention may further comprise a protein tag, a protein domain tag, or a chemical tag. These tags generally comprise one or more additional amino acids or chemical molecules or residues that may be placed using known methods on the C- or N-terminus of the SAb or polypeptide without altering the activity or functionality of the SAb or polypeptide. The tag may facilitate purification of the SAb or polypeptide, direct absorption and/or excretion in the body, and/or facilitate use in a variety of applications such as detecting and monitoring Y. pestis. The tag may include, but is not limited to, a histidine tag (HIS tag) and a poly-lysine tag.

The present invention further includes a method of preventing or treating a Y. pestis infection in a patient. Y. pestis infections are frequently difficult to properly diagnose, which can result in delayed treatment, and a low toxicity treatment such as the presently disclosed SAbs may provide a valuable tool for cases of suspected Y. pestis exposure and/or infection and/or for patients presenting with ambiguous symptoms. The method comprises identifying a patient who is suspected of having been exposed to and/or infected with Y. pestis, and administering to the patient a pharmaceutically active amount of one or more of the SAbs and/or polypeptides according to the present invention. As used throughout, a “pharmaceutically active amount” refers generally to an amount that upon administration to the patient, is capable of providing directly or indirectly, one or more of the effects or activities disclosed herein. In one embodiment, the SAb(s) and/or polypeptide(s) may be administered as a form of passive immunotherapy in which the SAb(s) and/or polypeptide(s) are administered to the patient prior to at least one of exposure to or infection with Y. pestis. In another embodiment, the SAb(s) and/or polypeptide(s) may be administered after the patient is exposed to or infected with Y. pestis. The SAb(s) and/or polypeptide(s). In all embodiments of the methods, the SAb(s) and/or polypeptide(s) may be capable of being self-administered and may be administered to the patient using known techniques including, but not limited to, intravenous and subcutaneous injection, oral ingestion, inhalation, and topical administration. The ability to self-administer the SAb(s) and/or polypeptide(s) may be particularly useful in the case of an outbreak or attack where access to medical personnel and treatment may be limited.

The present invention further includes a method of detecting and/or diagnosing a Y. pestis infection using one of more of the SAbs and/or polypeptides herein described. The method may include detection of Y. pestis and diagnosis of the infection using known in vivo and/or in vitro assays such as enzyme linked immunosorbent assays (ELISAs), dot blot assays, and other suitable immunoassays. The Y. pestis SAb(s) and/or polypeptide(s) may, for example, be used as a primary antibody or a capture antibody in an ELISA for the detection/diagnosis of a Y. pestis infection. The SAb(s) and/or polypeptide(s) according to the present invention may further be coupled to one or more enzymes or markers for use in imaging.

The present invention further includes devices and methods for the identification and detection of Y. pestis on a surface and/or in an environment. A device for the environmental detection and/or quantification of Y. pestis may comprise one or more of the SAbs or polypeptides according to the present invention, with the SAb(s) and/or polypeptide(s) being used as a capture element. A method of identifying and detecting Y. pestis using the device comprises contacting one or more of the SAbs or polypeptides with an unknown target and detecting binding between the SAbs or polypeptides and the unknown target to identify the unknown target as Y. pestis. The method may further comprise use of the device to quantify an amount of Y. pestis on the surface and/or in the environment.

TABLE 1
Amino Acid Code

| | | | | | |
|---|---|---|---|---|---|
| Alanine | Ala | A | Methionine | Met | M |
| Cysteine | Cys | C | Asparagine | Asn | N |
| Aspartic Acid | Asp | D | Proline | Pro | P |
| Glutamic Acid | Glu | E | Glutamine | Gln | Q |
| Phenylalanine | Phe | F | Arginine | Arg | R |
| Glycine | Gly | G | Serine | Ser | S |
| Histidine | His | H | Threonine | Thr | T |
| Isoleucine | Ile | I | Valine | Val | V |
| Lysine | Lys | K | Tryptophan | Trp | W |
| Leucine | Leu | L | Tyrosine | Tyr | Y |

TABLE 2
Exemplary Combinations of FR and CDR Sequences

| | | | | | | | | | | | | | |
|---|---|---|---|---|---|---|---|---|---|---|---|---|---|
| ID # | FR1 | ID # | CDR1 | ID # | FR2 | ID # | CDR2 | ID # | FR3 | ID # | CDR3 | ID # | FR4 |

YscF SAb Sequences

| | | | | | | | | | | | | | |
|---|---|---|---|---|---|---|---|---|---|---|---|---|---|
| 79 | QVQLQESGG | 1 | GRTWR | 103 | WFRQ | 27 | VMSRSG | 121 | RFTISRDNAKN | 54 | GGGMY | 147 | WGKGTQ |
| | GLVQAGGSL | | AYYMG | | APGKE | | GTTSYA | | TVYLQMNNLA | | GPDLYG | | VTVSS |
| | RLSCAAS | | | | REFVA | | DSVKG | | PEDTATYYCK | | MTY | | |
| | | | | | | | | | A | | | | |
| 80 | QVQLQESGG | 2 | GRAFS | 103 | WFRQ | 28 | ANWRSG | 122 | RFTISRDDAKN | 55 | GGGSRW | 148 | WGQGTQ |
| | GLVQAGGSL | | NYAMA | | APGKE | | GLTDYA | | TVYLQMNSLK | | YGRTTA | | VTVSS |
| | RLSCVAS | | | | REFVA | | DSVKG | | PEDTAVYYCA | | SWYDY | | |
| | | | | | | | | | A | | | | |
| 81 | QVQLQESGG | 3 | GRTFSR | 103 | WFRQ | 29 | AISWSGS | 123 | RFTISRDHAKN | 56 | PAYGLR | 149 | RGQGTQ |
| | GLVQAGGSL | | YAMG | | APGKE | | STYYAD | | VMYLQMNGL | | PPYNY | | VTVSS |
| | RLSCAVS | | | | REFVA | | SVKG | | KPEDTGVYVC | | | | |
| | | | | | | | | | AR | | | | |
| 82 | QVQLQESGG | 4 | QRTFSR | 104 | WFRQ | 30 | ATTWSG | 124 | RFTISRDNAKN | 57 | GRSSWF | 150 | WGRGTQ |
| | GLVQAGGSL | | YSLG | | APGEE | | ISSDYAD | | TGYLQMNNLK | | APWLTP | | VTVSS |
| | KLSCTAS | | | | RVFVA | | SVKG | | PEDTGVYYCA | | YEYDY | | |
| | | | | | | | | | A | | | | |
| 79 | QVQLQESGG | 5 | GRTFSS | 105 | WFRQ | 31 | AIRWNG | 125 | RFTISRDLAKN | 58 | GVYDY | 148 | WGQGTQ |
| | GLVQAGGSL | | HAMA | | GPGEE | | DNIHYS | | TLYLQMNSLK | | | | VTVSS |
| | RLSCAAS | | | | RQFLA | | DSAKG | | PEDTAVYYCA | | | | |
| | | | | | | | | | R | | | | |
| 83 | QVQLQESGG | 6 | GRTFG | 106 | WFRRA | 32 | GITRSGN | 126 | RFTISRDNAKN | 59 | DWGWR | 148 | WGQGTQ |
| | GLVQAGDSR | | RPFRYT | | PGKER | | NIYYSDS | | TVYLQMNSLK | | NY | | VTVSS |
| | ILSCTAS | | MG | | EFVG | | VKG | | PEDTAVYYCN | | | | |
| | | | | | | | | | A | | | | |
| 84 | QVQLQESGG | 7 | GETVD | 107 | WFRQA | 33 | CISGSDG | 127 | RFTISRDNVKN | 60 | EIYDRR | 148 | WGQGT |
| | GLVQAGGSL | | DLAIG | | PGKER | | STYYAD | | TVYLQMNSLK | | WYRND | | QVTVSS |
| | RLACAAS | | | | EEIS | | SLSG | | LEDTAVYYCY | | Y | | |
| | | | | | | | | | A | | | | |

F1 SAb Sequences

| | | | | | | | | | | | | | |
|---|---|---|---|---|---|---|---|---|---|---|---|---|---|
| 81 | QVQLQESGG | 8 | GMMYI | 108 | WYRQA | 34 | FVSSTGN | 128 | RFTISRDNAKN | 61 | YLGSRD | 148 | WGQGT |
| | GLVQAGGSL | | REAIR | | PGKQR | | PRYTDS | | TVYLQMNSLTP | | Y | | QVTVSS |
| | RLSCAVS | | | | EWVA | | VKG | | EDTAVYYCNT | | | | |
| 85 | QVQLQESGG | 9 | GMMYI | 108 | WYRQA | 35 | VVSSTG | 128 | RFTISRDNAKN | 61 | YLGSRD | 148 | WGQGT |
| | GLVQPGGSL | | RYTMR | | PGKQR | | NPHYAD | | TVYLQMNSLTP | | Y | | QVTVSS |
| | RLSCAVS | | | | EWVA | | SVKG | | EDTAVYYCNT | | | | |
| 86 | QVQLQESGG | 10 | GRAVN | 109 | WYRQA | 36 | FISVGGT | 129 | RFTVSRDNAKN | * | AEY | 148 | WGQGT |
| | GLVRPGGSL | | RYHMH | | PGKQR | | TNYAGS | | TLYLQMNSLKP | | | | QVTVSS |
| | RLSCAVS | | | | EWVT | | VKG | | EDTAVYYCNS | | | | |
| 87 | QVQLQESGG | 11 | GIIFSD | 108 | WYRQA | 37 | QITRSQN | 130 | RFTVSRDNAKN | 62 | YDGRRP | 148 | WGQGT |
| | GSVQPGGSL | | YALT | | PGKQR | | INYTGSV | | TVHLQMNSLK | | PY | | QVTVSS |
| | SLSCSAS | | | | EWVA | | KG | | PEDTAVYYCH | | | | |
| | | | | | | | | | A | | | | |
| 87 | QVQLQESGG | 11 | GIIFSD | 108 | WYRQA | 37 | QITRSQN | 130 | RFTVSRDNAKN | 63 | YDGRRR | 148 | WGQGT |
| | GSVQPGGSL | | YALTVV | | PGKQR | | INYTGSV | | TVHLQMNSLK | | TY | | QVTVSS |
| | SLSCSAS | | | | EWVA | | PEDTAVYYCH | | A | | | | |
| | | | | | | | KG | | | | | | |
| 88 | QVQLQESGG | 11 | GIIFSD | 108 | WYRQA | 37 | QITRSQN | 130 | RFTVSRDNAKN | 62 | YDGRRP | 148 | WGQGT |
| | GLVQPGGSL | | YALT | | PGKQR | | INYTGSV | | TVHLQMNSLK | | PY | | QVTVSS |
| | SLSCSAS | | | | EWVA | | KG | | PEDTAVYYCH | | | | |
| | | | | | | | | | A | | | | |
| 88 | QVQLQESGG | 11 | GIIFSD | 108 | WYRQA | 38 | QITRRQ | 130 | RFTVSRDNAKN | 64 | YDGRRS | 148 | WGQGTQ |
| | GLVQPGGSL | | YALT | | PGKQR | | NINYTG | | TVHLQMNSLKP | | PY | | VTVSS |
| | SLSCSAS | | | | EWVA | | SVKG | | EDTAVYYCHA | | | | |
| 89 | QVQLQESGG | 11 | GIIFSD | 108 | WYRQA | 37 | QITRSQN | 131 | RFTVSRDNAKN | 62 | YDGRRP | 148 | WGQGTQ |
| | GLVQPGGSL | | YALT | | PGKQR | | INYTGS | | TVHLQMNSLKP | | PY | | VTVSS |
| | RLSCSAS | | | | EWVA | | VKG | | EDAAVYYCHA | | | | |
| 90 | QVQLQESGG | 12 | ARIFSI | 108 | WYRQA | 39 | AITTGGT | 126 | RFTISRDNAKN | * | PGY | 148 | WGQGTQ |
| | GLVQPGGSL | | YAMV | | PGKQR | | TNYADS | | TVYLQMNSLKP | | | | VTVSS |
| | RLSCAAS | | | | EWVA | | VKG | | EDTAVYYCNA | | | | |
| 90 | QVQLQESGG | 13 | GVIASI | 110 | WYRQT | 40 | IITSGGN | 132 | RFTTSRDNARN | 65 | LVGAKD | 148 | WGQGTQ |
| | GLVQPGGSL | | SVLR | | PGKTR | | TRYADS | | TVYLQMNSLKP | | Y | | VTVSS |
| | RLSCAAS | | | | DWVA | | VKG | | EDTAVYYCNT | | | | |
| 91 | QVQLQESGG | 14 | GTTFRS | 111 | WYRQA | 41 | FISSPGD | 133 | RFTISRDNAKN | 66 | NGIY | 147 | WGKGTQ |
| | GLVRPGGSL | | | | PGKER | | RTRYTE | | ALYLQMNGLK | | | | VTVSS |
| | RLSCEAS | | LVMK | | EWVA | | AVKG | | PEDTAVYYCN | | | | |
| | | | | | | | | | A | | | | |
| 92 | QVQLQESGG | 15 | GFTFSN | 112 | WVRQA | 42 | TINSGG | 134 | RFTISRDNAKN | 67 | TASHIP | 151 | LSQGTQ |
| | GLVQSGDSL | | YAMS | | PGKGL | | GSTSYA | | TLYLQMNSLKP | | | | VTVSS |
| | RLSCAAS | | | | EWVS | | YSVKG | | EDTAVYYCAK | | | | |
| 90 | QVQLQESGG | 15 | GFTFSN | 112 | WVRQA | 43 | TINIGGG | 134 | RFTISRDNAKN | 67 | TASHIP | 151 | LSQGTQ |
| | GLVQPGGSL | | YAMS | | PGKGL | | STSYAD | | TLYLQMNSLKP | | | | VTVSS |
| | RLSCAAS | | | | EWVS | | SVKG | | EDTAVYYCAK | | | | |
| 90 | QVQLQESGG | 16 | GFTFRN | 112 | WVRQA | 44 | TINGGG | 135 | RFTISRDNAKN | 68 | TARDSR | 149 | RGQGTQ |
| | GLVQPGGSL | | YAMS | | PGKGL | | GITSYAD | | TMYLQMNSLK | | DS | | VTVSS |
| | RLSCAAS | | | | EWVS | | SVKG | | PEDTAVYYCA | | | | |
| | | | | | | | | | O | | | | |
| 90 | QVQLQESGG | 17 | GFTFSS | 113 | WVRLA | 45 | TINIAGG | 134 | RFTISRDNAKN | 69 | TAANWS | 149 | RGQGTQ |
| | GLVQPGGSL | | YAMS | | PGKGL | | ITSYADS | | TLYLQMNSLKP | | AQ | | VTVSS |
| | RLSCAAS | | | | EWVS | | VKG | | EDTAVYYCAK | | | | |
| 90 | QVQLQESGG | 17 | GFTFSS | 112 | WVRQA | 46 | TINMGG | 136 | RFTISRHNAKN | 70 | TAGNWS | 149 | RGQGTQ |
| | GLVQPGGSL | | YAMS | | PGKGL | | GTTSYA | | TLYLQMNSLKP | | AQ | | VTVSS |
| | RLSCAAS | | | | EWVS | | DSVKG | | EDTAVYYCAK | | | | |
| 90 | QVQLQESGG | 18 | GFTFST | 114 | WIRQPP | 47 | TITSAGG | 137 | RFTISRDNAKN | 71 | LVNLAQ | 152 | TGQGTQ |
| | GLVQPGGSL | | SAMS | | GKARE | | SISYVNS | | TLYLQMNMLK | | | | VTVSS |
| | RLSCAAS | | | | VVA | | VKG | | PEDTAVYYCAR | | | | |
| 90 | QVQLQESGG | 19 | GFTFST | 114 | WIRQPP | 47 | TITSAGG | 137 | RFTISRDNAKN | 71 | LVNLAQ | 152 | TGQGTQ |
| | GLVQPGGSL | | NAMS | | GKARE | | SISYVNS | | TLYLQMNMLK | | | | VTVSS |
| | RLSCAAS | | | | VVA | | VKG | | PEDTAVYYCAR | | | | |

LcrV SAb Sequences

| | | | | | | | | | | | | | |
|---|---|---|---|---|---|---|---|---|---|---|---|---|---|
| 93 | QVQLQESGG | 20 | GFRFSS | 115 | WVRQA | 48 | AINSDG | 138 | RFTISRDNARN | 72 | RDLYCS | 149 | RGQGTQ |
| | GMVEPGGSL | | YAMS | | PGKGL | | DKTSYA | | TLYLQMSNLKP | | GSMCKD | | VTVSS |
| | RLSCAAS | | | | ERVS | | DSVKG | | EDTAVYYCAD | | VLGGAR | | |
| | | | | | | | | | | | YDF | | |
| 94 | QVQLQESGG | 20 | GFRFSS | 115 | WVRQA | 48 | AINSDG | 138 | RFTISRDNARN | 72 | RDLYCS | 149 | RGQGTQ |
| | GLVEPGGSL | | YAMS | | PGKGL | | DKTSYA | | TLYLQMSNLKP | | GSMCKD | | VTVSS |
| | RLSCAAS | | | | ERVS | | DSVKG | | EDTAVYYCAD | | VLGGAR | | |
| | | | | | | | | | | | YDF | | |
| 93 | QVQLQESGG | 20 | GFRFSS | 115 | WVRQA | 48 | AINSDG | 139 | RFTISRDNARN | 72 | RDLYCS | 149 | RGQGTQ |
| | GMVEPGGSL | | YAMS | | PGKGL | | DKTSYA | | TLYLQMNNLK | | GSMCKD | | VTVSS |
| | RLSCAAS | | | | ERVS | | DSVKG | | PEDTAVYYCA | | VLGGAR | | |
| | | | | | | | | | D | | YDF | | |
| 95 | QVQLQESGG | 21 | GLRFSS | 115 | WVRQA | 48 | AINSDG | 138 | RFTISRDNARN | 72 | RDLYCS | 149 | RGQGTQ |
| | GLVQSGESL | | YAMS | | PGKGL | | DKTSYA | | TLYLQMSNLKP | | GSMCKD | | VTVSS |
| | RLSCAAS | | | | ERVS | | DSVKG | | EDTAVYYCAD | | VLGGAR | | |
| | | | | | | | | | | | YDF | | |
| 96 | QVQLQESGG | 22 | GFTFN | 116 | WYRQV | 49 | TITGASG | 140 | RFTISRDNAKN | 73 | YLTYDS | 148 | WGQGTQ |
| | GLVQPGGSL | | WYTM | | PGEER | | DTKYAD | | TVTLQMNSLKP | | GSVKGV | | VTVSS |
| | KLSCAAS | | A | | KMVA | | SVKG | | GDAAVYYCHA | | NY | | |
| 97 | QVQLQESGG | 22 | GFTFN | 116 | WYRQV | 49 | TITGASG | 141 | RFTISRDNAKN | 73 | YLTYDS | 148 | WGQGTQ |
| | GLVRPGGSL | | WYTM | | PGEER | | DTKYAD | | TVTLQMNSLKP | | GSVKGV | | VTVSS |
| | KLSCAAS | | A | | KMVA | | SVKG | | GDTAVYYCHA | | NY | | |
| 98 | QVQLQESGG | 22 | GFTFN | 116 | WYRQV | 49 | TITGASG | 141 | RFTISRDNAKN | 73 | YLTYDS | 148 | WGQGTQ |
| | GSVQPGGSL | | WYTM | | PGEER | | DTKYAD | | TVTLQMNSLKP | | GSVKGV | | VTVSS |
| | KLSCAAS | | A | | KMVA | | SVKG | | GDTAVYYCHA | | NY | | |
| 98 | QVQLQESGG | 22 | GFTFN | 116 | WYRQV | 49 | TITGASG | 142 | RSTISRDNAKN | 74 | CLTYDS | 148 | WGQGTQ |
| | GSVQPGGSL | | WYTM | | PGEER | | DTKYAD | | TVTLQMNSLKP | | GSVKGV | | VTVSS |
| | KLSCAAS | | A | | KMVA | | SVKG | | GDTAVYYCHA | | NY | | |
| 99 | QVQLQESGG | 22 | GFTFN | 116 | WYRQV | 49 | TITGASG | 141 | RFTISRDNAKN | 73 | YLTYDS | 148 | WGQGTQ |
| | GFVQPGGSL | | WYTM | | PGEER | | DTKYAD | | TVTLQMNSLKP | | GSVKGV | | VTVSS |
| | KLSCAAS | | A | | KMVA | | SVKG | | GDTAVYYCHA | | NY | | |
| 96 | QVQLQESGG | 22 | GFTFN | 116 | WYRQV | 49 | TITGASG | 141 | RFTISRDNAKN | 75 | YLTYDS | 148 | WGQGTQ |
| | GLVQPGGSL | | WYTM | | PGEER | | DTKYAD | | TVTLQMNSLKP | | GSAKGV | | VTVSS |
| | KLSCAAS | | A | | KMVA | | SVKG | | GDTAVYYCHA | | NY | | |
| 100 | QVQLQESGG | 23 | GSLLNI | 117 | WYRQA | 50 | TVTSSG | 143 | RFTISRDNAKN | 76 | HLRYGD | 148 | WGQGTQ |
| | GLVQPGGSL | | YAMG | | PGRQR | | TAEYAD | | TVYLQMNSLRP | | YVRGPP | | VTVSS |
| | GLSCAAS | | | | ELVA | | SVKG | | EDTGVYYCNA | | EYNY | | |
| 90 | QVQLQESGG | 24 | GGTLG | 118 | WFRQA | 51 | CITSSDT | 144 | RFTISRDNAKN | 77 | GYYFRD | 147 | WGKGTQ |
| | GLVQPGGSL | | YYAIG | | PGKER | | SAYYAD | | TMYLQMNNLK | | YSDSYY | | VTVSS |
| | RLSCAAS | | | | EAVS | | SAKG | | PEDTAVYYCA | | YTGTGM | | |
| | | | | | | | | | A | | KV | | |
| 101 | QVQLQESGG | 25 | GFTLDI | 119 | WFRQA | 52 | WIVGND | 145 | RFTISRDNAKN | 78 | KFWPRY | 148 | WGQGTQ |
| | GLVQPGGST | | YAIG | | PGKEH | | GRTYYI | | TVYLEMNSLKP | | YSGRPP | | VTVSS |
| | RLSCAAS | | | | EGVS | | DSVKG | | EDTAVYYCAA | | VGRDGY | | |
| | | | | | | | | | | | DY | | |
| 102 | QVQLQESGG | 26 | GASLR | 120 | WSRQG | 53 | VMAPDY | 146 | RVAVRGDVVK | * | GNI | 153 | RGLGTQ |
| | GLVQPGGSL | | DRRVT | | PGKSLE | | GVHYFG | | NTVYLQVNAL | | | | VTVSS |
| | ILSCTIS | | | | IIA | | SLEG | | KPEDTAIYWCS | | | | |
| | | | | | | | | | M | | | | |

* These sequences have fewer than the required minimum of four amino acids and are not assigned a SEQ. NO.

TABLE 3
Y. pestis YscF SAb Protein Sequences

| SEQ ID | | |
|---|---|---|
| NO | Name | Sequence |
| 154 | 3yscf57 | QVQLQESGGGLVQAGGSLRLSCAASGRTWRAYYMGWFRQAPGKEREFVAVMSRSGGTTSYADSVK |
| | | GRFTISRDNAKNTVYLQMNNLAPEDTATYYCKAGGGMYGPDLYGMTYWGKGTQVTVSS |
| 155 | 3yscf124 | QVQLQESGGGLVQAGGSLRLSCVASGRAFSNYAMAWFRQAPGKEREFVAANWRSGGLTDYADSVK |
| | | GRFTISRDDAKNTVYLQMNSLKPEDTAVYYCAAGGGSRWYGRTTASWYDYWGQGTQVTVSS |
| 156 | 3yscf15 | QVQLQESGGGLVQAGGSLRLSCAVSGRTFSRYAMGWFRQAPGKEREFVAAISWSGSSTYYADSVKG |
| | | RFTISRDHAKNVMYLQMNGLKPEDTGVYVCARPAYGLRPPYNYRGQGTQVTVSS |
| 157 | 3yscf24 | QVQLQESGGGLVQAGGSLKLSCTASQRTFSRYSLGWFRQAPGEERVFVAATTWSGISSDYADSVKG |
| | | RFTISRDNAKNTGYLQMNNLKPEDTGVYYCAAGRSSWFAPWLTPYEYDYWGRGTQVTVSS |
| 158 | 3yscf142 | QVQLQESGGGLVQAGGSLRLSCAASGRTFSSHAMAWFRQGPGEERQFLAAIRWNGDNIHYSDSAKG |
| | | RFTISRDLAKNTLYLQMNSLKPEDTAVYYCARGVYDYWGQGTQVTVSS |
| 159 | 3yscf75 | QVQLQESGGGLVQAGDSRILSCTASGRTFGRPFRYTMGWFRRAPGKEREFVGGITRSGNNIYYSDSV |
| | | KGRFTISRDNAKNTVYLQMNSLKPEDTAVYYCNADWGWRNYWGQGTQVTVSS |
| 160 | 3yscf140 | QVQLQESGGGLVQAGGSLRLACAASGETVDDLAIGWFRQAPGKEREEISCISGSDGSTYYADSLSGRF |
| | | TISRDNVKNTVYLQMNSLKLEDTAVYYCYAEIYDRRWYRNDYWGQGTQVTVSS |

TABLE 4
Y. pestis YscF SAb DNA Sequences

| SEQ ID | | |
|---|---|---|
| NO | Name | Sequence |
| 161 | 3yscf57 | CAGGTGCAGCTGCAGGAGTCTGGGGGAGGATTGGTGCAGGCTGGGGGCTCTCTGAGACTCTCCT |
| | | GTGCAGCCTCTGGACGCACCTGGAGAGCCTATTACATGGGCTGGTTCCGCCAGGCTCCAGGGAA |
| | | GGAGCGTGAGTTTGTAGCAGTTATGAGTCGGAGCGGTGGCACCACATCCTATGCGGACTCCGTG |
| | | AAGGGCCGATTCACCATCTCCAGAGACAACGCCAAGAACACGGTGTATCTACAAATGAACAACC |
| | | TGGCACCTGAGGACACGGCCACGTATTATTGTAAGGCGGGGGGCGGAATGTACGGGCCGGACCT |
| | | GTATGGTATGACATACTGGGGCAAAGGGACCCAGGTCACCGTCTCCAGCGGCCGCTACCCGTAC |
| | | GACGTTCCGGACTACGGTTCCGGCCGAGCATAG |
| 162 | 3yscf124 | CAGGTGCAGCTGCAGGAGTCTGGAGGAGGATTGGTACAGGCTGGGGGCTCTCTGAGACTCTCCT |
| | | GTGTAGCCTCTGGACGCGCCTTCAGTAATTATGCGATGGCCTGGTTCCGCCAGGCTCCAGGGAAG |
| | | GAGCGTGAGTTTGTAGCAGCTAATTGGCGGAGTGGTGGTCTTACAGACTATGCAGACTCCGTGA |
| | | AGGGCCGATTCACCATCTCCAGAGACGACGCCAAGAACACGGTGTATCTGCAAATGAACAGCCT |
| | | GAAACCTGAGGACACGGCCGTTTATTACTGTGCCGCCGGGGGCGGTAGTCGCTGGTACGGGCGA |
| | | ACAACCGCAAGTTGGTATGACTACTGGGGCCAGGGGACCCAGGTCACCGTCTCCAGCGGCCGCT |
| | | ACCCGTACGACGTTCCGGACTACGGTTCCGGCCGAGCATAG |
| 163 | 3yscf15 | CAGGTGCAGCTGCAGGAGTCTGGGGGAGGATTGGTGCAGGCTGGGGGCTCTCTGAGACTCTCCT |
| | | GTGCAGTCTCTGGACGCACCTTCAGTAGATATGCCATGGGCTGGTTCCGCCAGGCTCCAGGGAAG |
| | | GAGCGTGAGTTTGTAGCAGCTATTAGCTGGAGTGGTAGTAGCACATATTATGCAGACTCCGTGAA |
| | | GGGCCGATTCACCATCTCCAGAGACCACGCCAAGAACGTGATGTATCTGCAAATGAACGGCCTG |
| | | AAACCTGAGGACACGGGTGTTTATGTCTGTGCAAGACCAGCGTACGGACTCCGCCCCCCGTATA |
| | | ATTACCGGGGCCAGGGGACCCAGGTCACCGTCTCCAGCGGCCGCTACCCGTACGACGTTCCGGA |
| | | CTACGGTTCCGGCCGAGCATAG |
| 164 | 3yscf24 | CAGGTGCAGCTGCAGGAGTCTGGGGGAGGATTGGTGCAGGCTGGGGGCTCTCTGAAACTCTCCT |
| | | GCACAGCCTCTCAACGCACCTTCAGTCGCTATAGCTTGGGCTGGTTCCGCCAGGCTCCAGGTGAG |
| | | GAGCGTGTTTTTGTAGCCGCTACTACATGGAGTGGTATAAGCAGTGACTATGCAGACTCCGTGAA |
| | | GGGCCGATTCACCATCTCCAGAGACAACGCCAAGAACACGGGGTATCTGCAAATGAACAATTTA |
| | | AAACCTGAGGACACGGGCGTTTATTACTGTGCAGCAGGACGTAGTAGCTGGTTCGCCCCCTGGTT |
| | | GACCCCCTATGAGTATGATTATTGGGGCCGGGGGACCCAGGTCACCGTCTCCAGCGGCCGCTACC |
| | | CGTACGACGTTCCGGACTACGGTTCCGGCCGAGCATAG |
| 165 | 3yscf142 | CAGGTGCAGCTGCAGGAGTCTGGGGGAGGATTGGTGCAGGCTGGGGGCTCTCTGAGACTCTCCT |
| | | GTGCAGCCTCTGGACGCACCTTCAGTAGCCATGCCATGGCCTGGTTCCGCCAGGGTCCAGGAGA |
| | | GGAGCGTCAGTTTCTAGCAGCTATTAGATGGAATGGTGATAACATACACTATTCAGACTCCGCGA |
| | | AGGGCCGATTCACCATCTCCAGAGACCTCGCCAAGAACACGCTCTATCTGCAAATGAACAGCCT |
| | | GAAACCTGAGGACACGGCCGTGTATTACTGTGCAAGGGGGGTGTATGACTACTGGGGCCAGGGG |
| | | ACCCAGGTCACCGTCTCCAGCGGCCGCTACCCGTACGACGTTCCGGACTACGGTTCCGGCCGAGC |
| | | ATAG |
| 166 | 3yscf75 | CAGGTGCAGCTGCAGGAGTCTGGGGGAGGATTGGTGCAGGCTGGGGACTCTCGGATACTCTCCT |
| | | GTACAGCCTCTGGACGCACCTTTGGACGCCCCTTCAGATATACCATGGGCTGGTTCCGCCGGGCT |
| | | CCAGGGAAGGAGCGTGAGTTTGTAGGAGGTATTACAAGAAGTGGTAATAATATATACTATTCAG |
| | | ACTCCGTGAAGGGCCGATTCACCATCTCCAGAGACAACGCCAAGAACACGGTGTATCTCCAAAT |
| | | GAACAGCCTGAAACCTGAGGACACGGCCGTGTATTATTGTAACGCAGATTGGGGGTGGAGGAAC |
| | | TACTGGGGCCAGGGGACCCAGGTCACCGTCTCCAGCGGCCGCTACCCGTACGACGTTCCGGACT |
| | | ACGGTTCCGGCCGAGCATAG |
| 167 | 3yscf140 | CAGGTGCAGCTGCAGGAGTCTGGGGGAGGCTTGGTGCAGGCTGGGGGGTCTCTGAGACTCGCCT |
| | | GTGCAGCCTCTGGAGAGACTGTCGATGATCTTGCCATCGGCTGGTTCCGCCAGGCCCCAGGGAA |
| | | GGAGCGTGAGGAGATTTCATGTATTAGTGGTAGTGATGGTAGCACATACTATGCAGACTCCCTGT |
| | | CGGGCCGATTCACCATCTCCAGGGACAACGTCAAGAACACGGTGTATCTGCAAATGAACAGCCT |
| | | GAAACTTGAGGACACGGCCGTCTATTACTGTTATGCAGAGATTTACGATAGACGCTGGTATCGGA |
| | | ACGACTACTGGGGCCAGGGGACCCAGGTCACCGTCTCCAGCGGCCGCTACCCGTACGACGTTCC |
| | | GGACTACGGTTCCGGCCGAGCATAG |

TABLE 5
Y. pestis F1 SAb Protein Sequences

| SEQ ID | | |
|---|---|---|
| NO | Name | Sequence |
| 168 | 3F55 | QVQLQESGGGLVQAGGSLRLSCAVSGMMYIREAIRWYRQAPGKQREWVAFVSSTGNPRYTDSVKG |
| | | RFTISRDNAKNTVYLQMNSLTPEDTAVYYCNTYLGSRDYWGQGTQVTVSS |
| 169 | 3F85 | QVQLQESGGGLVQPGGSLRLSCAVSGMMYIRYTMRWYRQAPGKQREWVAVVSSTGNPHYADSVK |
| | | GRFTISRDNAKNTVYLQMNSLTPEDTAVYYCNTYLGSRDYWGQGTQVTVSS |
| 170 | 3F44 | QVQLQESGGGLVRPGGSLRLSCAVSGRAVNRYHMHWYRQAPGKQREWVTFISVGGTTNYAGSVKG |
| | | RFTVSRDNAKNTLYLQMNSLKPEDTAVYYCNSAEYWGQGTQVTVSS |
| 171 | 4F34 | QVQLQESGGGSVQPGGSLSLSCSASGIIFSDYALTWYRQAPGKQREWVAQITRSQNINYTGSVKGRFT |
| | | VSRDNAKNTVHLQMNSLKPEDTAVYYCHAYDGRRPPYWGQGTQVTVSS |
| 172 | 4F6 | QVQLQESGGGSVQPGGSLSLSCSASGIIFSDYALTWYRQAPGKQREWVAQITRSQNINYTGSVKGRFT |
| | | VSRDNAKNTVHLQMNSLKPEDTAVYYCHAYDGRRRTYWGQGTQVTVSS |
| 173 | 4F1 | QVQLQESGGGLVQPGGSLSLSCSASGIIFSDYALTWYRQAPGKQREWVAQITRSQNINYTGSVKGRFT |
| | | VSRDNAKNTVHLQMNSLKPEDTAVYYCHAYDGRRPPYWGQGTQVTVSS |
| 174 | 3F31 | QVQLQESGGGLVQPGGSLSLSCSASGIIFSDYALTWYRQAPGKQREWVAQITRRQNINYTGSVKGRF |
| | | TVSRDNAKNTVHLQMNSLKPEDTAVYYCHAYDGRRSPYWGQGTQVTVSS |
| 175 | 3F61 | QVQLQESGGGLVQPGGSLRLSCSASGIIFSDYALTWYRQAPGKQREWVAQITRSQNINYTGSVKGRF |
| | | TVSRDNAKNTVHLQMNSLKPEDAAVYYCHAYDGRRPPYWGQGTQVTVSS |
| 176 | 4F27 | QVQLQESGGGLVQPGGSLRLSCAASARIFSIYAMVWYRQAPGKQREWVAAITTGGTTNYADSVKGR |
| | | FTISRDNAKNTVYLQMNSLKPEDTAVYYCNAPGYWGQGTQVTVSS |
| 177 | 3F26 | QVQLQESGGGLVQPGGSLRLSCAASGVIASISVLRWYRQTPGKTRDWVAIITSGGNTRYADSVKGRF |
| | | TTSRDNARNTVYLQMNSLKPEDTAVYYCNTLVGAKDYWGQGTQVTVSS |
| 178 | 4F59 | QVQLQESGGGLVRPGGSLRLSCEASGTTFRSLVMKWYRQAPGKEREWVAFISSPGDRTRYTEAVKG |
| | | RFTISRDNAKNALYLQMNGLKPEDTAVYYCNANGIYWGKGTQVTVSS |
| 179 | 3F5 | QVQLQESGGGLVQSGDSLRLSCAASGFTFSNYAMSWVRQAPGKGLEWVSTINSGGGSTSYAYSVKG |
| | | RFTISRDNAKNTLYLQMNSLKPEDTAVYYCAKTASHIPLSQGTQVTVSS |
| 180 | 4F57 | QVQLQESGGGLVQPGGSLRLSCAASGFTFSNYAMSWVRQAPGKGLEWVSTINIGGGSTSYADSVKG |
| | | RFTISRDNAKNTLYLQMNSLKPEDTAVYYCAKTASHIPLSQGTQVTVSS |
| 181 | 4F75 | QVQLQESGGGLVQPGGSLRLSCAASGFTFRNYAMSWVRQAPGKGLEWVSTINGGGGITSYADSVKG |
| | | RFTISRDNAKNTMYLQMNSLKPEDTAVYYCAQTARDSRDSRGQGTQVTVSS |
| 182 | 3F59 | QVQLQESGGGLVQPGGSLRLSCAASGFTFSSYAMSWVRLAPGKGLEWVSTINIAGGITSYADSVKGR |
| | | FTISRDNAKNTLYLQMNSLKPEDTAVYYCAKTAANWSAQRGQGTQVTVSS |
| 183 | 4F78 | QVQLQESGGGLVQPGGSLRLSCAASGFTFSSYAMSWVRQAPGKGLEWVSTINMGGGTTSYADSVKG |
| | | RFTISRHNAKNTLYLQMNSLKPEDTAVYYCAKTAGNWSAQRGQGTQVTVSS |
| 184 | 3F1 | QVQLQESGGGLVQPGGSLRLSCAASGFTFSTSAMSWIRQPPGKAREVVATITSAGGSISYVNSVKGRF |
| | | TISRDNAKNTLYLQMNMLKPEDTAVYYCARLVNLAQTGQGTQVTVSS |
| 185 | 3F65 | QVQLQESGGGLVQPGGSLRLSCAASGFTFSTNAMSWIRQPPGKAREVVATITSAGGSISYVNSVKGRF |
| | | TISRDNAKNTLYLQMNMLKPEDTAVYYCARLVNLAQTGQGTQVTVSS |

TABLE 6
Y. pestis F1 SAb DNA Sequences

| SEQ ID | | |
|---|---|---|
| NO | Name | Sequence |
| 186 | 3F55 | CAGGTGCAGCTGCAGGAGTCTGGGGGAGGCTTGGTGCAGGCTGGGGGCTCTCTGAGACTCTCCT |
| | | GTGCAGTTTCTGGAATGATGTACATTAGGGAGGCTATACGCTGGTACCGCCAGGCTCCAGGGAA |
| | | GCAGCGCGAGTGGGTCGCCTTTGTAAGTAGTACTGGTAATCCACGCTATACAGACTCCGTGAAG |
| | | GGCCGATTCACCATCTCCAGAGACAACGCCAAGAACACGGTGTATCTGCAAATGAACAGCCTGA |
| | | CACCTGAGGACACGGCCGTCTATTACTGTAATACATACTTGGGCTCGAGGGACTACTGGGGCCA |
| | | GGGGACCCAGGTCACCGTCTCCAGCGGCCGCTACCCGTACGACGTTCCGGACTACGGTTCCGGCC |
| | | GAGCATAG |
| 187 | 3F85 | CAGGTGCAGCTGCAGGAGTCTGGAGGAGGCTTGGTGCAGCCTGGGGGGTCTCTGAGACTCTCCT |
| | | GTGCAGTTTCTGGAATGATGTACATTAGGTACACTATGCGCTGGTACCGCCAGGCTCCAGGGAAG |
| | | CAGCGCGAGTGGGTCGCCGTTGTAAGTAGTACTGGTAATCCACACTATGCAGACTCCGTGAAGG |
| | | GCCGATTCACCATCTCCAGAGACAACGCCAAGAACACGGTGTATCTGCAAATGAACAGCCTGAC |
| | | ACCTGAGGACACGGCCGTCTATTACTGTAATACATACTTGGGCTCGAGGGACTACTGGGGCCAG |
| | | GGGACCCAGGTCACCGTCTCCAGCGGCCGCTACCCGTACGACGTTCCGGACTACGGTTCCGGCCG |
| | | AGCATAG |
| 188 | 3F44 | CAGGTGCAGCTGCAGGAGTCTGGGGGAGGCTTGGTGCGGCCTGGGGGGTCTCTGAGACTCTCCT |
| | | GTGCAGTCTCTGGAAGAGCCGTCAATAGGTATCACATGCACTGGTACCGCCAGGCTCCAGGGAA |
| | | GCAGCGCGAGTGGGTCACATTTATTAGTGTTGGTGGTACCACAAACTATGCAGGCTCCGTGAAG |
| | | GGCCGATTCACCGTCTCCCGAGACAACGCCAAAAACACGCTGTATCTGCAAATGAACAGCCTGA |
| | | AACCTGAGGACACGGCCGTCTATTACTGTAATTCAGCTGAATACTGGGGCCAGGGGACCCAGGT |
| | | CACCGTCTCCAGCGGCCGCTACCCGTACGACGTTCCGGACTACGGTTCCGGCCGAGCATAG |
| 189 | 4F34 | CAGGTGCAGCTGCAGGAGTCTGGGGGAGGCTCGGTGCAGCCTGGGGGGTCTCTGAGCCTCTCCT |
| | | GTTCAGCCTCTGGAATCATCTTCAGTGACTATGCCCTGACCTGGTACCGCCAGGCTCCAGGGAAG |
| | | CAGCGCGAGTGGGTTGCACAGATTACGCGAAGTCAAAATATAAATTATACAGGATCCGTGAAGG |
| | | GCCGATTCACCGTCTCCAGAGACAACGCCAAGAACACAGTGCATCTGCAAATGAACAGCCTGAA |
| | | ACCTGAGGACACGGCCGTCTACTATTGTCATGCATATGACGGTCGACGCCCACCCTACTGGGGCC |
| | | AGGGGACCCAGGTCACCGTCTCCAGCGGCCGCTACCCGTACGACGTTCCGGACTACGGTTCCGG |
| | | CCGAGCATAG |
| 190 | 4F6 | CAGGTGCAGCTGCAGGAGTCTGGGGGAGGCTCGGTGCAGCCTGGGGGGTCTCTGAGCCTCTCCT |
| | | GTTCAGCCTCTGGAATCATCTTCAGTGACTATGCCCTGACCTGGTACCGCCAGGCTCCAGGGAAG |
| | | CAGCGCGAGTGGGTTGCACAGATTACGCGAAGCCAAAATATAAATTATACAGGATCCGTGAAGG |
| | | GCCGATTCACCGTCTCCAGAGACAACGCCAAGAACACAGTGCATCTGCAAATGAACAGCCTGAA |
| | | ACCTGAGGACACGGCCGTCTACTATTGTCATGCATATGACGGTCGACGCCGAACCTACTGGGGCC |
| | | AGGGGACCCAGGTCACCGTCTCCAGCGGCCGCTACCCGTACGACGTTCCGGACTACGGTTCCGG |
| | | CCGAGCATAG |
| 191 | 4F1 | CAGGTGCAGCTGCAGGAGTCTGGGGGAGGCTTGGTGCAGCCTGGGGGGTCTCTGAGCCTCTCCT |
| | | GTTCAGCCTCTGGAATCATCTTCAGTGACTATGCCCTGACCTGGTACCGCCAGGCTCCAGGGAAG |
| | | CAGCGCGAGTGGGTTGCACAGATTACGCGAAGCCAAAATATAAATTATACAGGATCCGTGAAGG |
| | | GCCGATTCACCGTCTCCAGAGACAACGCCAAGAACACAGTGCATCTGCAAATGAACAGCCTGAA |
| | | ACCTGAGGACACGGCCGTCTACTATTGTCATGCATATGACGGTCGACGCCCACCCTACTGGGGCC |
| | | AGGGGACCCAGGTCACCGTCTCCAGCGGCCGCTACCCGTACGACGTTCCGGACTACGGTTCCGG |
| | | CCGAGCATAG |
| 192 | 3F31 | CAGGTGCAGCTGCAGGAGTCTGGGGGAGGCTTGGTGCAGCCTGGGGGGTCTCTGAGCCTCTCCT |
| | | GTTCAGCCTCTGGAATCATCTTCAGTGACTATGCCCTGACCTGGTACCGCCAGGCTCCAGGGAAG |
| | | CAGCGCGAGTGGGTTGCACAGATTACGCGAAGGCAAAATATAAATTATACAGGATCCGTGAAGG |
| | | GCCGATTCACCGTCTCCAGAGACAACGCCAAGAACACAGTGCATCTGCAAATGAACAGCCTGAA |
| | | ACCTGAGGACACGGCCGTCTACTATTGTCATGCATATGACGGTCGACGATCACCCTACTGGGGCC |
| | | AGGGGACCCAGGTCACCGTCTCCAGCGGCCGCTACCCGTACGACGTTCCGGACTACGGTTCCGG |
| | | CCGAGCATAG |
| 193 | 3F61 | CAGGTGCAGCTGCAGGAGTCTGGGGGAGGCTTGGTGCAGCCTGGGGGGTCTCTGAGACTCTCCT |
| | | GTTCAGCCTCTGGAATCATCTTCAGTGACTATGCCCTGACCTGGTACCGCCAGGCTCCAGGGAAG |
| | | CAGCGCGAGTGGGTTGCACAGATTACGCGAAGTCAAAATATAAATTATACAGGATCCGTGAAGG |
| | | GCCGATTCACCGTCTCCAGAGACAACGCCAAGAACACAGTGCATCTGCAAATGAACAGCCTGAA |
| | | ACCTGAGGACGCGGCCGTCTACTATTGTCATGCATATGACGGTCGACGCCCACCCTACTGGGGCC |
| | | AGGGGACCCAGGTCACCGTCTCCAGCGGCCGCTACCCGTACGACGTTCCGGACTACGGTTCCGG |
| | | CCGAGCATAG |
| 194 | 4F27 | CAGGTGCAGCTGCAGGAGTCTGGGGGAGGCTTGGTGCAGCCTGGGGGGTCTCTGAGACTCTCCT |
| | | GTGCAGCCTCTGCCCGCATCTTCAGTATCTATGCCATGGTATGGTACCGCCAGGCTCCAGGGAAG |
| | | CAGCGCGAGTGGGTCGCAGCTATTACTACTGGTGGTACCACAAACTATGCAGACTCCGTGAAGG |
| | | GCCGATTCACCATCTCCAGAGACAACGCCAAGAACACGGTGTATCTGCAAATGAACAGCCTGAA |
| | | ACCTGAGGACACGGCCGTCTATTACTGTAATGCTCCGGGCTACTGGGGCCAGGGGACCCAGGTC |
| | | ACCGTCTCCAGCGGCCGCTACCCGTACGACGTTCCGGACTACGGTTCCGGCCGAGCATAG |
| 195 | 3F26 | CAGGTGCAGCTGCAGGAGTCTGGGGGAGGCTTGGTGCAGCCTGGGGGGTCTCTGAGACTCTCCT |
| | | GTGCAGCCTCTGGAGTCATCGCCAGTATCTCCGTCCTGCGCTGGTACCGCCAAACACCAGGAAAG |
| | | ACGCGCGACTGGGTCGCAATTATTACTAGTGGTGGCAACACACGCTATGCAGACTCCGTGAAGG |
| | | GCCGATTCACCACCTCCAGAGATAACGCCAGGAACACGGTGTATCTGCAAATGAACAGCCTGAA |
| | | ACCTGAGGACACGGCCGTCTATTACTGTAATACACTTGTAGGAGCCAAGGACTACTGGGGCCAG |
| | | GGGACCCAGGTCACCGTCTCCAGCGGCCGCTACCCGTACGACGTTCCGGACTACGGTTCCGGCCG |
| | | AGCATAG |
| 196 | 4F59 | CAGGTGCAGCTGCAGGAGTCTGGGGGAGGCTTGGTGCGGCCTGGGGGATCTCTAAGACTCTCCT |
| | | GTGAAGCCTCTGGAACCACCTTCAGAAGCCTCGTAATGAAATGGTACCGCCAGGCTCCAGGGAA |
| | | GGAGCGCGAGTGGGTCGCATTTATTTCTAGTCCTGGTGATCGCACTCGCTACACAGAAGCCGTGA |
| | | AGGGCCGATTCACCATCTCCAGAGACAATGCCAAGAACGCGCTGTATCTGCAAATGAACGGCCT |
| | | GAAACCTGAGGACACGGCCGTGTATTATTGTAACGCGAACGGAATATACTGGGGCAAAGGGACC |
| | | CAGGTCACCGTCTCCAGCGGCCGCTACCCGTACGACGTTCCGGACTACGGTTCCGGCCGAGCATA |
| | | G |
| 197 | 3F5 | CAGGTGCAGCTGCAGGAGTCTGGGGGAGGCTTGGTGCAATCTGGGGATTCTCTGAGACTCTCCTG |
| | | TGCAGCCTCTGGATTCACCTTCAGTAACTATGCTATGAGCTGGGTCCGCCAGGCTCCAGGAAAGG |
| | | GGCTCGAGTGGGTCTCAACTATTAATAGTGGTGGTGGTAGCACAAGCTATGCGTACTCCGTGAAG |
| | | GGCCGATTCACCATCTCCAGAGACAACGCCAAGAACACGCTGTATCTGCAAATGAACAGCCTGA |
| | | AACCTGAGGACACGGCCGTGTATTACTGTGCAAAGACGGCCTCTCACATACCCTTGAGCCAGGG |
| | | GACCCAGGTCACCGTCTCCAGCGGCCGCTACCCGTACGACGTTCCGGACTACGGTTCCGGCCGAG |
| | | CATAG |
| 198 | 4F57 | CAGGTGCAGCTGCAGGAGTCTGGGGGAGGCTTGGTGCAACCTGGGGGTTCTCTGAGACTCTCCT |
| | | GTGCAGCCTCTGGATTCACCTTCAGTAACTATGCTATGAGCTGGGTCCGCCAGGCTCCAGGAAAG |
| | | GGGCTCGAGTGGGTCTCAACTATTAATATTGGTGGTGGTAGCACAAGCTATGCAGACTCCGTGAA |
| | | GGGCCGATTCACCATCTCCAGAGACAACGCCAAGAACACGCTGTATCTGCAAATGAACAGCCTG |
| | | AAACCTGAGGACACGGCCGTGTATTACTGTGCAAAGACGGCCTCTCACATACCCTTGAGCCAGG |
| | | GGACCCAGGTCACCGTCTCCAGCGGCCGCTACCCGTACGACGTTCCGGACTACGGTTCCGGCCGA |
| | | GCATAG |
| 199 | 4F75 | CAGGTGCAGCTGCAGGAGTCTGGGGGAGGCTTGGTGCAACCTGGGGGTTCTCTGAGACTCTCCT |
| | | GTGCAGCCTCTGGATTCACCTTCAGGAACTATGCAATGAGCTGGGTCCGTCAGGCTCCAGGAAA |
| | | GGGGCTCGAGTGGGTCTCAACTATTAATGGTGGTGGTGGTATCACAAGCTATGCAGACTCCGTGA |
| | | AGGGCCGATTCACCATCTCCAGAGACAACGCCAAGAACACAATGTATCTGCAAATGAACAGCCT |
| | | GAAACCTGAGGACACGGCCGTCTATTACTGTGCCCAAACCGCCCGCGATTCCCGCGATTCCCGGG |
| | | GCCAGGGGACCCAGGTCACCGTCTCCAGCGGCCGCTACCCGTACGACGTTCCGGACTACGGTTCC |
| | | GGCCGAGCATAG |
| 200 | 3F59 | CAGGTGCAGCTGCAGGAGTCTGGGGGAGGCTTGGTGCAACCTGGGGGTTCTCTGAGACTCTCCT |
| | | GTGCAGCCTCTGGATTCACCTTCAGTAGCTATGCTATGAGCTGGGTCCGCCTGGCTCCAGGAAAG |
| | | GGGCTCGAGTGGGTCTCAACTATTAATATCGCTGGTGGTATCACAAGCTATGCAGACTCCGTGAA |
| | | GGGCCGATTCACCATCTCCAGAGACAACGCCAAGAACACGCTGTATCTGCAAATGAACAGCCTG |
| | | AAACCTGAGGACACGGCCGTGTATTACTGTGCAAAAACGGCGGCCAACTGGAGCGCCCAGAGAG |
| | | GCCAGGGGACCCAGGTCACCGTCTCCAGCGGCCGCTACCCGTACGACGTTCCGGACTACGGTTCC |
| | | GGCCGAGCATAG |
| 201 | 4F78 | CAGGTGCAGCTGCAGGAGTCTGGGGGAGGCTTGGTGCAACCTGGGGGTTCTCTGAGACTCTCCT |
| | | GTGCAGCCTCTGGATTCACCTTCAGTAGCTATGCTATGAGCTGGGTCCGCCAGGCTCCAGGAAAG |
| | | GGGCTCGAGTGGGTCTCAACTATTAATATGGGTGGTGGTACCACAAGCTATGCAGACTCCGTGA |
| | | AGGGCCGATTCACCATCTCCAGACACAACGCCAAGAACACGCTGTATCTGCAAATGAACAGCCT |
| | | GAAACCTGAGGACACGGCCGTGTATTACTGTGCAAAAACGGCGGGCAACTGGAGCGCCCAGAG |
| | | AGGCCAGGGGACCCAGGTCACCGTCTCCAGCGGCCGCTACCCGTACGACGTTCCGGACTACGGT |
| | | TCCGGCCGAGCATAG |
| 202 | 3F1 | CAGGTGCAGCTGCAGGAGTCTGGGGGAGGCTTGGTGCAACCTGGGGGTTCTCTGAGACTGTCCT |
| | | GTGCAGCCTCTGGATTCACCTTCAGTACAAGTGCCATGAGTTGGATCCGCCAGCCTCCAGGGAAG |
| | | GCGCGCGAGGTGGTCGCAACTATTACTAGTGCTGGTGGTAGTATAAGTTATGTAAACTCCGTGAA |
| | | GGGCCGATTCACCATCTCCAGAGACAACGCCAAGAACACGCTGTATCTGCAAATGAACATGCTG |
| | | AAACCTGAGGACACGGCCGTGTATTACTGTGCCCGACTGGTCAACCTTGCCCAGACCGGCCAGG |
| | | GAACCCAGGTCACCGTCTCCAGCGGCCGCTACCCGTACGACGTTCCGGACTACGGTTCCGGCCGA |
| | | GCATAG |
| 203 | 3F65 | CAGGTGCAGCTGCAGGAGTCTGGGGGAGGCCTGGTGCAACCTGGGGGTTCTCTGAGACTGTCCT |
| | | GTGCAGCCTCTGGATTCACCTTCAGTACAAATGCCATGAGTTGGATCCGCCAGCCTCCAGGGAAG |
| | | GCGCGCGAGGTGGTCGCAACTATTACTAGTGCTGGTGGTAGTATAAGTTATGTAAACTCCGTGAA |
| | | GGGCCGATTCACCATCTCCAGAGACAACGCCAAGAACACGCTGTATCTGCAAATGAACATGCTG |
| | | AAACCTGAGGACACGGCCGTGTATTACTGTGCCCGACTGGTCAACCTTGCCCAGACCGGCCAGG |
| | | GGACCCAGGTCACCGTCTCCAGCGGCCGCTACCCGTACGACGTTCCGGACTACGGTTCCGGCCGA |
| | | GCATAG |

TABLE 7
Y. pestis LcrV SAb Protein Sequences

| SEQ ID | | |
|---|---|---|
| NO | Name | Sequence |
| 204 | 1LCRV32 | QVQLQESGGGMVEPGGSLRLSCAASGFRFSSYAMSWVRQAPGKGLERVSAINSDGDKTSYADSVKG |
| | | RFTISRDNARNTLYLQMSNLKPEDTAVYYCADRDLYCSGSMCKDVLGGARYDFRGQGTQVTVSS |
| 205 | 2LCRV4 | QVQLQESGGGLVEPGGSLRLSCAASGFRFSSYAMSWVRQAPGKGLERVSAINSDGDKTSYADSVKG |
| | | RFTISRDNARNTLYLQMSNLKPEDTAVYYCADRDLYCSGSMCKDVLGGARYDFRGQGTQVTVSS |
| 206 | 2LCRV3 | QVQLQESGGGMVEPGGSLRLSCAASGFRFSSYAMSWVRQAPGKGLERVSAINSDGDKTSYADSVKG |
| | | RFTISRDNARNTLYLQMNNLKPEDTAVYYCADRDLYCSGSMCKDVLGGARYDFRGQGTQVTVSS |
| 207 | 1LCRV52 | QVQLQESGGGLVQSGESLRLSCAASGLRFSSYAMSWVRQAPGKGLERVSAINSDGDKTSYADSVKG |
| | | RFTISRDNARNTLYLQMSNLKPEDTAVYYCADRDLYCSGSMCKDVLGGARYDFRGQGTQVTVSS |
| 208 | 1LCRV4 | QVQLQESGGGLVQPGGSLKLSCAASGFTFNWYTMAWYRQVPGEERKMVATITGASGDTKYADSVK |
| | | GRFTISRDNAKNTVTLQMNSLKPGDAAVYYCHAYLTYDSGSVKGVNYWGQGTQVTVSS |
| 209 | 1LCRV13 | QVQLQESGGGLVRPGGSLKLSCAASGFTFNWYTMAWYRQVPGEERKMVATITGASGDTKYADSVK |
| | | GRFTISRDNAKNTVTLQMNSLKPGDTAVYYCHAYLTYDSGSVKGVNYWGQGTQVTVSS |
| 210 | 2LCRV1 | QVQLQESGGGSVQPGGSLKLSCAASGFTFNWYTMAWYRQVPGEERKMVATITGASGDTKYADSVK |
| | | GRFTISRDNAKNTVTLQMNSLKPGDTAVYYCHAYLTYDSGSVKGVNYWGQGTQVTVSS |
| 211 | 1LCRV81 | QVQLQESGGGSVQPGGSLKLSCAASGFTFNWYTMAWYRQVPGEERKMVATITGASGDTKYADSVK |
| | | GRSTISRDNAKNTVTLQMNSLKPGDTAVYYCHACLTYDSGSVKGVNYWGQGTQVTVSS |
| 212 | 1LCRV27 | QVQLQESGGGFVQPGGSLKLSCAASGFTFNWYTMAWYRQVPGEERKMVATITGASGDTKYADSVK |
| | | GRFTISRDNAKNTVTLQMNSLKPGDTAVYYCHAYLTYDSGSVKGVNYWGQGTQVTVSS |
| 213 | 1LCRV34 | QVQLQESGGGLVQPGGSLKLSCAASGFTFNWYTMAWYRQVPGEERKMVATITGASGDTKYADSVK |
| | | GRFTISRDNAKNTVTLQMNSLKPGDTAVYYCHAYLTYDSGSAKGVNYWGQGTQVTVSS |
| 214 | 1LCRV31 | QVQLQESGGGLVQPGGSLGLSCAASGSLLNIYAMGWYRQAPGRQRELVATVTSSGTAEYADSVKGR |
| | | FTISRDNAKNTVYLQMNSLRPEDTGVYYCNAHLRYGDYVRGPPEYNYWGQGTQVTVSS |
| 215 | 1LCRV28 | QVQLQESGGGLVQPGGSLRLSCAASGGTLGYYAIGWFRQAPGKEREAVSCITSSDTSAYYADSAKGR |
| | | FTISRDNAKNTMYLQMNNLKPEDTAVYYCAAGYYFRDYSDSYYYTGTGMKVWGKGTQVTVSS |
| 216 | 2LCRV11 | QVQLQESGGGLVQPGGSTRLSCAASGFTLDIYAIGWFRQAPGKEHEGVSWIVGNDGRTYYIDSVKGR |
| | | FTISRDNAKNTVYLEMNSLKPEDTAVYYCAAKFWPRYYSGRPPVGRDGYDYWGQGTQVTVSS |
| 217 | 1LCRV47 | QVQLQESGGGLVQPGGSLILSCTISGASLRDRRVTWSRQGPGKSLEIIAVMAPDYGVHYFGSLEGRVA |
| | | VRGDVVKNTVYLQVNALKPEDTAIYWCSMGNIRGLGTQVTVSS |

TABLE 8
Y. pestis LcrV SAb DNA Sequences

| SEQ ID | | |
|---|---|---|
| NO | Name | Sequence |
| 218 | 1LCRV32 | CAGGTGCAGCTGCAGGAGTCTGGGGGAGGCATGGTAGAACCTGGGGGTTCTCTGAGACTCTCCT |
| | | GTGCAGCCTCTGGATTCCGCTTCAGTAGTTATGCTATGAGTTGGGTCCGCCAGGCTCCAGGAAAG |
| | | GGGCTCGAGCGGGTCTCGGCTATTAATAGTGATGGTGATAAAACAAGCTATGCAGACTCCGTGA |
| | | AGGGCCGATTTACCATCTCCAGAGACAACGCCAGGAACACGCTGTATCTGCAAATGAGCAACCT |
| | | GAAACCTGAAGACACGGCCGTGTATTACTGTGCAGACCGAGATTTGTACTGTTCAGGCTCTATGT |
| | | GTAAGGACGTCTTGGGGGGAGCACGCTATGACTTTCGGGGCCAGGGGACCCAGGTCACCGTCTC |
| | | CAGCGGCCGCTACCCGTACGACGTTCCGGACTACGGTTCCGGCCGAGCATAG |
| 219 | 2LCRV4 | CAGGTGCAGCTGCAGGAGTCTGGAGGAGGCTTGGTAGAACCTGGGGGTTCTCTGAGACTCTCCT |
| | | GTGCAGCCTCTGGATTCCGCTTCAGTAGTTATGCTATGAGTTGGGTCCGCCAGGCTCCAGGAAAG |
| | | GGGCTCGAGCGGGTCTCAGCTATTAATAGTGATGGTGATAAAACAAGCTATGCAGACTCCGTGA |
| | | AGGGCCGATTTACCATCTCCAGAGACAACGCCAGGAACACGCTGTATCTGCAAATGAGCAACCT |
| | | GAAACCTGAAGACACGGCCGTGTATTACTGTGCAGACCGAGATTTGTACTGTTCAGGCTCTATGT |
| | | GTAAGGACGTCTTGGGGGGAGCACGCTATGACTTTCGGGGCCAGGGGACCCAGGTCACCGTCTC |
| | | CAGCGGCCGCTACCCGTACGACGTTCCGGACTACGGTTCCGGCCGAGCATAG |
| 220 | 2LCRV3 | CAGGTGCAGCTGCAGGAGTCTGGGGGAGGCATGGTAGAACCTGGGGGTTCTCTGAGACTCTCTTGT |
| | | GCAGCCTCTGGATTCCGCTTCAGTAGTTATGCTATGAGTTGGGTCCGCCAGGCTCCAGGAAAGGGG |
| | | CTCGAGCGGGTCTCGGCTATTAATAGTGATGGTGATAAAACAAGCTATGCAGACTCCGTGAAGGGC |
| | | CGATTCACCATCTCCAGAGACAACGCCAGGAACACGCTGTATCTGCAAATGAACAACCTGAAACCT |
| | | GAAGACACGGCCGTGTATTACTGTGCAGACCGAGATTTGTACTGTTCGGGCTCTATGTGTAAGGAC |
| | | GTCTTGGGGGGAGCACGCTATGACTTTCGGGGCCAGGGGACCCAGGTCACCGTCTCCAGCGGCCGC |
| | | TACCCGTACGACGTTCCGGACTACGGTTCCGGCCGAGCATAG |
| 221 | 1LCRV52 | CAGGTGCAGCTGCAGGAGTCTGGGGGAGGCTTGGTGCAGTCTGGCGAGTCTCTCAGACTCTCCTG |
| | | TGCAGCCTCTGGACTCCGCTTCAGTAGTTATGCTATGAGTTGGGTCCGCCAGGCTCCAGGAAAGG |
| | | GGCTCGAGCGGGTCTCGGCTATTAATAGTGATGGTGATAAAACAAGCTATGCAGACTCCGTGAA |
| | | GGGCCGATTTACCATCTCCAGAGACAACGCCAGGAACACGCTGTATCTGCAAATGAGCAACCTG |
| | | AAACCTGAAGACACGGCCGTGTATTACTGTGCAGACCGAGATTTGTACTGTTCAGGCTCTATGTG |
| | | TAAGGACGTCTTGGGGGGAGCACGCTATGACTTTCGGGGCCAGGGGACCCAGGTCACCGTCTCC |
| | | AGCGGCCGCTACCCGTACGACGTTCCGGACTACGGTTCCGGCCGAGCATAG |
| 222 | 1LCRV4 | CAGGTGCAGCTGCAGGAGTCTGGAGGAGGCCTGGTGCAGCCTGGGGGGTCTCTGAAACTCTCCT |
| | | GTGCAGCCTCTGGATTCACCTTCAATTGGTATACCATGGCCTGGTATCGCCAGGTTCCAGGGGAG |
| | | GAGCGCAAAATGGTCGCCACAATTACAGGTGCTAGTGGTGACACAAAATATGCAGACTCCGTGA |
| | | AGGGCCGGTTCACCATCTCCAGAGACAATGCCAAGAACACGGTGACACTGCAAATGAACAGCCT |
| | | TAAACCTGGAGACGCGGCCGTCTATTACTGTCATGCCTACCTAACCTACGACTCGGGGTCCGTCA |
| | | AAGGAGTTAACTACTGGGGCCAGGGGACCCAGGTCACCGTCTCCAGCGGCCGCTACCCGTACGA |
| | | CGTTCCGGACTACGGTTCCGGCCGAGCATAG |
| 223 | 1LCRV13 | CAGGTGCAGCTGCAGGAGTCTGGGGGAGGCTTGGTGCGGCCTGGGGGGTCTCTGAAACTCTCCT |
| | | GTGCAGCCTCTGGATTCACCTTCAATTGGTATACCATGGCCTGGTATCGCCAGGTTCCAGGGGAG |
| | | GAGCGCAAAATGGTCGCCACAATTACAGGTGCTAGTGGTGACACAAAATATGCAGACTCCGTGA |
| | | AGGGCCGGTTCACCATCTCCAGAGACAATGCCAAGAACACGGTGACACTGCAAATGAACAGCCT |
| | | TAAACCTGGAGACACGGCCGTCTATTACTGTCATGCCTACCTAACCTACGACTCGGGGTCCGTCA |
| | | AAGGAGTTAACTACTGGGGCCAGGGGACCCAGGTCACCGTCTCCAGCGGCCGCTACCCGTACGA |
| | | CGTTCCGGACTACGGTTCCGGCCGAGCATAG |
| 224 | 2LCRV1 | CAGGTGCAGCTGCAGGAGTCTGGGGGAGGCTCGGTGCAGCCTGGGGGGTCTCTGAAACTCTCCT |
| | | GTGCAGCCTCTGGATTCACCTTCAATTGGTATACCATGGCCTGGTATCGCCAGGTTCCAGGGGAG |
| | | GAGCGCAAAATGGTTGCCACAATTACAGGTGCTAGTGGTGACACAAAATATGCAGACTCCGTGA |
| | | AGGGCCGGTTCACCATCTCCAGAGACAATGCCAAGAACACGGTGACACTGCAAATGAACAGCCT |
| | | TAAACCTGGAGACACGGCCGTCTATTACTGTCATGCCTACCTAACCTACGACTCGGGGTCCGTCA |
| | | AAGGAGTTAACTACTGGGGCCAGGGGACCCAGGTCACCGTCTCCAGCGGCCGCTACCCGTACGA |
| | | CGTTCCGGACTACGGTTCCGGCCGAGCATAG |
| 225 | 1LCRV81 | CAGGTGCAGCTGCAGGAGTCTGGGGGAGGCTCGGTGCAGCCTGGGGGGTCTCTGAAACTCTCCT |
| | | GTGCAGCCTCTGGATTCACCTTCAATTGGTATACCATGGCCTGGTATCGCCAGGTTCCAGGGGAG |
| | | GAGCGCAAAATGGTCGCCACAATTACAGGTGCTAGTGGTGACACAAAATATGCAGACTCCGTGA |
| | | AGGGCCGGTCCACCATCTCCAGAGACAATGCCAAGAACACGGTGACACTGCAAATGAACAGCCT |
| | | TAAACCTGGAGACACGGCCGTCTATTACTGTCATGCCTGCCTAACCTACGACTCGGGGTCCGTCA |
| | | AAGGAGTTAACTACTGGGGTCAGGGGACCCAGGTCACCGTCTCCAGCGGCCGCTACCCGTACGA |
| | | CGTTCCGGACTACGGTTCCGGCCGAGCATAG |
| 226 | 1LCRV27 | CAGGTGCAGCTGCAGGAGTCTGGGGGAGGCTTCGTGCAGCCTGGGGGGTCTCTGAAACTCTCCT |
| | | GTGCAGCCTCTGGATTCACCTTCAATTGGTATACCATGGCCTGGTATCGCCAGGTTCCAGGGGAG |
| | | GAGCGCAAAATGGTCGCCACAATTACAGGTGCTAGTGGTGACACAAAATATGCAGACTCCGTGA |
| | | AGGGCCGGTTCACCATCTCCAGAGACAATGCCAAGAACACGGTGACACTGCAAATGAACAGCCT |
| | | TAAACCTGGAGACACGGCCGTCTATTACTGTCATGCCTACCTAACCTACGACTCGGGGTCCGTCA |
| | | AAGGAGTTAACTACTGGGGCCAGGGGACCCAGGTCACCGTCTCCAGCGGCCGCTACCCGTACGA |
| | | CGTTCCGGACTACGGTTCCGGCCGAGCATAG |
| 227 | 1LCRV34 | CAGGTGCAGCTGCAGGAGTCTGGGGGAGGCCTGGTGCAGCCTGGGGGGTCTCTGAAACTCTCCT |
| | | GTGCAGCCTCTGGATTCACCTTCAATTGGTATACCATGGCCTGGTATCGCCAGGTTCCAGGGGAG |
| | | GAGCGCAAAATGGTCGCCACAATTACAGGTGCTAGTGGTGACACAAAATATGCAGACTCCGTGA |
| | | AGGGCCGGTTCACCATCTCCAGAGACAATGCCAAGAACACGGTGACACTGCAAATGAACAGCCT |
| | | TAAACCTGGAGACACGGCCGTCTATTACTGTCATGCCTACCTAACCTACGACTCGGGGTCCGCCA |
| | | AAGGAGTTAACTACTGGGGCCAGGGGACCCAGGTCACCGTCTCCAGCGGCCGCTACCCGTACGA |
| | | CGTTCCGGACTACGGTTCCGGCCGAGCATAG |
| 228 | 1LCRV31 | CAGGTGCAGCTGCAGGAGTCTGGGGGAGGCTTGGTGCAGCCTGGGGGGTCTCTAGGACTCTCCT |
| | | GTGCAGCCTCTGGAAGCCTCTTAAATATCTATGCCATGGGCTGGTACCGCCAGGCTCCAGGGAGA |
| | | CAGCGCGAGTTGGTCGCAACTGTAACGAGTAGTGGAACCGCAGAATATGCAGACTCCGTGAAGG |
| | | GCCGATTCACCATCTCTAGAGACAACGCCAAGAACACGGTGTATCTGCAAATGAACAGCCTGAG |
| | | ACCTGAGGACACGGGCGTCTATTACTGTAATGCACATCTCAGATATGGCGACTATGTCCGTGGCC |
| | | CTCCGGAGTATAACTACTGGGGCCAGGGGACCCAGGTCACCGTCTCCAGCGGCCGCTACCCGTA |
| | | CGACGTTCCGGACTACGGTTCCGGCCGAGCATAG |
| 229 | 1LCRV28 | CAGGTGCAGCTGCAGGAGTCTGGGGGAGGCTTGGTGCAGCCTGGGGGGTCTCTGAGACTCTCCT |
| | | GTGCAGCCTCTGGAGGCACTTTGGGTTACTATGCCATAGGCTGGTTCCGCCAGGCCCCAGGGAAG |
| | | GAGCGCGAGGCGGTCTCCTGTATTACTAGTAGTGACACTAGCGCATACTATGCAGACTCCGCGA |
| | | AGGGCCGATTCACCATCTCCAGAGACAACGCCAAGAACACGATGTATCTGCAAATGAACAACCT |
| | | GAAACCTGAGGACACAGCCGTTTATTACTGTGCAGCCGGTTACTATTTTAGAGACTATAGTGACA |
| | | GTTACTACTACACGGGGACGGGTATGAAAGTCTGGGGCAAAGGGACCCAGGTCACCGTCTCCAG |
| | | CGGCCGCTACCCGTACGACGTTCCGGACTACGGTTCCGGCCGAGCATAG |
| 230 | 2LCRV11 | CAGGTGCAGCTGCAGGAGTCTGGGGGAGGCTTGGTGCAGCCTGGGGGGTCTACGAGACTCTCCT |
| | | GTGCAGCCTCTGGATTCACTTTGGATATTTATGCTATAGGCTGGTTCCGCCAGGCCCCAGGGAAG |
| | | GAGCATGAGGGGGTCTCGTGGATTGTTGGTAATGATGGTAGGACATACTACATAGACTCCGTGA |
| | | AGGGCCGATTCACCATCTCCAGAGACAACGCCAAGAACACGGTGTATCTTGAAATGAACAGCCT |
| | | GAAACCTGAGGATACAGCCGTTTATTACTGCGCAGCTAAGTTCTGGCCCCGATATTATAGTGGTA |
| | | GGCCTCCAGTAGGGAGGGATGGCTATGACTATTGGGGCCAGGGGACCCAGGTCACCGTCTCCAG |
| | | CGGCCGCTACCCGTACGACGTTCCGGACTACGGTTCCGGCCGAGCATAG |
| 231 | 1LCRV47 | CAGGTGCAGCTGCAGGAGTCTGGGGGAGGCTTGGTGCAGCCTGGCGGGTCTCTGATACTCTCCTG |
| | | TACAATCTCGGGAGCCTCGCTCCGAGACCGACGCGTCACCTGGAGTCGCCAAGGTCCAGGGAAA |
| | | TCGCTTGAGATCATCGCAGTTATGGCGCCGGATTACGGGGTCCATTACTTTGGCTCCCTGGAGGG |
| | | GCGAGTTGCCGTCCGAGGAGACGTCGTCAAGAATACAGTATATCTCCAAGTAAACGCCCTGAAA |
| | | CCCGAAGACACAGCCATCTATTGGTGCAGTATGGGGAATATCCGGGGCCTGGGGACCCAGGTCA |
| | | CCGTCTCCAGCGGCCGCTACCCGTACGACGTTCCGGACTACGGTTCCGGCCGAGCATAG |

TABLE 9
F1 SAb Groups

| Group | Name | SEQ ID NO |
|---|---|---|
| 1 | 3F55, 3F85 | 168, 169 |
| 2 | 3F44 | 170 |
| 3 | 3F31, 3F61, 4F1, 4F6, 4F34 | 171-175 |
| 4 | 4F27 | 176 |
| 5 | 3F26 | 177 |
| 6 | 4F59 | 178 |
| 7 | 3F5, 4F57 | 179-180 |
| 8 | 4F75 | 181 |
| 9 | 3F59, 4F78 | 182-183 |
| 10 | 3F1, 3F65 | 184-185 |

TABLE 10
LcrV SAb Groups

| Group | Name | SEQ ID NO |
|---|---|---|
| 1 | 1LCRV32, 2LCRV4, 2LCRV3, 1LCRV52 | 204-207 |
| 2 | 1CLRV4, 1LCRV13, 2LCRV1, 1LCRV81, | 208-213 |
| | 1LCRV27, 1LCRV34 | |
| 3 | 1LCRV31 | 214 |
| 4 | 1LCRV28 | 215 |
| 5 | 2LCRV11 | 216 |
| 6 | 1LCRV47 | 217 |

TABLE 11
Binding Kinetics of LcrV and F1 Sabs

| | | BIACORE K_{D} | Microcal K_{D} |
|---|---|---|---|
| Name | SEQ ID NO | (nM) | (nM) |
| 1LCRV13 | 209 | 0.00063 | 3.2 |
| 1LCRV28 | 215 | 0.19 | 0.20 |
| 1LCRV31 | 214 | 0.0019 | 0.76 |
| 1LCRV32 | 204 | 22 | 26 |
| 1LCRV47 | 217 | >1000 | no heat |
| 1LCRV81 | 211 | 3.5 | Error |
| 2LCRV11 | 216 | 8.2 | Error |
| 3F1 | 184 | 97 | 110 |
| 3F5 | 179 | 47 | 83 |
| 3F26 | 177 | — | — |
| 3F44 | 170 | — | — |
| 3F55 | 168 | 2.2 | 190 |
| 3F59 | 182 | 5.9 | no heat |
| 3F61 | 175 | 68 | 290 |
| 3F85 | 169 | 15 | 110 |
| 4F1 | 173 | 520 | error |
| 4F6 | 172 | 34 | 80 |
| 4F27 | 176 | — | — |
| 4F34 | 171 | 390 | error |
| 4F59 | 178 | 27 | 83 |
| 4F75 | 181 | 6/9 | error |
| 4F78 | 183 | 6/28 | error |

TABLE 12
Binding Constants of LcrV SAbs

| Name | SEQ ID NO | kₐ (M⁻¹ s⁻¹) | k_{d} (s⁻¹) | K_{D} (nM) |
|---|---|---|---|---|
| 1LCRV13 | 209 | 2.5 × 10⁵ | 1.6 × 10⁻⁶ | 0.00063 |
| 1LCRV28 | 215 | 4.3 × 10⁵ | 8.1 × 10⁻⁵ | 0.19 |
| 1LCRV31 | 214 | 1.7 × 10⁵ | 3.1 × 10⁻⁷ | 0.0019 |
| 1LCRV32 | 204 | 3.4 × 10⁵ | 7.3 × 10⁻³ | 22 |
| 1LCRV47 | 217 | n.b. | n.b. | — |
| 1LCRV81 | 211 | 1.8 × 10⁵ | 6.3 × 10⁻⁴ | 3.5 |
| 2LCRV11 | 216 | 8.8 × 10⁵ | 7.2 × 10⁻³ | 8.2 |

Although specific embodiments have been described in detail in the foregoing description and illustrated in the drawings, various other embodiments, changes, and modifications to the disclosed embodiment(s) will become apparent to those skilled in the art. All such other embodiments, changes, and modifications are intended to come within the spirit and scope of the appended claims.

What is claimed is:

1. A single-domain antibody against Yersinia pestis (Y. pestis) SAb protein comprising:
a first framing region (“FR”) sequence comprising SEQ ID No:79;
a first complementarity determining region (“CDR”) sequence comprising SEQ ID No:1;
a second FR sequence comprising SEQ ID No:103, the first CDR sequence being positioned between the first FR sequence and the second FR sequence;
a second CDR sequence comprising SEQ ID No:27;
a third FR sequence comprising SEQ ID No: 121, the second CDR sequence being positioned between the second FR sequence and the third FR sequence;
a third CDR sequence comprising SEQ ID No.54; and
a fourth FR sequence comprising SEQ ID No:147, the third CDR sequence being positioned between the third FR sequence and the fourth FR sequence.

2. The single-domain antibody of claim 1, wherein the at least one single-domain antibody further comprises:
at least one of a protein tag, a protein domain tag, or a chemical tag.

3. The single-domain antibody of claim 1, further comprising:
a plurality of single-domain antibodies, wherein the single-domain antibodies of the plurality are against the Y. pestis F1 protein and a Y. pestis YscF protein or a Y. pestis LcrV protein.

4. A polypeptide comprising:
a plurality of the single-domain antibodies of claim 1 organized into a chain.

5. The polypeptide of claim 4, wherein at least a portion of the plurality of single-domain antibodies comprising the polypeptide is against a different epitope on the F1 protein.

6. The polypeptide of claim 4, wherein the plurality of single-domain antibodies comprising the polypeptide is against the Y. pestis F1 protein and a Y. pestis YscF protein or a Y. pestis LcrV protein.

7. The polypeptide of claim 4, further comprising:
a fusion protein.

8. The polypeptide of claim 4, further comprising:
a multivalent protein complex such that the single-domain antibodies of the plurality are joined together with at least one linker molecule.

9. The polypeptide of claim 4, wherein at least one of the plurality of single-domain antibodies comprising the polypeptide further comprises:
at least one of a protein tag, a protein domain tag, or a chemical tag.

10. At least one isolated nucleotide sequence encoding the at least one single-domain antibody of claim 1, wherein the isolated nucleotide sequence is selected from the group consisting of SEQ ID No:154.


SEQUENCE LISTING (231 sequences)

SEQ ID NO: 1 - protein, 10 aa, synthetic construct
  REGION 1..10: note = Generated by phage peptide display
GRTWRAYYMG

SEQ ID NO: 2 - protein, 10 aa, synthetic construct
  REGION 1..10: note = Generated by phage peptide display
GRAFSNYAMA

SEQ ID NO: 3 - protein, 10 aa, synthetic construct
  REGION 1..10: note = Generated by phage peptide display
GRTFSRYAMG

SEQ ID NO: 4 - protein, 10 aa, synthetic construct
  REGION 1..10: note = Generated by phage peptide display
QRTFSRYSLG

SEQ ID NO: 5 - protein, 10 aa, synthetic construct
  REGION 1..10: note = Generated by phage peptide display
GRTFSSHAMA

SEQ ID NO: 6 - protein, 13 aa, synthetic construct
  REGION 1..13: note = Generated by phage peptide display
GRTFGRPFRYTMG

SEQ ID NO: 7 - protein, 10 aa, synthetic construct
  REGION 1..10: note = Generated by phage peptide display
GETVDDLAIG

SEQ ID NO: 8 - protein, 10 aa, synthetic construct
  REGION 1..10: note = Generated by phage peptide display
GMMYIREAIR

SEQ ID NO: 9 - protein, 10 aa, synthetic construct
  REGION 1..10: note = Generated by phage peptide display
GMMYIRYTMR

SEQ ID NO: 10 - protein, 10 aa, synthetic construct
  REGION 1..10: note = Generated by phage peptide display
GRAVNRYHMH

SEQ ID NO: 11 - protein, 10 aa, synthetic construct
  REGION 1..10: note = Generated by phage peptide display
GIIFSDYALT

SEQ ID NO: 12 - protein, 10 aa, synthetic construct
  REGION 1..10: note = Generated by phage peptide display
ARIFSIYAMV

SEQ ID NO: 13 - protein, 10 aa, synthetic construct
  REGION 1..10: note = Generated by phage peptide display
GVIASISVLR

SEQ ID NO: 14 - protein, 10 aa, synthetic construct
  REGION 1..10: note = Generated by phage peptide display
GTTFRSLVMK

SEQ ID NO: 15 - protein, 10 aa, synthetic construct
  REGION 1..10: note = Generated by phage peptide display
GFTFSNYAMS

SEQ ID NO: 16 - protein, 10 aa, synthetic construct
  REGION 1..10: note = Generated by phage peptide display
GFTFRNYAMS

SEQ ID NO: 17 - protein, 10 aa, synthetic construct
  REGION 1..10: note = Generated by phage peptide display
GFTFSSYAMS

SEQ ID NO: 18 - protein, 10 aa, synthetic construct
  REGION 1..10: note = Generated by phage peptide display
GFTFSTSAMS

SEQ ID NO: 19 - protein, 10 aa, synthetic construct
  REGION 1..10: note = Generated by phage peptide display
GFTFSTNAMS

SEQ ID NO: 20 - protein, 10 aa, synthetic construct
  REGION 1..10: note = Generated by phage peptide display
GFRFSSYAMS

SEQ ID NO: 21 - protein, 10 aa, synthetic construct
  REGION 1..10: note = Generated by phage peptide display
GLRFSSYAMS

SEQ ID NO: 22 - protein, 10 aa, synthetic construct
  REGION 1..10: note = Generated by phage peptide display
GFTFNWYTMA

SEQ ID NO: 23 - protein, 10 aa, synthetic construct
  REGION 1..10: note = Generated by phage peptide display
GSLLNIYAMG

SEQ ID NO: 24 - protein, 10 aa, synthetic construct
  REGION 1..10: note = Generated by phage peptide display
GGTLGYYAIG

SEQ ID NO: 25 - protein, 10 aa, synthetic construct
  REGION 1..10: note = Generated by phage peptide display
GFTLDIYAIG

SEQ ID NO: 26 - protein, 10 aa, synthetic construct
  REGION 1..10: note = Generated by phage peptide display
GASLRDRRVT

SEQ ID NO: 27 - protein, 17 aa, synthetic construct
  REGION 1..17: note = Generated by phage peptide display
VMSRSGGTTSYADSVKG

SEQ ID NO: 28 - protein, 17 aa, synthetic construct
  REGION 1..17: note = Generated by phage peptide display
ANWRSGGLTDYADSVKG

SEQ ID NO: 29 - protein, 17 aa, synthetic construct
  REGION 1..17: note = Generated by phage peptide display
AISWSGSSTYYADSVKG

SEQ ID NO: 30 - protein, 17 aa, synthetic construct
  REGION 1..17: note = Generated by phage peptide display
ATTWSGISSDYADSVKG

SEQ ID NO: 31 - protein, 17 aa, synthetic construct
  REGION 1..17: note = Generated by phage peptide display
AIRWNGDNIHYSDSAKG

SEQ ID NO: 32 - protein, 17 aa, synthetic construct
  REGION 1..17: note = Generated by phage peptide display
GITRSGNNIYYSDSVKG

SEQ ID NO: 33 - protein, 17 aa, synthetic construct
  REGION 1..17: note = Generated by phage peptide display
CISGSDGSTYYADSLSG

SEQ ID NO: 34 - protein, 16 aa, synthetic construct
  REGION 1..16: note = Generated by phage peptide display
FVSSTGNPRYTDSVKG

SEQ ID NO: 35 - protein, 16 aa, synthetic construct
  REGION 1..16: note = Generated by phage peptide display
VVSSTGNPHYADSVKG

SEQ ID NO: 36 - protein, 16 aa, synthetic construct
  REGION 1..16: note = Generated by phage peptide display
FISVGGTTNYAGSVKG

SEQ ID NO: 37 - protein, 16 aa, synthetic construct
  REGION 1..16: note = Generated by phage peptide display
QITRSQNINYTGSVKG

SEQ ID NO: 38 - protein, 16 aa, synthetic construct
  REGION 1..16: note = Generated by phage peptide display
QITRRQNINYTGSVKG

SEQ ID NO: 39 - protein, 16 aa, synthetic construct
  REGION 1..16: note = Generated by phage peptide display
AITTGGTTNYADSVKG

SEQ ID NO: 40 - protein, 16 aa, synthetic construct
  REGION 1..16: note = Generated by phage peptide display
IITSGGNTRYADSVKG

SEQ ID NO: 41 - protein, 17 aa, synthetic construct
  REGION 1..17: note = Generated by phage peptide display
FISSPGDRTRYTEAVKG

SEQ ID NO: 42 - protein, 17 aa, synthetic construct
  REGION 1..17: note = Generated by phage peptide display
TINSGGGSTSYAYSVKG

SEQ ID NO: 43 - protein, 17 aa, synthetic construct
  REGION 1..17: note = Generated by phage peptide display
TINIGGGSTSYADSVKG

SEQ ID NO: 44 - protein, 17 aa, synthetic construct
  REGION 1..17: note = Generated by phage peptide display
TINGGGGITSYADSVKG

SEQ ID NO: 45 - protein, 17 aa, synthetic construct
  REGION 1..17: note = Generated by phage peptide display
TINIAGGITSYADSVKG

SEQ ID NO: 46 - protein, 17 aa, synthetic construct
  REGION 1..17: note = Generated by phage peptide display
TINMGGGTTSYADSVKG

SEQ ID NO: 47 - protein, 17 aa, synthetic construct
  REGION 1..17: note = Generated by phage peptide display
TITSAGGSISYVNSVKG

SEQ ID NO: 48 - protein, 17 aa, synthetic construct
  REGION 1..17: note = Generated by phage peptide display
AINSDGDKTSYADSVKG

SEQ ID NO: 49 - protein, 17 aa, synthetic construct
  REGION 1..17: note = Generated by phage peptide display
TITGASGDTKYADSVKG

SEQ ID NO: 50 - protein, 16 aa, synthetic construct
  REGION 1..16: note = Generated by phage peptide display
TVTSSGTAEYADSVKG

SEQ ID NO: 51 - protein, 17 aa, synthetic construct
  REGION 1..17: note = Generated by phage peptide display
CITSSDTSAYYADSAKG

SEQ ID NO: 52 - protein, 17 aa, synthetic construct
  REGION 1..17: note = Generated by phage peptide display
WIVGNDGRTYYIDSVKG

SEQ ID NO: 53 - protein, 16 aa, synthetic construct
  REGION 1..16: note = Generated by phage peptide display
VMAPDYGVHYFGSLEG

SEQ ID NO: 54 - protein, 14 aa, synthetic construct
  REGION 1..14: note = Generated by phage peptide display
GGGMYGPDLYGMTY

SEQ ID NO: 55 - protein, 17 aa, synthetic construct
  REGION 1..17: note = Generated by phage peptide display
GGGSRWYGRTTASWYDY

SEQ ID NO: 56 - protein, 11 aa, synthetic construct
  REGION 1..11: note = Generated by phage peptide display
PAYGLRPPYNY

SEQ ID NO: 57 - protein, 17 aa, synthetic construct
  REGION 1..17: note = Generated by phage peptide display
GRSSWFAPWLTPYEYDY

SEQ ID NO: 58 - protein, 5 aa, synthetic construct
  REGION 1..5: note = Generated by phage peptide display
GVYDY

SEQ ID NO: 59 - protein, 7 aa, synthetic construct
  REGION 1..7: note = Generated by phage peptide display
DWGWRNY

SEQ ID NO: 60 - protein, 12 aa, synthetic construct
  REGION 1..12: note = Generated by phage peptide display
EIYDRRWYRNDY

SEQ ID NO: 61 - protein, 7 aa, synthetic construct
  REGION 1..7: note = Generated by phage peptide display
YLGSRDY

SEQ ID NO: 62 - protein, 8 aa, synthetic construct
  REGION 1..8: note = Generated by phage peptide display
YDGRRPPY

SEQ ID NO: 63 - protein, 8 aa, synthetic construct
  REGION 1..8: note = Generated by phage peptide display
YDGRRRTY

SEQ ID NO: 64 - protein, 8 aa, synthetic construct
  REGION 1..8: note = Generated by phage peptide display
YDGRRSPY

SEQ ID NO: 65 - protein, 7 aa, synthetic construct
  REGION 1..7: note = Generated by phage peptide display
LVGAKDY

SEQ ID NO: 66 - protein, 4 aa, synthetic construct
  REGION 1..4: note = Generated by phage peptide display
NGIY

SEQ ID NO: 67 - protein, 6 aa, synthetic construct
  REGION 1..6: note = Generated by phage peptide display
TASHIP

SEQ ID NO: 68 - protein, 8 aa, synthetic construct
  REGION 1..8: note = Generated by phage peptide display
TARDSRDS

SEQ ID NO: 69 - protein, 8 aa, synthetic construct
  REGION 1..8: note = Generated by phage peptide display
TAANWSAQ

SEQ ID NO: 70 - protein, 8 aa, synthetic construct
  REGION 1..8: note = Generated by phage peptide display
TAGNWSAQ

SEQ ID NO: 71 - protein, 6 aa, synthetic construct
  REGION 1..6: note = Generated by phage peptide display
LVNLAQ

SEQ ID NO: 72 - protein, 21 aa, synthetic construct
  REGION 1..21: note = Generated by phage peptide display
RDLYCSGSMCKDVLGGARYDF

SEQ ID NO: 73 - protein, 14 aa, synthetic construct
  REGION 1..14: note = Generated by phage peptide display
YLTYDSGSVKGVNY

SEQ ID NO: 74 - protein, 14 aa, synthetic construct
  REGION 1..14: note = Generated by phage peptide display
CLTYDSGSVKGVNY

SEQ ID NO: 75 - protein, 14 aa, synthetic construct
  REGION 1..14: note = Generated by phage peptide display
YLTYDSGSAKGVNY

SEQ ID NO: 76 - protein, 16 aa, synthetic construct
  REGION 1..16: note = Generated by phage peptide display
HLRYGDYVRGPPEYNY

SEQ ID NO: 77 - protein, 20 aa, synthetic construct
  REGION 1..20: note = Generated by phage peptide display
GYYFRDYSDSYYYTGTGMKV

SEQ ID NO: 78 - protein, 20 aa, synthetic construct
  REGION 1..20: note = Generated by phage peptide display
KFWPRYYSGRPPVGRDGYDY

SEQ ID NO: 79 - protein, 25 aa, synthetic construct
  REGION 1..25: note = Generated by phage peptide display
QVQLQESGGGLVQAGGSLRLSCAAS

SEQ ID NO: 80 - protein, 25 aa, synthetic construct
  REGION 1..25: note = Generated by phage peptide display
QVQLQESGGGLVQAGGSLRLSCVAS

SEQ ID NO: 81 - protein, 25 aa, synthetic construct
  REGION 1..25: note = Generated by phage peptide display
QVQLQESGGGLVQAGGSLRLSCAVS

SEQ ID NO: 82 - protein, 25 aa, synthetic construct
  REGION 1..25: note = Generated by phage peptide display
QVQLQESGGGLVQAGGSLKLSCTAS

SEQ ID NO: 83 - protein, 25 aa, synthetic construct
  REGION 1..25: note = Generated by phage peptide display
QVQLQESGGGLVQAGDSRILSCTAS

SEQ ID NO: 84 - protein, 25 aa, synthetic construct
  REGION 1..25: note = Generated by phage peptide display
QVQLQESGGGLVQAGGSLRLACAAS

SEQ ID NO: 85 - protein, 25 aa, synthetic construct
  REGION 1..25: note = Generated by phage peptide display
QVQLQESGGGLVQPGGSLRLSCAVS

SEQ ID NO: 86 - protein, 25 aa, synthetic construct
  REGION 1..25: note = Generated by phage peptide display
QVQLQESGGGLVRPGGSLRLSCAVS

SEQ ID NO: 87 - protein, 25 aa, synthetic construct
  REGION 1..25: note = Generated by phage peptide display
QVQLQESGGGSVQPGGSLSLSCSAS

SEQ ID NO: 88 - protein, 25 aa, synthetic construct
  REGION 1..25: note = Generated by phage peptide display
QVQLQESGGGLVQPGGSLSLSCSAS

SEQ ID NO: 89 - protein, 25 aa, synthetic construct
  REGION 1..25: note = Generated by phage peptide display
QVQLQESGGGLVQPGGSLRLSCSAS

SEQ ID NO: 90 - protein, 25 aa, synthetic construct
  REGION 1..25: note = Generated by phage peptide display
QVQLQESGGGLVQPGGSLRLSCAAS

SEQ ID NO: 91 - protein, 25 aa, synthetic construct
  REGION 1..25: note = Generated by phage peptide display
QVQLQESGGGLVRPGGSLRLSCEAS

SEQ ID NO: 92 - protein, 25 aa, synthetic construct
  REGION 1..25: note = Generated by phage peptide display
QVQLQESGGGLVQSGDSLRLSCAAS

SEQ ID NO: 93 - protein, 25 aa, synthetic construct
  REGION 1..25: note = Generated by phage peptide display
QVQLQESGGGMVEPGGSLRLSCAAS

SEQ ID NO: 94 - protein, 25 aa, synthetic construct
  REGION 1..25: note = Generated by phage peptide display
QVQLQESGGGLVEPGGSLRLSCAAS

SEQ ID NO: 95 - protein, 25 aa, synthetic construct
  REGION 1..25: note = Generated by phage peptide display
QVQLQESGGGLVQSGESLRLSCAAS

SEQ ID NO: 96 - protein, 25 aa, synthetic construct
  REGION 1..25: note = Generated by phage peptide display
QVQLQESGGGLVQPGGSLKLSCAAS

SEQ ID NO: 97 - protein, 25 aa, synthetic construct
  REGION 1..25: note = Generated by phage peptide display
QVQLQESGGGLVRPGGSLKLSCAAS

SEQ ID NO: 98 - protein, 25 aa, synthetic construct
  REGION 1..25: note = Generated by phage peptide display
QVQLQESGGGSVQPGGSLKLSCAAS

SEQ ID NO: 99 - protein, 25 aa, synthetic construct
  REGION 1..25: note = Generated by phage peptide display
QVQLQESGGGFVQPGGSLKLSCAAS

SEQ ID NO: 100 - protein, 25 aa, synthetic construct
  REGION 1..25: note = Generated by phage peptide display
QVQLQESGGGLVQPGGSLGLSCAAS

SEQ ID NO: 101 - protein, 25 aa, synthetic construct
  REGION 1..25: note = Generated by phage peptide display
QVQLQESGGGLVQPGGSTRLSCAAS

SEQ ID NO: 102 - protein, 25 aa, synthetic construct
  REGION 1..25: note = Generated by phage peptide display
QVQLQESGGGLVQPGGSLILSCTIS

SEQ ID NO: 103 - protein, 14 aa, synthetic construct
  REGION 1..14: note = Generated by phage peptide display
WFRQAPGKEREFVA

SEQ ID NO: 104 - protein, 14 aa, synthetic construct
  REGION 1..14: note = Generated by phage peptide display
WFRQAPGEERVFVA

SEQ ID NO: 105 - protein, 14 aa, synthetic construct
  REGION 1..14: note = Generated by phage peptide display
WFRQGPGEERQFLA

SEQ ID NO: 106 - protein, 14 aa, synthetic construct
  REGION 1..14: note = Generated by phage peptide display
WFRRAPGKEREFVG

SEQ ID NO: 107 - protein, 14 aa, synthetic construct
  REGION 1..14: note = Generated by phage peptide display
WFRQAPGKEREEIS

SEQ ID NO: 108 - protein, 14 aa, synthetic construct
  REGION 1..14: note = Generated by phage peptide display
WYRQAPGKQREWVA

SEQ ID NO: 109 - protein, 14 aa, synthetic construct
  REGION 1..14: note = Generated by phage peptide display
WYRQAPGKQREWVT

SEQ ID NO: 110 - protein, 14 aa, synthetic construct
  REGION 1..14: note = Generated by phage peptide display
WYRQTPGKTRDWVA

SEQ ID NO: 111 - protein, 14 aa, synthetic construct
  REGION 1..14: note = Generated by phage peptide display
WYRQAPGKEREWVA

SEQ ID NO: 112 - protein, 14 aa, synthetic construct
  REGION 1..14: note = Generated by phage peptide display
WVRQAPGKGLEWVS

SEQ ID NO: 113 - protein, 14 aa, synthetic construct
  REGION 1..14: note = Generated by phage peptide display
WVRLAPGKGLEWVS

SEQ ID NO: 114 - protein, 14 aa, synthetic construct
  REGION 1..14: note = Generated by phage peptide display
WIRQPPGKAREVVA

SEQ ID NO: 115 - protein, 14 aa, synthetic construct
  REGION 1..14: note = Generated by phage peptide display
WVRQAPGKGLERVS

SEQ ID NO: 116 - protein, 14 aa, synthetic construct
  REGION 1..14: note = Generated by phage peptide display
WYRQVPGEERKMVA

SEQ ID NO: 117 - protein, 14 aa, synthetic construct
  REGION 1..14: note = Generated by phage peptide display
WYRQAPGRQRELVA

SEQ ID NO: 118 - protein, 14 aa, synthetic construct
  REGION 1..14: note = Generated by phage peptide display
WFRQAPGKEREAVS

SEQ ID NO: 119 - protein, 14 aa, synthetic construct
  REGION 1..14: note = Generated by phage peptide display
WFRQAPGKEHEGVS

SEQ ID NO: 120 - protein, 14 aa, synthetic construct
  REGION 1..14: note = Generated by phage peptide display
WSRQGPGKSLEIIA

SEQ ID NO: 121 - protein, 32 aa, synthetic construct
  REGION 1..32: note = Generated by phage peptide display
RFTISRDNAKNTVYLQMNNLAPEDTATYYCKA

SEQ ID NO: 122 - protein, 32 aa, synthetic construct
  REGION 1..32: note = Generated by phage peptide display
RFTISRDDAKNTVYLQMNSLKPEDTAVYYCAA

SEQ ID NO: 123 - protein, 32 aa, synthetic construct
  REGION 1..32: note = Generated by phage peptide display
RFTISRDHAKNVMYLQMNGLKPEDTGVYVCAR

SEQ ID NO: 124 - protein, 32 aa, synthetic construct
  REGION 1..32: note = Generated by phage peptide display
RFTISRDNAKNTGYLQMNNLKPEDTGVYYCAA

SEQ ID NO: 125 - protein, 32 aa, synthetic construct
  REGION 1..32: note = Generated by phage peptide display
RFTISRDLAKNTLYLQMNSLKPEDTAVYYCAR

SEQ ID NO: 126 - protein, 32 aa, synthetic construct
  REGION 1..32: note = Generated by phage peptide display
RFTISRDNAKNTVYLQMNSLKPEDTAVYYCNA

SEQ ID NO: 127 - protein, 32 aa, synthetic construct
  REGION 1..32: note = Generated by phage peptide display
RFTISRDNVKNTVYLQMNSLKLEDTAVYYCYA

SEQ ID NO: 128 - protein, 32 aa, synthetic construct
  REGION 1..32: note = Generated by phage peptide display
RFTISRDNAKNTVYLQMNSLTPEDTAVYYCNT

SEQ ID NO: 129 - protein, 32 aa, synthetic construct
  REGION 1..32: note = Generated by phage peptide display
RFTVSRDNAKNTLYLQMNSLKPEDTAVYYCNS

SEQ ID NO: 130 - protein, 32 aa, synthetic construct
  REGION 1..32: note = Generated by phage peptide display
RFTVSRDNAKNTVHLQMNSLKPEDTAVYYCHA

SEQ ID NO: 131 - protein, 32 aa, synthetic construct
  REGION 1..32: note = Generated by phage peptide display
RFTVSRDNAKNTVHLQMNSLKPEDAAVYYCHA

SEQ ID NO: 132 - protein, 32 aa, synthetic construct
  REGION 1..32: note = Generated by phage peptide display
RFTTSRDNARNTVYLQMNSLKPEDTAVYYCNT

SEQ ID NO: 133 - protein, 32 aa, synthetic construct
  REGION 1..32: note = Generated by phage peptide display
RFTISRDNAKNALYLQMNGLKPEDTAVYYCNA

SEQ ID NO: 134 - protein, 32 aa, synthetic construct
  REGION 1..32: note = Generated by phage peptide display
RFTISRDNAKNTLYLQMNSLKPEDTAVYYCAK

SEQ ID NO: 135 - protein, 32 aa, synthetic construct
  REGION 1..32: note = Generated by phage peptide display
RFTISRDNAKNTMYLQMNSLKPEDTAVYYCAQ

SEQ ID NO: 136 - protein, 32 aa, synthetic construct
  REGION 1..32: note = Generated by phage peptide display
RFTISRHNAKNTLYLQMNSLKPEDTAVYYCAK

SEQ ID NO: 137 - protein, 32 aa, synthetic construct
  REGION 1..32: note = Generated by phage peptide display
RFTISRDNAKNTLYLQMNMLKPEDTAVYYCAR

SEQ ID NO: 138 - protein, 32 aa, synthetic construct
  REGION 1..32: note = Generated by phage peptide display
RFTISRDNARNTLYLQMSNLKPEDTAVYYCAD

SEQ ID NO: 139 - protein, 32 aa, synthetic construct
  REGION 1..32: note = Generated by phage peptide display
RFTISRDNARNTLYLQMNNLKPEDTAVYYCAD

SEQ ID NO: 140 - protein, 32 aa, synthetic construct
  REGION 1..32: note = Generated by phage peptide display
RFTISRDNAKNTVTLQMNSLKPGDAAVYYCHA

SEQ ID NO: 141 - protein, 32 aa, synthetic construct
  REGION 1..32: note = Generated by phage peptide display
RFTISRDNAKNTVTLQMNSLKPGDTAVYYCHA

SEQ ID NO: 142 - protein, 32 aa, synthetic construct
  REGION 1..32: note = Generated by phage peptide display
RSTISRDNAKNTVTLQMNSLKPGDTAVYYCHA

SEQ ID NO: 143 - protein, 32 aa, synthetic construct
  REGION 1..32: note = Generated by phage peptide display
RFTISRDNAKNTVYLQMNSLRPEDTGVYYCNA

SEQ ID NO: 144 - protein, 32 aa, synthetic construct
  REGION 1..32: note = Generated by phage peptide display
RFTISRDNAKNTMYLQMNNLKPEDTAVYYCAA

SEQ ID NO: 145 - protein, 32 aa, synthetic construct
  REGION 1..32: note = Generated by phage peptide display
RFTISRDNAKNTVYLEMNSLKPEDTAVYYCAA

SEQ ID NO: 146 - protein, 32 aa, synthetic construct
  REGION 1..32: note = Generated by phage peptide display
RVAVRGDVVKNTVYLQVNALKPEDTAIYWCSM

SEQ ID NO: 147 - protein, 11 aa, synthetic construct
  REGION 1..11: note = Generated by phage peptide display
WGKGTQVTVSS

SEQ ID NO: 148 - protein, 11 aa, synthetic construct
  REGION 1..11: note = Generated by phage peptide display
WGQGTQVTVSS

SEQ ID NO: 149 - protein, 11 aa, synthetic construct
  REGION 1..11: note = Generated by phage peptide display
RGQGTQVTVSS

SEQ ID NO: 150 - protein, 11 aa, synthetic construct
  REGION 1..11: note = Generated by phage peptide display
WGRGTQVTVSS

SEQ ID NO: 151 - protein, 11 aa, synthetic construct
  REGION 1..11: note = Generated by phage peptide display
LSQGTQVTVSS

SEQ ID NO: 152 - protein, 11 aa, synthetic construct
  REGION 1..11: note = Generated by phage peptide display
TGQGTQVTVSS

SEQ ID NO: 153 - protein, 11 aa, synthetic construct
  REGION 1..11: note = Generated by phage peptide display
RGLGTQVTVSS

SEQ ID NO: 154 - protein, 123 aa, synthetic construct
  REGION 1..123: note = Generated by phage peptide display
QVQLQESGGGLVQAGGSLRLSCAASGRTWRAYYMGWFRQAPGKEREFVAVMSRSGGTTSYADSVKGRFTISRDNAKNTVYLQMNNLAPEDTATYYCKAGGGMYGPDLYGMTYWGKGTQVTVSS

SEQ ID NO: 155 - protein, 126 aa, synthetic construct
  REGION 1..126: note = Generated by phage peptide display
QVQLQESGGGLVQAGGSLRLSCVASGRAFSNYAMAWFRQAPGKEREFVAANWRSGGLTDYADSVKGRFTISRDDAKNTVYLQMNSLKPEDTAVYYCAAGGGSRWYGRTTASWYDYWGQGTQVTVSS

SEQ ID NO: 156 - protein, 120 aa, synthetic construct
  REGION 1..120: note = Generated by phage peptide display
QVQLQESGGGLVQAGGSLRLSCAVSGRTFSRYAMGWFRQAPGKEREFVAAISWSGSSTYYADSVKGRFTISRDHAKNVMYLQMNGLKPEDTGVYVCARPAYGLRPPYNYRGQGTQVTVSS

SEQ ID NO: 157 - protein, 126 aa, synthetic construct
  REGION 1..126: note = Generated by phage peptide display
QVQLQESGGGLVQAGGSLKLSCTASQRTFSRYSLGWFRQAPGEERVFVAATTWSGISSDYADSVKGRFTISRDNAKNTGYLQMNNLKPEDTGVYYCAAGRSSWFAPWLTPYEYDYWGRGTQVTVSS

SEQ ID NO: 158 - protein, 114 aa, synthetic construct
  REGION 1..114: note = Generated by phage peptide display
QVQLQESGGGLVQAGGSLRLSCAASGRTFSSHAMAWFRQGPGEERQFLAAIRWNGDNIHYSDSAKGRFTISRDLAKNTLYLQMNSLKPEDTAVYYCARGVYDYWGQGTQVTVSS

SEQ ID NO: 159 - protein, 119 aa, synthetic construct
  REGION 1..119: note = Generated by phage peptide display
QVQLQESGGGLVQAGDSRILSCTASGRTFGRPFRYTMGWFRRAPGKEREFVGGITRSGNNIYYSDSVKGRFTISRDNAKNTVYLQMNSLKPEDTAVYYCNADWGWRNYWGQGTQVTVSS

SEQ ID NO: 160 - protein, 121 aa, synthetic construct
  REGION 1..121: note = Generated by phage peptide display
QVQLQESGGGLVQAGGSLRLACAASGETVDDLAIGWFRQAPGKEREEISCISGSDGSTYYADSLSGRFTISRDNVKNTVYLQMNSLKLEDTAVYYCYAEIYDRRWYRNDYWGQGTQVTVSS

SEQ ID NO: 161 - DNA, 417 bp, synthetic construct
  misc_feature 1..417: note = Generated by phage peptide display
  source 1..417: mol_type = other DNA
caggtgcagctgcaggagtctgggggaggattggtgcaggctgggggctctctgagactctcctgtgcagcctctggacgcacctggagagcctattacatgggctggttccgccaggctccagggaaggagcgtgagtttgtagcagttatgagtcggagcggtggcaccacatcctatgcggactccgtgaagggccgattcaccatctccagagacaacgccaagaacacggtgtatctacaaatgaacaacctggcacctgaggacacggccacgtattattgtaaggcggggggcggaatgtacgggccggacctgtatggtatgacatactggggcaaagggacccaggtcaccgtctccagcggccgctacccgtacgacgttccggactacggttccggccgagcatag

SEQ ID NO: 162 - DNA, 426 bp, synthetic construct
  misc_feature 1..426: note = Generated by phage peptide display
  source 1..426: mol_type = other DNA
caggtgcagctgcaggagtctggaggaggattggtacaggctgggggctctctgagactctcctgtgtagcctctggacgcgccttcagtaattatgcgatggcctggttccgccaggctccagggaaggagcgtgagtttgtagcagctaattggcggagtggtggtcttacagactatgcagactccgtgaagggccgattcaccatctccagagacgacgccaagaacacggtgtatctgcaaatgaacagcctgaaacctgaggacacggccgtttattactgtgccgccgggggcggtagtcgctggtacgggcgaacaaccgcaagttggtatgactactggggccaggggacccaggtcaccgtctccagcggccgctacccgtacgacgttccggactacggttccggccgagcatag

SEQ ID NO: 163 - DNA, 408 bp, synthetic construct
  misc_feature 1..408: note = Generated by phage peptide display
  source 1..408: mol_type = other DNA
caggtgcagctgcaggagtctgggggaggattggtgcaggctgggggctctctgagactctcctgtgcagtctctggacgcaccttcagtagatatgccatgggctggttccgccaggctccagggaaggagcgtgagtttgtagcagctattagctggagtggtagtagcacatattatgcagactccgtgaagggccgattcaccatctccagagaccacgccaagaacgtgatgtatctgcaaatgaacggcctgaaacctgaggacacgggtgtttatgtctgtgcaagaccagcgtacggactccgccccccgtataattaccggggccaggggacccaggtcaccgtctccagcggccgctacccgtacgacgttccggactacggttccggccgagcatag

SEQ ID NO: 164 - DNA, 426 bp, synthetic construct
  misc_feature 1..426: note = Generated by phage peptide display
  source 1..426: mol_type = other DNA
caggtgcagctgcaggagtctgggggaggattggtgcaggctgggggctctctgaaactctcctgcacagcctctcaacgcaccttcagtcgctatagcttgggctggttccgccaggctccaggtgaggagcgtgtttttgtagccgctactacatggagtggtataagcagtgactatgcagactccgtgaagggccgattcaccatctccagagacaacgccaagaacacggggtatctgcaaatgaacaatttaaaacctgaggacacgggcgtttattactgtgcagcaggacgtagtagctggttcgccccctggttgaccccctatgagtatgattattggggccgggggacccaggtcaccgtctccagcggccgctacccgtacgacgttccggactacggttccggccgagcatag

SEQ ID NO: 165 - DNA, 390 bp, synthetic construct
  misc_feature 1..390: note = Generated by phage peptide display
  source 1..390: mol_type = other DNA
caggtgcagctgcaggagtctgggggaggattggtgcaggctgggggctctctgagactctcctgtgcagcctctggacgcaccttcagtagccatgccatggcctggttccgccagggtccaggagaggagcgtcagtttctagcagctattagatggaatggtgataacatacactattcagactccgcgaagggccgattcaccatctccagagacctcgccaagaacacgctctatctgcaaatgaacagcctgaaacctgaggacacggccgtgtattactgtgcaaggggggtgtatgactactggggccaggggacccaggtcaccgtctccagcggccgctacccgtacgacgttccggactacggttccggccgagcatag

SEQ ID NO: 166 - DNA, 405 bp, synthetic construct
  misc_feature 1..405: note = Generated by phage peptide display
  source 1..405: mol_type = other DNA
caggtgcagctgcaggagtctgggggaggattggtgcaggctggggactctcggatactctcctgtacagcctctggacgcacctttggacgccccttcagatataccatgggctggttccgccgggctccagggaaggagcgtgagtttgtaggaggtattacaagaagtggtaataatatatactattcagactccgtgaagggccgattcaccatctccagagacaacgccaagaacacggtgtatctccaaatgaacagcctgaaacctgaggacacggccgtgtattattgtaacgcagattgggggtggaggaactactggggccaggggacccaggtcaccgtctccagcggccgctacccgtacgacgttccggactacggttccggccgagcatag

SEQ ID NO: 167 - DNA, 411 bp, synthetic construct
  misc_feature 1..411: note = Generated by phage peptide display
  source 1..411: mol_type = other DNA
caggtgcagctgcaggagtctgggggaggcttggtgcaggctggggggtctctgagactcgcctgtgcagcctctggagagactgtcgatgatcttgccatcggctggttccgccaggccccagggaaggagcgtgaggagatttcatgtattagtggtagtgatggtagcacatactatgcagactccctgtcgggccgattcaccatctccagggacaacgtcaagaacacggtgtatctgcaaatgaacagcctgaaacttgaggacacggccgtctattactgttatgcagagatttacgatagacgctggtatcggaacgactactggggccaggggacccaggtcaccgtctccagcggccgctacccgtacgacgttccggactacggttccggccgagcatag

SEQ ID NO: 168 - protein, 115 aa, synthetic construct
  REGION 1..115: note = Generated by phage peptide display
QVQLQESGGGLVQAGGSLRLSCAVSGMMYIREAIRWYRQAPGKQREWVAFVSSTGNPRYTDSVKGRFTISRDNAKNTVYLQMNSLTPEDTAVYYCNTYLGSRDYWGQGTQVTVSS

SEQ ID NO: 169 - protein, 115 aa, synthetic construct
  REGION 1..115: note = Generated by phage peptide display
QVQLQESGGGLVQPGGSLRLSCAVSGMMYIRYTMRWYRQAPGKQREWVAVVSSTGNPHYADSVKGRFTISRDNAKNTVYLQMNSLTPEDTAVYYCNTYLGSRDYWGQGTQVTVSS

SEQ ID NO: 170 - protein, 111 aa, synthetic construct
  REGION 1..111: note = Generated by phage peptide display
QVQLQESGGGLVRPGGSLRLSCAVSGRAVNRYHMHWYRQAPGKQREWVTFISVGGTTNYAGSVKGRFTVSRDNAKNTLYLQMNSLKPEDTAVYYCNSAEYWGQGTQVTVSS

SEQ ID NO: 171 - protein, 116 aa, synthetic construct
  REGION 1..116: note = Generated by phage peptide display
QVQLQESGGGSVQPGGSLSLSCSASGIIFSDYALTWYRQAPGKQREWVAQITRSQNINYTGSVKGRFTVSRDNAKNTVHLQMNSLKPEDTAVYYCHAYDGRRPPYWGQGTQVTVSS

SEQ ID NO: 172 - protein, 116 aa, synthetic construct
  REGION 1..116: note = Generated by phage peptide display
QVQLQESGGGSVQPGGSLSLSCSASGIIFSDYALTWYRQAPGKQREWVAQITRSQNINYTGSVKGRFTVSRDNAKNTVHLQMNSLKPEDTAVYYCHAYDGRRRTYWGQGTQVTVSS

SEQ ID NO: 173 - protein, 116 aa, synthetic construct
  REGION 1..116: note = Generated by phage peptide display
QVQLQESGGGLVQPGGSLSLSCSASGIIFSDYALTWYRQAPGKQREWVAQITRSQNINYTGSVKGRFTVSRDNAKNTVHLQMNSLKPEDTAVYYCHAYDGRRPPYWGQGTQVTVSS

SEQ ID NO: 174 - protein, 116 aa, synthetic construct
  REGION 1..116: note = Generated by phage peptide display
QVQLQESGGGLVQPGGSLSLSCSASGIIFSDYALTWYRQAPGKQREWVAQITRRQNINYTGSVKGRFTVSRDNAKNTVHLQMNSLKPEDTAVYYCHAYDGRRSPYWGQGTQVTVSS

SEQ ID NO: 175 - protein, 116 aa, synthetic construct
  REGION 1..116: note = Generated by phage peptide display
QVQLQESGGGLVQPGGSLRLSCSASGIIFSDYALTWYRQAPGKQREWVAQITRSQNINYTGSVKGRFTVSRDNAKNTVHLQMNSLKPEDAAVYYCHAYDGRRPPYWGQGTQVTVSS

SEQ ID NO: 176 - protein, 111 aa, synthetic construct
  REGION 1..111: note = Generated by phage peptide display
QVQLQESGGGLVQPGGSLRLSCAASARIFSIYAMVWYRQAPGKQREWVAAITTGGTTNYADSVKGRFTISRDNAKNTVYLQMNSLKPEDTAVYYCNAPGYWGQGTQVTVSS

SEQ ID NO: 177 - protein, 115 aa, synthetic construct
  REGION 1..115: note = Generated by phage peptide display
QVQLQESGGGLVQPGGSLRLSCAASGVIASISVLRWYRQTPGKTRDWVAIITSGGNTRYADSVKGRFTTSRDNARNTVYLQMNSLKPEDTAVYYCNTLVGAKDYWGQGTQVTVSS

SEQ ID NO: 178 - protein, 113 aa, synthetic construct
  REGION 1..113: note = Generated by phage peptide display
QVQLQESGGGLVRPGGSLRLSCEASGTTFRSLVMKWYRQAPGKEREWVAFISSPGDRTRYTEAVKGRFTISRDNAKNALYLQMNGLKPEDTAVYYCNANGIYWGKGTQVTVSS

SEQ ID NO: 179 - protein, 115 aa, synthetic construct
  REGION 1..115: note = Generated by phage peptide display
QVQLQESGGGLVQSGDSLRLSCAASGFTFSNYAMSWVRQAPGKGLEWVSTINSGGGSTSYAYSVKGRFTISRDNAKNTLYLQMNSLKPEDTAVYYCAKTASHIPLSQGTQVTVSS

SEQ ID NO: 180 - protein, 115 aa, synthetic construct
  REGION 1..115: note = Generated by phage peptide display
QVQLQESGGGLVQPGGSLRLSCAASGFTFSNYAMSWVRQAPGKGLEWVSTINIGGGSTSYADSVKGRFTISRDNAKNTLYLQMNSLKPEDTAVYYCAKTASHIPLSQGTQVTVSS

SEQ ID NO: 181 - protein, 117 aa, synthetic construct
  REGION 1..117: note = Generated by phage peptide display
QVQLQESGGGLVQPGGSLRLSCAASGFTFRNYAMSWVRQAPGKGLEWVSTINGGGGITSYADSVKGRFTISRDNAKNTMYLQMNSLKPEDTAVYYCAQTARDSRDSRGQGTQVTVSS

SEQ ID NO: 182 - protein, 117 aa, synthetic construct
  REGION 1..117: note = Generated by phage peptide display
QVQLQESGGGLVQPGGSLRLSCAASGFTFSSYAMSWVRLAPGKGLEWVSTINIAGGITSYADSVKGRFTISRDNAKNTLYLQMNSLKPEDTAVYYCAKTAANWSAQRGQGTQVTVSS

SEQ ID NO: 183 - protein, 117 aa, synthetic construct
  REGION 1..117: note = Generated by phage peptide display
QVQLQESGGGLVQPGGSLRLSCAASGFTFSSYAMSWVRQAPGKGLEWVSTINMGGGTTSYADSVKGRFTISRHNAKNTLYLQMNSLKPEDTAVYYCAKTAGNWSAQRGQGTQVTVSS

SEQ ID NO: 184 - protein, 115 aa, synthetic construct
  REGION 1..115: note = Generated by phage peptide display
QVQLQESGGGLVQPGGSLRLSCAASGFTFSTSAMSWIRQPPGKAREVVATITSAGGSISYVNSVKGRFTISRDNAKNTLYLQMNMLKPEDTAVYYCARLVNLAQTGQGTQVTVSS

SEQ ID NO: 185 - protein, 115 aa, synthetic construct
  REGION 1..115: note = Generated by phage peptide display
QVQLQESGGGLVQPGGSLRLSCAASGFTFSTNAMSWIRQPPGKAREVVATITSAGGSISYVNSVKGRFTISRDNAKNTLYLQMNMLKPEDTAVYYCARLVNLAQTGQGTQVTVSS

SEQ ID NO: 186 - DNA, 393 bp, synthetic construct
  misc_feature 1..393: note = Generated by phage peptide display
  source 1..393: mol_type = other DNA
caggtgcagctgcaggagtctgggggaggcttggtgcaggctgggggctctctgagactctcctgtgcagtttctggaatgatgtacattagggaggctatacgctggtaccgccaggctccagggaagcagcgcgagtgggtcgcctttgtaagtagtactggtaatccacgctatacagactccgtgaagggccgattcaccatctccagagacaacgccaagaacacggtgtatctgcaaatgaacagcctgacacctgaggacacggccgtctattactgtaatacatacttgggctcgagggactactggggccaggggacccaggtcaccgtctccagcggccgctacccgtacgacgttccggactacggttccggccgagcatag

SEQ ID NO: 187 - DNA, 393 bp, synthetic construct
  misc_feature 1..393: note = Generated by phage peptide display
  source 1..393: mol_type = other DNA
caggtgcagctgcaggagtctggaggaggcttggtgcagcctggggggtctctgagactctcctgtgcagtttctggaatgatgtacattaggtacactatgcgctggtaccgccaggctccagggaagcagcgcgagtgggtcgccgttgtaagtagtactggtaatccacactatgcagactccgtgaagggccgattcaccatctccagagacaacgccaagaacacggtgtatctgcaaatgaacagcctgacacctgaggacacggccgtctattactgtaatacatacttgggctcgagggactactggggccaggggacccaggtcaccgtctccagcggccgctacccgtacgacgttccggactacggttccggccgagcatag

SEQ ID NO: 188 - DNA, 381 bp, synthetic construct
  misc_feature 1..381: note = Generated by phage peptide display
  source 1..381: mol_type = other DNA
caggtgcagctgcaggagtctgggggaggcttggtgcggcctggggggtctctgagactctcctgtgcagtctctggaagagccgtcaataggtatcacatgcactggtaccgccaggctccagggaagcagcgcgagtgggtcacatttattagtgttggtggtaccacaaactatgcaggctccgtgaagggccgattcaccgtctcccgagacaacgccaaaaacacgctgtatctgcaaatgaacagcctgaaacctgaggacacggccgtctattactgtaattcagctgaatactggggccaggggacccaggtcaccgtctccagcggccgctacccgtacgacgttccggactacggttccggccgagcatag

SEQ ID NO: 189 - DNA, 396 bp, synthetic construct
  misc_feature 1..396: note = Generated by phage peptide display
  source 1..396: mol_type = other DNA
caggtgcagctgcaggagtctgggggaggctcggtgcagcctggggggtctctgagcctctcctgttcagcctctggaatcatcttcagtgactatgccctgacctggtaccgccaggctccagggaagcagcgcgagtgggttgcacagattacgcgaagtcaaaatataaattatacaggatccgtgaagggccgattcaccgtctccagagacaacgccaagaacacagtgcatctgcaaatgaacagcctgaaacctgaggacacggccgtctactattgtcatgcatatgacggtcgacgcccaccctactggggccaggggacccaggtcaccgtctccagcggccgctacccgtacgacgttccggactacggttccggccgagcatag

SEQ ID NO: 190 - DNA, 396 bp, synthetic construct
  misc_feature 1..396: note = Generated by phage peptide display
  source 1..396: mol_type = other DNA
caggtgcagctgcaggagtctgggggaggctcggtgcagcctggggggtctctgagcctctcctgttcagcctctggaatcatcttcagtgactatgccctgacctggtaccgccaggctccagggaagcagcgcgagtgggttgcacagattacgcgaagccaaaatataaattatacaggatccgtgaagggccgattcaccgtctccagagacaacgccaagaacacagtgcatctgcaaatgaacagcctgaaacctgaggacacggccgtctactattgtcatgcatatgacggtcgacgccgaacctactggggccaggggacccaggtcaccgtctccagcggccgctacccgtacgacgttccggactacggttccggccgagcatag

SEQ ID NO: 191 - DNA, 396 bp, synthetic construct
  misc_feature 1..396: note = Generated by phage peptide display
  source 1..396: mol_type = other DNA
caggtgcagctgcaggagtctgggggaggcttggtgcagcctggggggtctctgagcctctcctgttcagcctctggaatcatcttcagtgactatgccctgacctggtaccgccaggctccagggaagcagcgcgagtgggttgcacagattacgcgaagccaaaatataaattatacaggatccgtgaagggccgattcaccgtctccagagacaacgccaagaacacagtgcatctgcaaatgaacagcctgaaacctgaggacacggccgtctactattgtcatgcatatgacggtcgacgcccaccctactggggccaggggacccaggtcaccgtctccagcggccgctacccgtacgacgttccggactacggttccggccgagcatag

SEQ ID NO: 192 - DNA, 396 bp, synthetic construct
  misc_feature 1..396: note = Generated by phage peptide display
  source 1..396: mol_type = other DNA
caggtgcagctgcaggagtctgggggaggcttggtgcagcctggggggtctctgagcctctcctgttcagcctctggaatcatcttcagtgactatgccctgacctggtaccgccaggctccagggaagcagcgcgagtgggttgcacagattacgcgaaggcaaaatataaattatacaggatccgtgaagggccgattcaccgtctccagagacaacgccaagaacacagtgcatctgcaaatgaacagcctgaaacctgaggacacggccgtctactattgtcatgcatatgacggtcgacgatcaccctactggggccaggggacccaggtcaccgtctccagcggccgctacccgtacgacgttccggactacggttccggccgagcatag

SEQ ID NO: 193 - DNA, 396 bp, synthetic construct
  misc_feature 1..396: note = Generated by phage peptide display
  source 1..396: mol_type = other DNA
caggtgcagctgcaggagtctgggggaggcttggtgcagcctggggggtctctgagactctcctgttcagcctctggaatcatcttcagtgactatgccctgacctggtaccgccaggctccagggaagcagcgcgagtgggttgcacagattacgcgaagtcaaaatataaattatacaggatccgtgaagggccgattcaccgtctccagagacaacgccaagaacacagtgcatctgcaaatgaacagcctgaaacctgaggacgcggccgtctactattgtcatgcatatgacggtcgacgcccaccctactggggccaggggacccaggtcaccgtctccagcggccgctacccgtacgacgttccggactacggttccggccgagcatag

SEQ ID NO: 194 - DNA, 381 bp, synthetic construct
  misc_feature 1..381: note = Generated by phage peptide display
  source 1..381: mol_type = other DNA
caggtgcagctgcaggagtctgggggaggcttggtgcagcctggggggtctctgagactctcctgtgcagcctctgcccgcatcttcagtatctatgccatggtatggtaccgccaggctccagggaagcagcgcgagtgggtcgcagctattactactggtggtaccacaaactatgcagactccgtgaagggccgattcaccatctccagagacaacgccaagaacacggtgtatctgcaaatgaacagcctgaaacctgaggacacggccgtctattactgtaatgctccgggctactggggccaggggacccaggtcaccgtctccagcggccgctacccgtacgacgttccggactacggttccggccgagcatag

SEQ ID NO: 195 - DNA, 393 bp, synthetic construct
  misc_feature 1..393: note = Generated by phage peptide display
  source 1..393: mol_type = other DNA
caggtgcagctgcaggagtctgggggaggcttggtgcagcctggggggtctctgagactctcctgtgcagcctctggagtcatcgccagtatctccgtcctgcgctggtaccgccaaacaccaggaaagacgcgcgactgggtcgcaattattactagtggtggcaacacacgctatgcagactccgtgaagggccgattcaccacctccagagataacgccaggaacacggtgtatctgcaaatgaacagcctgaaacctgaggacacggccgtctattactgtaatacacttgtaggagccaaggactactggggccaggggacccaggtcaccgtctccagcggccgctacccgtacgacgttccggactacggttccggccgagcatag

SEQ ID NO: 196 - DNA, 387 bp, synthetic construct
  misc_feature 1..387: note = Generated by phage peptide display
  source 1..387: mol_type = other DNA
caggtgcagctgcaggagtctgggggaggcttggtgcggcctgggggatctctaagactctcctgtgaagcctctggaaccaccttcagaagcctcgtaatgaaatggtaccgccaggctccagggaaggagcgcgagtgggtcgcatttatttctagtcctggtgatcgcactcgctacacagaagccgtgaagggccgattcaccatctccagagacaatgccaagaacgcgctgtatctgcaaatgaacggcctgaaacctgaggacacggccgtgtattattgtaacgcgaacggaatatactggggcaaagggacccaggtcaccgtctccagcggccgctacccgtacgacgttccggactacggttccggccgagcatag

SEQ ID NO: 197 - DNA, 393 bp, synthetic construct
  misc_feature 1..393: note = Generated by phage peptide display
  source 1..393: mol_type = other DNA
caggtgcagctgcaggagtctgggggaggcttggtgcaatctggggattctctgagactctcctgtgcagcctctggattcaccttcagtaactatgctatgagctgggtccgccaggctccaggaaaggggctcgagtgggtctcaactattaatagtggtggtggtagcacaagctatgcgtactccgtgaagggccgattcaccatctccagagacaacgccaagaacacgctgtatctgcaaatgaacagcctgaaacctgaggacacggccgtgtattactgtgcaaagacggcctctcacatacccttgagccaggggacccaggtcaccgtctccagcggccgctacccgtacgacgttccggactacggttccggccgagcatag

SEQ ID NO: 198 - DNA, 393 bp, synthetic construct
  misc_feature 1..393: note = Generated by phage peptide display
  source 1..393: mol_type = other DNA
caggtgcagctgcaggagtctgggggaggcttggtgcaacctgggggttctctgagactctcctgtgcagcctctggattcaccttcagtaactatgctatgagctgggtccgccaggctccaggaaaggggctcgagtgggtctcaactattaatattggtggtggtagcacaagctatgcagactccgtgaagggccgattcaccatctccagagacaacgccaagaacacgctgtatctgcaaatgaacagcctgaaacctgaggacacggccgtgtattactgtgcaaagacggcctctcacatacccttgagccaggggacccaggtcaccgtctccagcggccgctacccgtacgacgttccggactacggttccggccgagcatag

SEQ ID NO: 199 - DNA, 399 bp, synthetic construct
  misc_feature 1..399: note = Generated by phage peptide display
  source 1..399: mol_type = other DNA
caggtgcagctgcaggagtctgggggaggcttggtgcaacctgggggttctctgagactctcctgtgcagcctctggattcaccttcaggaactatgcaatgagctgggtccgtcaggctccaggaaaggggctcgagtgggtctcaactattaatggtggtggtggtatcacaagctatgcagactccgtgaagggccgattcaccatctccagagacaacgccaagaacacaatgtatctgcaaatgaacagcctgaaacctgaggacacggccgtctattactgtgcccaaaccgcccgcgattcccgcgattcccggggccaggggacccaggtcaccgtctccagcggccgctacccgtacgacgttccggactacggttccggccgagcatag

SEQ ID NO: 200 - DNA, 399 bp, synthetic construct
  misc_feature 1..399: note = Generated by phage peptide display
  source 1..399: mol_type = other DNA
caggtgcagctgcaggagtctgggggaggcttggtgcaacctgggggttctctgagactctcctgtgcagcctctggattcaccttcagtagctatgctatgagctgggtccgcctggctccaggaaaggggctcgagtgggtctcaactattaatatcgctggtggtatcacaagctatgcagactccgtgaagggccgattcaccatctccagagacaacgccaagaacacgctgtatctgcaaatgaacagcctgaaacctgaggacacggccgtgtattactgtgcaaaaacggcggccaactggagcgcccagagaggccaggggacccaggtcaccgtctccagcggccgctacccgtacgacgttccggactacggttccggccgagcatag

SEQ ID NO: 201 - DNA, 399 bp, synthetic construct
  misc_feature 1..399: note = Generated by phage peptide display
  source 1..399: mol_type = other DNA
caggtgcagctgcaggagtctgggggaggcttggtgcaacctgggggttctctgagactctcctgtgcagcctctggattcaccttcagtagctatgctatgagctgggtccgccaggctccaggaaaggggctcgagtgggtctcaactattaatatgggtggtggtaccacaagctatgcagactccgtgaagggccgattcaccatctccagacacaacgccaagaacacgctgtatctgcaaatgaacagcctgaaacctgaggacacggccgtgtattactgtgcaaaaacggcgggcaactggagcgcccagagaggccaggggacccaggtcaccgtctccagcggccgctacccgtacgacgttccggactacggttccggccgagcatag

SEQ ID NO: 202 - DNA, 393 bp, synthetic construct
  misc_feature 1..393: note = Generated by phage peptide display
  source 1..393: mol_type = other DNA
caggtgcagctgcaggagtctgggggaggcttggtgcaacctgggggttctctgagactgtcctgtgcagcctctggattcaccttcagtacaagtgccatgagttggatccgccagcctccagggaaggcgcgcgaggtggtcgcaactattactagtgctggtggtagtataagttatgtaaactccgtgaagggccgattcaccatctccagagacaacgccaagaacacgctgtatctgcaaatgaacatgctgaaacctgaggacacggccgtgtattactgtgcccgactggtcaaccttgcccagaccggccagggaacccaggtcaccgtctccagcggccgctacccgtacgacgttccggactacggttccggccgagcatag

SEQ ID NO: 203 - DNA, 393 bp, synthetic construct
  misc_feature 1..393: note = Generated by phage peptide display
  source 1..393: mol_type = other DNA
caggtgcagctgcaggagtctgggggaggcctggtgcaacctgggggttctctgagactgtcctgtgcagcctctggattcaccttcagtacaaatgccatgagttggatccgccagcctccagggaaggcgcgcgaggtggtcgcaactattactagtgctggtggtagtataagttatgtaaactccgtgaagggccgattcaccatctccagagacaacgccaagaacacgctgtatctgcaaatgaacatgctgaaacctgaggacacggccgtgtattactgtgcccgactggtcaaccttgcccagaccggccaggggacccaggtcaccgtctccagcggccgctacccgtacgacgttccggactacggttccggccgagcatag

SEQ ID NO: 204 - protein, 130 aa, synthetic construct
  REGION 1..130: note = Generated by phage peptide display
QVQLQESGGGMVEPGGSLRLSCAASGFRFSSYAMSWVRQAPGKGLERVSAINSDGDKTSYADSVKGRFTISRDNARNTLYLQMSNLKPEDTAVYYCADRDLYCSGSMCKDVLGGARYDFRGQGTQVTVSS

SEQ ID NO: 205 - protein, 130 aa, synthetic construct
  REGION 1..130: note = Generated by phage peptide display
QVQLQESGGGLVEPGGSLRLSCAASGFRFSSYAMSWVRQAPGKGLERVSAINSDGDKTSYADSVKGRFTISRDNARNTLYLQMSNLKPEDTAVYYCADRDLYCSGSMCKDVLGGARYDFRGQGTQVTVSS

SEQ ID NO: 206 - protein, 130 aa, synthetic construct
  REGION 1..130: note = Generated by phage peptide display
QVQLQESGGGMVEPGGSLRLSCAASGFRFSSYAMSWVRQAPGKGLERVSAINSDGDKTSYADSVKGRFTISRDNARNTLYLQMNNLKPEDTAVYYCADRDLYCSGSMCKDVLGGARYDFRGQGTQVTVSS

SEQ ID NO: 207 - protein, 130 aa, synthetic construct
  REGION 1..130: note = Generated by phage peptide display
QVQLQESGGGLVQSGESLRLSCAASGLRFSSYAMSWVRQAPGKGLERVSAINSDGDKTSYADSVKGRFTISRDNARNTLYLQMSNLKPEDTAVYYCADRDLYCSGSMCKDVLGGARYDFRGQGTQVTVSS

SEQ ID NO: 208 - protein, 123 aa, synthetic construct
  REGION 1..123: note = Generated by phage peptide display
QVQLQESGGGLVQPGGSLKLSCAASGFTFNWYTMAWYRQVPGEERKMVATITGASGDTKYADSVKGRFTISRDNAKNTVTLQMNSLKPGDAAVYYCHAYLTYDSGSVKGVNYWGQGTQVTVSS

SEQ ID NO: 209 - protein, 123 aa, synthetic construct
  REGION 1..123: note = Generated by phage peptide display
QVQLQESGGGLVRPGGSLKLSCAASGFTFNWYTMAWYRQVPGEERKMVATITGASGDTKYADSVKGRFTISRDNAKNTVTLQMNSLKPGDTAVYYCHAYLTYDSGSVKGVNYWGQGTQVTVSS

SEQ ID NO: 210 - protein, 123 aa, synthetic construct
  REGION 1..123: note = Generated by phage peptide display
QVQLQESGGGSVQPGGSLKLSCAASGFTFNWYTMAWYRQVPGEERKMVATITGASGDTKYADSVKGRFTISRDNAKNTVTLQMNSLKPGDTAVYYCHAYLTYDSGSVKGVNYWGQGTQVTVSS

SEQ ID NO: 211 - protein, 123 aa, synthetic construct
  REGION 1..123: note = Generated by phage peptide display
QVQLQESGGGSVQPGGSLKLSCAASGFTFNWYTMAWYRQVPGEERKMVATITGASGDTKYADSVKGRSTISRDNAKNTVTLQMNSLKPGDTAVYYCHACLTYDSGSVKGVNYWGQGTQVTVSS

SEQ ID NO: 212 - protein, 123 aa, synthetic construct
  REGION 1..123: note = Generated by phage peptide display
QVQLQESGGGFVQPGGSLKLSCAASGFTFNWYTMAWYRQVPGEERKMVATITGASGDTKYADSVKGRFTISRDNAKNTVTLQMNSLKPGDTAVYYCHAYLTYDSGSVKGVNYWGQGTQVTVSS

SEQ ID NO: 213 - protein, 123 aa, synthetic construct
  REGION 1..123: note = Generated by phage peptide display
QVQLQESGGGLVQPGGSLKLSCAASGFTFNWYTMAWYRQVPGEERKMVATITGASGDTKYADSVKGRFTISRDNAKNTVTLQMNSLKPGDTAVYYCHAYLTYDSGSAKGVNYWGQGTQVTVSS

SEQ ID NO: 214 - protein, 124 aa, synthetic construct
  REGION 1..124: note = Generated by phage peptide display
QVQLQESGGGLVQPGGSLGLSCAASGSLLNIYAMGWYRQAPGRQRELVATVTSSGTAEYADSVKGRFTISRDNAKNTVYLQMNSLRPEDTGVYYCNAHLRYGDYVRGPPEYNYWGQGTQVTVSS

SEQ ID NO: 215 - protein, 129 aa, synthetic construct
  REGION 1..129: note = Generated by phage peptide display
QVQLQESGGGLVQPGGSLRLSCAASGGTLGYYAIGWFRQAPGKEREAVSCITSSDTSAYYADSAKGRFTISRDNAKNTMYLQMNNLKPEDTAVYYCAAGYYFRDYSDSYYYTGTGMKVWGKGTQVTVSS

SEQ ID NO: 216 - protein, 129 aa, synthetic construct
  REGION 1..129: note = Generated by phage peptide display
QVQLQESGGGLVQPGGSTRLSCAASGFTLDIYAIGWFRQAPGKEHEGVSWIVGNDGRTYYIDSVKGRFTISRDNAKNTVYLEMNSLKPEDTAVYYCAAKFWPRYYSGRPPVGRDGYDYWGQGTQVTVSS

SEQ ID NO: 217 - protein, 111 aa, synthetic construct
  REGION 1..111: note = Generated by phage peptide display
QVQLQESGGGLVQPGGSLILSCTISGASLRDRRVTWSRQGPGKSLEIIAVMAPDYGVHYFGSLEGRVAVRGDVVKNTVYLQVNALKPEDTAIYWCSMGNIRGLGTQVTVSS

SEQ ID NO: 218 - DNA, 438 bp, synthetic construct
  misc_feature 1..438: note = Generated by phage peptide display
  source 1..438: mol_type = other DNA
caggtgcagctgcaggagtctgggggaggcatggtagaacctgggggttctctgagactctcctgtgcagcctctggattccgcttcagtagttatgctatgagttgggtccgccaggctccaggaaaggggctcgagcgggtctcggctattaatagtgatggtgataaaacaagctatgcagactccgtgaagggccgatttaccatctccagagacaacgccaggaacacgctgtatctgcaaatgagcaacctgaaacctgaagacacggccgtgtattactgtgcagaccgagatttgtactgttcaggctctatgtgtaaggacgtcttggggggagcacgctatgactttcggggccaggggacccaggtcaccgtctccagcggccgctacccgtacgacgttccggactacggttccggccgagcatag

SEQ ID NO: 219 - DNA, 438 bp, synthetic construct
  misc_feature 1..438: note = Generated by phage peptide display
  source 1..438: mol_type = other DNA
caggtgcagctgcaggagtctggaggaggcttggtagaacctgggggttctctgagactctcctgtgcagcctctggattccgcttcagtagttatgctatgagttgggtccgccaggctccaggaaaggggctcgagcgggtctcagctattaatagtgatggtgataaaacaagctatgcagactccgtgaagggccgatttaccatctccagagacaacgccaggaacacgctgtatctgcaaatgagcaacctgaaacctgaagacacggccgtgtattactgtgcagaccgagatttgtactgttcaggctctatgtgtaaggacgtcttggggggagcacgctatgactttcggggccaggggacccaggtcaccgtctccagcggccgctacccgtacgacgttccggactacggttccggccgagcatag

SEQ ID NO: 220 - DNA, 438 bp, synthetic construct
  misc_feature 1..438: note = Generated by phage peptide display
  source 1..438: mol_type = other DNA
caggtgcagctgcaggagtctgggggaggcatggtagaacctgggggttctctgagactctcttgtgcagcctctggattccgcttcagtagttatgctatgagttgggtccgccaggctccaggaaaggggctcgagcgggtctcggctattaatagtgatggtgataaaacaagctatgcagactccgtgaagggccgattcaccatctccagagacaacgccaggaacacgctgtatctgcaaatgaacaacctgaaacctgaagacacggccgtgtattactgtgcagaccgagatttgtactgttcgggctctatgtgtaaggacgtcttggggggagcacgctatgactttcggggccaggggacccaggtcaccgtctccagcggccgctacccgtacgacgttccggactacggttccggccgagcatag

SEQ ID NO: 221 - DNA, 438 bp, synthetic construct
  misc_feature 1..438: note = Generated by phage peptide display
  source 1..438: mol_type = other DNA
caggtgcagctgcaggagtctgggggaggcttggtgcagtctggcgagtctctcagactctcctgtgcagcctctggactccgcttcagtagttatgctatgagttgggtccgccaggctccaggaaaggggctcgagcgggtctcggctattaatagtgatggtgataaaacaagctatgcagactccgtgaagggccgatttaccatctccagagacaacgccaggaacacgctgtatctgcaaatgagcaacctgaaacctgaagacacggccgtgtattactgtgcagaccgagatttgtactgttcaggctctatgtgtaaggacgtcttggggggagcacgctatgactttcggggccaggggacccaggtcaccgtctccagcggccgctacccgtacgacgttccggactacggttccggccgagcatag

SEQ ID NO: 222 - DNA, 417 bp, synthetic construct
  misc_feature 1..417: note = Generated by phage peptide display
  source 1..417: mol_type = other DNA
caggtgcagctgcaggagtctggaggaggcctggtgcagcctggggggtctctgaaactctcctgtgcagcctctggattcaccttcaattggtataccatggcctggtatcgccaggttccaggggaggagcgcaaaatggtcgccacaattacaggtgctagtggtgacacaaaatatgcagactccgtgaagggccggttcaccatctccagagacaatgccaagaacacggtgacactgcaaatgaacagccttaaacctggagacgcggccgtctattactgtcatgcctacctaacctacgactcggggtccgtcaaaggagttaactactggggccaggggacccaggtcaccgtctccagcggccgctacccgtacgacgttccggactacggttccggccgagcatag

SEQ ID NO: 223 - DNA, 417 bp, synthetic construct
  misc_feature 1..417: note = Generated by phage peptide display
  source 1..417: mol_type = other DNA
caggtgcagctgcaggagtctgggggaggcttggtgcggcctggggggtctctgaaactctcctgtgcagcctctggattcaccttcaattggtataccatggcctggtatcgccaggttccaggggaggagcgcaaaatggtcgccacaattacaggtgctagtggtgacacaaaatatgcagactccgtgaagggccggttcaccatctccagagacaatgccaagaacacggtgacactgcaaatgaacagccttaaacctggagacacggccgtctattactgtcatgcctacctaacctacgactcggggtccgtcaaaggagttaactactggggccaggggacccaggtcaccgtctccagcggccgctacccgtacgacgttccggactacggttccggccgagcatag

SEQ ID NO: 224 - DNA, 417 bp, synthetic construct
  misc_feature 1..417: note = Generated by phage peptide display
  source 1..417: mol_type = other DNA
caggtgcagctgcaggagtctgggggaggctcggtgcagcctggggggtctctgaaactctcctgtgcagcctctggattcaccttcaattggtataccatggcctggtatcgccaggttccaggggaggagcgcaaaatggttgccacaattacaggtgctagtggtgacacaaaatatgcagactccgtgaagggccggttcaccatctccagagacaatgccaagaacacggtgacactgcaaatgaacagccttaaacctggagacacggccgtctattactgtcatgcctacctaacctacgactcggggtccgtcaaaggagttaactactggggccaggggacccaggtcaccgtctccagcggccgctacccgtacgacgttccggactacggttccggccgagcatag

SEQ ID NO: 225 - DNA, 417 bp, synthetic construct
  misc_feature 1..417: note = Generated by phage peptide display
  source 1..417: mol_type = other DNA
caggtgcagctgcaggagtctgggggaggctcggtgcagcctggggggtctctgaaactctcctgtgcagcctctggattcaccttcaattggtataccatggcctggtatcgccaggttccaggggaggagcgcaaaatggtcgccacaattacaggtgctagtggtgacacaaaatatgcagactccgtgaagggccggtccaccatctccagagacaatgccaagaacacggtgacactgcaaatgaacagccttaaacctggagacacggccgtctattactgtcatgcctgcctaacctacgactcggggtccgtcaaaggagttaactactggggtcaggggacccaggtcaccgtctccagcggccgctacccgtacgacgttccggactacggttccggccgagcatag

SEQ ID NO: 226 - DNA, 417 bp, synthetic construct
  misc_feature 1..417: note = Generated by phage peptide display
  source 1..417: mol_type = other DNA
caggtgcagctgcaggagtctgggggaggcttcgtgcagcctggggggtctctgaaactctcctgtgcagcctctggattcaccttcaattggtataccatggcctggtatcgccaggttccaggggaggagcgcaaaatggtcgccacaattacaggtgctagtggtgacacaaaatatgcagactccgtgaagggccggttcaccatctccagagacaatgccaagaacacggtgacactgcaaatgaacagccttaaacctggagacacggccgtctattactgtcatgcctacctaacctacgactcggggtccgtcaaaggagttaactactggggccaggggacccaggtcaccgtctccagcggccgctacccgtacgacgttccggactacggttccggccgagcatag

SEQ ID NO: 227 - DNA, 417 bp, synthetic construct
  misc_feature 1..417: note = Generated by phage peptide display
  source 1..417: mol_type = other DNA
caggtgcagctgcaggagtctgggggaggcctggtgcagcctggggggtctctgaaactctcctgtgcagcctctggattcaccttcaattggtataccatggcctggtatcgccaggttccaggggaggagcgcaaaatggtcgccacaattacaggtgctagtggtgacacaaaatatgcagactccgtgaagggccggttcaccatctccagagacaatgccaagaacacggtgacactgcaaatgaacagccttaaacctggagacacggccgtctattactgtcatgcctacctaacctacgactcggggtccgccaaaggagttaactactggggccaggggacccaggtcaccgtctccagcggccgctacccgtacgacgttccggactacggttccggccgagcatag

SEQ ID NO: 228 - DNA, 420 bp, synthetic construct
  misc_feature 1..420: note = Generated by phage peptide display
  source 1..420: mol_type = other DNA
caggtgcagctgcaggagtctgggggaggcttggtgcagcctggggggtctctaggactctcctgtgcagcctctggaagcctcttaaatatctatgccatgggctggtaccgccaggctccagggagacagcgcgagttggtcgcaactgtaacgagtagtggaaccgcagaatatgcagactccgtgaagggccgattcaccatctctagagacaacgccaagaacacggtgtatctgcaaatgaacagcctgagacctgaggacacgggcgtctattactgtaatgcacatctcagatatggcgactatgtccgtggccctccggagtataactactggggccaggggacccaggtcaccgtctccagcggccgctacccgtacgacgttccggactacggttccggccgagcatag

SEQ ID NO: 229 - DNA, 435 bp, synthetic construct
  misc_feature 1..435: note = Generated by phage peptide display
  source 1..435: mol_type = other DNA
caggtgcagctgcaggagtctgggggaggcttggtgcagcctggggggtctctgagactctcctgtgcagcctctggaggcactttgggttactatgccataggctggttccgccaggccccagggaaggagcgcgaggcggtctcctgtattactagtagtgacactagcgcatactatgcagactccgcgaagggccgattcaccatctccagagacaacgccaagaacacgatgtatctgcaaatgaacaacctgaaacctgaggacacagccgtttattactgtgcagccggttactattttagagactatagtgacagttactactacacggggacgggtatgaaagtctggggcaaagggacccaggtcaccgtctccagcggccgctacccgtacgacgttccggactacggttccggccgagcatag

SEQ ID NO: 230 - DNA, 435 bp, synthetic construct
  misc_feature 1..435: note = Generated by phage peptide display
  source 1..435: mol_type = other DNA
caggtgcagctgcaggagtctgggggaggcttggtgcagcctggggggtctacgagactctcctgtgcagcctctggattcactttggatatttatgctataggctggttccgccaggccccagggaaggagcatgagggggtctcgtggattgttggtaatgatggtaggacatactacatagactccgtgaagggccgattcaccatctccagagacaacgccaagaacacggtgtatcttgaaatgaacagcctgaaacctgaggatacagccgtttattactgcgcagctaagttctggccccgatattatagtggtaggcctccagtagggagggatggctatgactattggggccaggggacccaggtcaccgtctccagcggccgctacccgtacgacgttccggactacggttccggccgagcatag

SEQ ID NO: 231 - DNA, 381 bp, synthetic construct
  misc_feature 1..381: note = Generated by phage peptide display
  source 1..381: mol_type = other DNA
caggtgcagctgcaggagtctgggggaggcttggtgcagcctggcgggtctctgatactctcctgtacaatctcgggagcctcgctccgagaccgacgcgtcacctggagtcgccaaggtccagggaaatcgcttgagatcatcgcagttatggcgccggattacggggtccattactttggctccctggaggggcgagttgccgtccgaggagacgtcgtcaagaatacagtatatctccaagtaaacgccctgaaacccgaagacacagccatctattggtgcagtatggggaatatccggggcctggggacccaggtcaccgtctccagcggccgctacccgtacgacgttccggactacggttccggccgagcatag